SEQUENCE LISTING (49 sequences)

SEQ ID NO: 1 - protein, 99 aa, Homo sapiens [sequence omitted]

SEQ ID NO: 2 - protein, 63 aa, Homo sapiens
IQRTPKIQVYSRHPAENGKSNFLNCYVSGFHPSDIEVDLLKNGERIEKVEHSDLSFSKDWSFY

SEQ ID NO: 3 - protein, 62 aa, Homo sapiens
IQRTPKIQVYSRHPAENGKSNFLNCYVSGFHPSDIEVDLLKNGERIEKVEHSDLSFSKDWSF

SEQ ID NO: 4 - protein, 61 aa, Homo sapiens
IQRTPKIQVYSRHPAENGKSNFLNCYVSGFHPSDIEVDLLKNGERIEKVEHSDLSFSKDWS

SEQ ID NO: 5 - protein, 31 aa, Homo sapiens
EFTPTEKDEYACRVNHVTLSQPKIVKWDRDM

SEQ ID NO: 6 - protein, 30 aa, Homo sapiens
FTPTEKDEYACRVNHVTLSQPKIVKWDRDM

SEQ ID NO: 7 - protein, 34 aa, Homo sapiens
YYTEFTPTEKDEYACRVNHVTLSQPKIVKWDRDM

SEQ ID NO: 8 - protein, 33 aa, Homo sapiens
YTEFTPTEKDEYACRVNHVTLSQPKIVKWDRDM

SEQ ID NO: 9 - protein, 32 aa, Homo sapiens
TEFTPTEKDEYACRVNHVTLSQPKIVKWDRDM

SEQ ID NO: 10 - protein, 7 aa, Homo sapiens
FYLLYYT

SEQ ID NO: 11 - protein, 3 aa, Homo sapiens
IQR

SEQ ID NO: 12 - protein, 3 aa, Homo sapiens
TPK

SEQ ID NO: 13 - protein, 6 aa, Homo sapiens
IQVYSR

SEQ ID NO: 14 - protein, 7 aa, Homo sapiens
HPAENGK

SEQ ID NO: 15 - protein, 22 aa, Homo sapiens
SNFLNCYVSGFHPSDIEVDLLK

SEQ ID NO: 16 - protein, 21 aa, Homo sapiens
NFLNCYVSGFHPSDIEVDLLK

SEQ ID NO: 17 - protein, 20 aa, Homo sapiens
FLNCYVSGFHPSDIEVDLLK

SEQ ID NO: 18 - protein, 19 aa, Homo sapiens
LNCYVSGFHPSDIEVDLLK

SEQ ID NO: 19 - protein, 17 aa, Homo sapiens
CYVSGFHPSDIEVDLLK

SEQ ID NO: 20 - protein, 16 aa, Homo sapiens
YVSGFHPSDIEVDLLK

SEQ ID NO: 21 - protein, 15 aa, Homo sapiens
VSGFHPSDIEVDLLK

SEQ ID NO: 22 - protein, 14 aa, Homo sapiens
SGFHPSDIEVDLLK

SEQ ID NO: 23 - protein, 13 aa, Homo sapiens
GFHPSDIEVDLLK

SEQ ID NO: 24 - protein, 11 aa, Homo sapiens
HPSDIEVDLLK

SEQ ID NO: 25 - protein, 21 aa, Homo sapiens
SNFLNCYVSGFHPSDIEVDLL

SEQ ID NO: 26 - protein, 20 aa, Homo sapiens
SNFLNCYVSGFHPSDIEVDL

SEQ ID NO: 27 - protein, 19 aa, Homo sapiens
SNFLNCYVSGFHPSDIEVD

SEQ ID NO: 28 - protein, 4 aa, Homo sapiens
NGER

SEQ ID NO: 29 - protein, 3 aa, Homo sapiens
IEK

SEQ ID NO: 30 - protein, 10 aa, Homo sapiens
VEHSDLSFSK

SEQ ID NO: 31 - protein, 11 aa, Homo sapiens
IEKVEHSDLSF

SEQ ID NO: 32 - protein, 12 aa, Homo sapiens
IEKVEHSDLSFS

SEQ ID NO: 33 - protein, 14 aa, Homo sapiens
IEKVEHSDLSFSKD

SEQ ID NO: 34 - protein, 15 aa, Homo sapiens
IEKVEHSDLSFSKDW

SEQ ID NO: 35 - protein, 16 aa, Homo sapiens
IEKVEHSDLSFSKDWS

SEQ ID NO: 36 - protein, 17 aa, Homo sapiens
IEKVEHSDLSFSKDWSF

SEQ ID NO: 37 - protein, 18 aa, Homo sapiens
IEKVEHSDLSFSKDWSFY

SEQ ID NO: 38 - protein, 6 aa, Homo sapiens
DEYACR

SEQ ID NO: 39 - protein, 13 aa, Homo sapiens
EFTPTEKDEYACR

SEQ ID NO: 40 - protein, 12 aa, Homo sapiens
FTPTEKDEYACR

SEQ ID NO: 41 - protein, 11 aa, Homo sapiens
TPTEKDEYACR

SEQ ID NO: 42 - protein, 10 aa, Homo sapiens
PTEKDEYACR

SEQ ID NO: 43 - protein, 9 aa, Homo sapiens
TEKDEYACR

SEQ ID NO: 44 - protein, 8 aa, Homo sapiens
EKDEYACR

SEQ ID NO: 45 - protein, 7 aa, Homo sapiens
KDEYACR

SEQ ID NO: 46 - protein, 10 aa, Homo sapiens
VNHVTLSQPK

SEQ ID NO: 47 - protein, 3 aa, Homo sapiens
IVK

SEQ ID NO: 48 - protein, 5 aa, Homo sapiens
WDRDM

SEQ ID NO: 49 - protein, 3 aa, Homo sapiens
WDR

(19) United States
(12) Patent Application Publication
(10) Pub. No.: US 2009/0093010 A1
(43) Pub. Date: Apr. 9, 2009

(54) Method of Detecting Kidney Dysfunction

(75) Inventors: Peter Nickerson, Winnipeg (CA); John Wilkins, Winnipeg (CA); David Rush, Winnipeg (CA); Stefan Schaub, Magden (CH)

Correspondence Address: PATRICK D. KELLY, 11939 MANCHESTER #403, ST. LOUIS, MO, 63131, US

(73) Assignee: University of Manitoba, Winnipeg, MB (CA)

(21) Appl. No.: 11/575,566

(22) PCT Filed: Sep. 20, 2005
(86) PCT No.: PCT/CA2005/001423

§ 371 Date: Oct. 20, 2008

Related U.S. Application Data
(60) Provisional application No. 60/611,291, filed on Sep. 21, 2004.

(51) Int. Cl. C12Q 1/37; C12Q 1/02

(52) U.S. Cl. 435/23; 435/29

(57) ABSTRACT

Methods and kits for monitoring kidney function, and detecting kidney dysfunction and transplant related disease and rejection are disclosed. The method involves analyzing a sample, such as a urine sample, containing protein from an animal for fragments of β2-microglobulin, wherein the presence of specific β2-microglobulin fragments is indicative of kidney dysfunction and transplant rejection. In another embodiement, urine samples from an animal are tested for protease activity, such as cathepsin D or napsin A, wherein increased protease activity compared to a control sample is indicative of kidney dysfunction.

### FIELD OF THE INVENTION

The present invention relates to methods and kits for monitoring kidney function and detecting kidney dysfunction.

### BACKGROUND OF THE INVENTION

Renal insufficiency is associated with many pathological conditions. Decreased kidney function can be indicative of renal transplant rejection, as well as other organ rejection. Acute tubular necrosis, transient hypertension and preeclampsia during pregnancy, and chronic glomerular diseases can also result in increased proteinuria and enzymuria indicative of decreased kidney function. Furthermore, nephrotoxicity can be secondary to environmental toxic agents such as lead, cadmium, mercury and perchlorethilene as well as pharmaceutical drug toxicity. Therefore, accurate assessment of kidney function has application and significant prognostic value in the clinic.

### Kidney Transplant and Transplant Related Disease

Although short and long-term kidney allograft survival has improved substantially from 1988-1996 (1), this trend did not continue from 1995-2000 (2). Specifically, despite a continuous decrease in reported acute clinical rejection rates within the first year post-transplant in the latter period, allograft recipient survival actually diminished (2). This was attributed to “a higher proportion of acute rejection episodes which have not resolved with full functional recovery in recent years” (2), but it may also be due to undetected—and therefore untreated—rejection episodes (i.e. subclinical rejection) which harm the allograft over time.

Both immunological factors and non-immunological factors (for example calcineurin-inhibitor (CNI)-toxicity, hypertension, and recurrent disease) contribute to the continuous deterioration of allograft function, which is referred to as chronic allograft nephropathy (CAN) (3). Acute allograft rejection is the major immunological risk factor for developing CAN (4,5). However, there remains a consistent rate of late graft loss due to CAN with or without previous acute clinical rejection episodes suggesting the existence of subtle and ‘subclinical’ degrees of graft inflammation that are capable of progressing to CAN. Indeed while non-immunological factors may play a role, a recent analysis found that immunological factors were strong correlates of declining graft function beyond 6 months (6).

Currently about 50% of renal allografts are lost due to patient death with a functioning graft. These patients die mainly from cardiovascular diseases and malignancies (7,8). While this finding may reflect the overall increasing age of renal transplant recipients, it may also be influenced by more potent immunosuppressive regimens used to date, which increase cardiovascular risk factors (e.g. hypertension, hypercholesterinemia) and malignancy development (9). In addition, new emerging opportunistic viral infections such as polyomavirus BK-type nephropathy (10,11) underscore the observation that over-immunosuppression may have increased in current years. With this concern in mind, there has been a recent interest in the implementation of strategies that reduce the net immunosuppression delivered to the patient by avoidance, minimization, withdrawal or substitution drug protocols (12). The problem with such strategies, however, is that there has been to date no way other than a renal allograft biopsy of ascertaining whether the graft is free of rejection, and several attempts at reducing immunosuppression have been followed by acute rejection episodes.

Therefore, the individualization of the immunosuppressive therapy tailored to the needs of every patient at every time point is a major goal. To achieve this, tools to monitor the rejection process in the allograft are mandatory. However, this is complicated by the complex and often redundant biology of allograft rejection.

### Diagnosis of Renal Allograft Rejection

At present, the diagnosis of acute rejection can only be made by renal allograft biopsy, which provides information about the type (humoral vs. cellular) and the severity of rejection (tubulointerstitial vs. vascular) that can be used to select the appropriate anti-rejection therapy. Sometimes it is ‘practical’ in a clinical setting to assume rejection by excluding other possibilities for graft dysfunction and to treat rejection. Nevertheless, most kidney transplant centres perform an allograft biopsy when rejection is a concern and allograft function (measured by serum creatinine) has deteriorated by more than 20-30% from baseline.

However, studies by the Winnipeg Transplant Group have demonstrated that the serum creatinine is an insensitive method for the early detection of renal allograft pathology. Indeed, the histologic criteria for acute rejection are present in 3-45% of protocol biopsies of renal allografts with stable function (‘subclinical rejection’) (42,43,44,45,46). The pathogenic potential of subclinical rejection was demonstrated in a randomized study in which the treatment of early subclinical rejection with corticosteroids improved both early and late outcomes (43). Specifically, there was a decrease in early (months 2-3) as well as late (months 7-12) clinical rejection episodes, a decrease in the chronic tubulointerstitial pathological score at 6 months, and a lower serum creatinine at 24 months in those patients randomized to treatment. Finally, similar to acute pathology, the Winnipeg Transplant Group reported that early chronic allograft pathology, detectable only by a 6-month protocol biopsy (i.e. graft function was stable), is predictive of both a subsequent decline in allograft function and time to graft failure (47,48). These data suggest that early detection and treatment of subclinical inflammation may be required to decrease the incidence of CAN.

With the advent of new immunosuppressive agents it is becoming apparent that a limitation to the ‘gold standard’ (i.e. renal biopsy) is the extent of heterogeneity of inflammation within the allograft resulting in sampling error (49). To overcome this obstacle one could take additional cores, use larger biopsy needles or perform more frequent protocol biopsies. However, clearly this is restricted by patient risk for complications that limits the frequency with which they can be performed, not to mention the associated cost. An alternative is to further increase the baseline immunosuppression for all patients, but this carries the known risks of infection in the short-term, and of drug toxicity and malignancy in the long-term. Therefore, in order to detect and eventually prevent these early pathogenic lesions, it is important to develop non-invasive approaches that sample the entire graft and can be performed repeatedly.

Non-invasive monitoring of the immune response directed at the kidney allograft is constrained to examine cells or proteins from the peripheral blood or urine. Strategies have broadly taken one of two approaches (50). The first takes advantage of donor-recipient MHC disparity, the central target of the alloimmune response, to design donor antigen specific assays. The second strategy is to assess global changes in immune system components of the recipient. As will be discussed below each approach offers distinct advantages and disadvantages. Independent of the strategy however, any clinical assay should be conducted easily with small volumes of blood or urine and be able to be repeated frequently.

### Antigen Specific Assays

These approaches have largely employed donor cells as targets for either recipient T-cells or sera containing antibody targeting donor-MHC. To date the most successful by far has been the ‘cross-match’ assay examining pre-transplant sera for donor specific alloantibodies that target MHC molecules on the surface of donor T- or B-cells (51). In contrast to antibody assays, donor-specific T-cell assays have not proven to be as predictive (50). Tests have included limiting dilution assays (LDA), trans-vivo delayed type hypersensitivity (DTH) assays, enzyme-linked immunospot (ELISPOT) assay, flow cytometry based detection of cytokines, and tetramer staining. Like the antibody ‘cross-match’ assay, the LDA and ELISPOT assays have been successful in detecting pre-transplant donor-specific T-cell memory that predict risk for early acute rejection (52). However, their utility to monitor for acute rejection post-transplant has been rather limited (53). While highly specific for donor antigens, the main disadvantages of these assays are: [I] the need for a repository of donor cells (limits frequency of testing possible), [II] the need for cell expansion (time consuming and labour intensive), [III] reproducibility is poor, [IV] complex interpretation, [V] low sensitivity, [VI] in the case of tetramers requires availability of a diverse panel with a number of potential donor-recipient disparities, and [VII] in the case of trans-vivo DTH the need for a large number of animals (50).

### Antigen Non-Specific Assays

To date antigen non-specific assay development (via immuno-phenotyping for immune cell activation markers, cytokine excretion, or mRNA analysis) has largely been limited to known inflammatory programs that are associated with clinical rejection (50,54,55,56,57,58,59,60). However, it is unclear whether these assays will reliably detect the more subtle (subclinical) forms of acute and/or chronic rejection. The Winnipeg Transplant group attempted to develop non-invasive markers correlating clinical and subclinical rejection with flow based detection of CD69 up-regulation on circulating T-cells (i.e. an early T-cell activation marker that was found in the biopsy infiltrate of acute clinical and subclinical rejection). In this study, CD69 expression tended to correlate with acute allograft inflammation, however, it was also up-regulated when asymptomatic cytomegalovirus (CMV) viremia was present in the blood (61). This study highlighted the difficulty in using antigen non-specific biomarkers; it is difficult to ensure specificity since activation of immune markers in blood can reflect inflammation generated through multiple pathways (i.e. rejection versus infection) and occurring at multiple sites within the patient. In addition, T-cells in the circulation may not necessarily be representative of their abundance within the graft (62). The same problems apply also to studies measuring serum proteins secreted by immune cells (e.g. IL-2, IL-6, IFN-γ). Although statistically significant differences have been found in patients with or without acute rejection, the overlap of the two populations was often substantial (58,59) resulting in either many ‘false positives’ or many ‘false negatives’ for a selected cut-off.

Urine as a specimen for immune monitoring offers some potential advantages compared to serum, because [I] it is in direct contact with the main target of rejection (tubular epithelial cells), [II] it may represent the whole kidney allograft, and [III] it may be less confounded by systemic inflammation. However, urine can be very heterogeneous concerning the amount of cells, the concentration of proteins and the pH. One group used mRNA measurement of granzyme B, perforin and CD103 in urinary lymphocytes to predict acute renal allograft rejection (56,57), others measured cytokines (59) or chemokines (60). Yet again, the major problem in these studies was the insufficient sensitivity and specificity, which limits the clinical usefulness of such assays. The unsatisfactory performances could partially be explained by the rather loose definition of ‘no-rejection’ in these studies, which was mostly based on stable allograft function without further support by allograft histology.

### Urine Biomarkers—β2-Microglobulin as an Indicator of Kidney Dysfunction

Excreted enzymes and low molecular weight proteins have been used as markers of nephron toxicity including transplant rejection. β2-microglobulin is a low molecular weight protein that has been extensively studied for its association with transplant rejection, drug toxicity, and renal proximal tubular function.

β2-microglobulin consists of 99 amino acids with one disulfide bridge and has a molecular weight of 11,731 Da (Swiss-Prot: P61769). It is non-covalently bound to the class I major histocompatibility antigen and found on the cell surface of all nucleated cells. Production of β2-microglobulin is known to be between 150 to 250 mg/day in healthy individuals, whereas an increase is observed in some lymphoproliferative and autoimmune diseases (reviewed in (16,17)). β2-microglobulin is shed from the cell surface and circulates in serum, 98% as a free form (18). Most free β2-microglobulin is filtered by the glomeruli and ≧99.9% reabsorbed by proximal tubular cells (17), where it is thought to be degraded into peptides/amino acids by lysosomes before reuptake into the circulation. Therefore, in healthy individuals with normal proximal tubular function <0.2 mg/L β2-microglobulin is excreted in urine (13,14,15).

Due to these properties, increased intact urinary β2-microglobulin has been considered an ideal biomarker for proximal tubular dysfunction. However, it was soon realized that intact β2-microglobulin values measured by immunoassays decreased significantly over time in urine with a pH<6, suggesting proteolytic activity, which leads to cleaved β2-microglobulin forms that were not detectable by available immunoassays (19,20). This fact largely limited the usefulness of measuring intact urinary β2-microglobulin. Although adding alkali to urine post void can prevent degradation ex vivo, most degradation occurs in vivo, as urine is normally stored in the bladder for at least 2-3 hours prior to voiding. Therefore, the only way to accurately measure intact urinary β2-microglobulin is to give patients alkali (e.g. sodium bicarbonate) systemically to ensure a urine pH≧6 or to analyse only urine samples with pH≧6 (17). By following these steps, the potential of intact urinary β2-microglobulin as a marker for proximal tubular injury has been demonstrated by completely separating patients with lower urinary tract infection from those with pyelonephritis without overlap (15). However, the need for administration of alkali prior to urine analysis and the restriction of using only urine samples with pH≧6 made the measurement of intact urinary β2-microglobulin unattractive for routine clinical use.

Therefore, due to the limitations and problems associated with existing invasive and non-invasive methods of monitoring transplant rejection as well as the need for accurate assessment of kidney function in a plethora of conditions, it would be desirable to identify new non-invasive biomarkers of kidney function and methods of detecting and monitoring these markers in patients.

### SUMMARY OF THE INVENTION

The present inventors have devised a high-throughput method for analyzing the proteome of samples from animals and correlating the protein profile to disease or disorder induced kidney dysfunction. Kidney dysfunction is an indicator of diseases and disorders including but not limited to drug toxicity, heavy metal poisoning, renal tubular damage and other kidney disease, transplant disease including transplant rejection, and systemic diseases such as diabetes, lupus, and rheumatoid arthritis. The inventors have found that the presence of a distinct urinary protein profile correlates with kidney dysfunction.

The inventors have further shown that the distinct protein profile identified from individuals with kidney dysfunction is comprised of cleaved β2-microglobulin protein fragments. These β2-microglobulin protein fragments are useful as diagnostic and prognostic biomarkers of kidney dysfunction. Furthermore, assays for the presence of β2-microglobulin protein fragments may be used to monitor kidney function over time.

The methods of the invention are advantageously non-invasive. The β2-microglobulin protein fragments of the invention are associated with kidney dysfunction and can be detected in urine samples. This allows for frequent measurement, which may further improve clinical outcome by better individualization of therapeutic interventions.

Accordingly, in one embodiment, the present invention provides a method of detecting kidney dysfunction in an animal comprising:

(a) testing a sample from the animal for the presence of β2-microglobulin protein fragments, wherein the presence of one or more β2-microglobulin protein fragments when compared to a control sample indicates that the animal has kidney dysfunction.

In an embodiment of the invention, the β2-microglobulin protein fragments are one or more than one of the fragments selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

In a preferred embodiment of the invention, the sample being tested is urine.

The inventors have also shown that the presence of β2-microglobulin protein fragments indicates that a patient has a transplant related disease, and that the presence of β2-microglobulin protein fragments can be a prognostic indicator of transplant rejection. In particular, patients undergoing subclinical transplant rejection can be identified, permitting immunosuppressive therapies to be tailored to early events in transplant rejection.

Accordingly, in one embodiment, the present invention provides a method of detecting kidney transplant related disease in an animal that has received a transplant comprising:

(a) testing a sample from the animal for the presence of β2-microglobulin protein fragments, wherein the presence of one or more β2-microglobulin protein fragments when compared to a sample from a normal animal indicates that the animal has a kidney transplant related disease.

In a preferred embodiment, a method of the invention is used to detect transplant rejection. In another preferred embodiment, a method of the invention is used to detect subclinical rejection. In yet another preferred embodiment the sample being tested is urine.

Diseases and disorders may induce chronic kidney dysfunction or acute kidney dysfunction. Chronic kidney dysfunction may be interrupted by periods of acute kidney dysfunction. It is necessary to monitor kidney function over time referenced to the individual protein profile over time. Furthermore, repeated testing is desirable to monitor therapeutic efficacy following a particular treatment or course of therapy.

Accordingly, the present invention also provides a method of monitoring kidney function in an animal comprising:

(a) testing a sample from the animal to determine the level of β2-microglobulin protein fragments;

(b) repeating step (a) at a later point in time and comparing the result obtained in step (a) with the result obtained in step (b) wherein a difference in the level of β2-microglobulin protein fragments is indicative of a change in kidney function.

In an embodiment of the invention, the β2-microglobulin protein fragments are one or more than one of the fragments selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

In a preferred embodiment of the invention, the sample being tested is urine.

The present inventors have also shown that protease activity is elevated in the urine of patients with kidney dysfunction.

Accordingly, the present invention further provides a method of detecting kidney dysfunction in an animal comprising:

(a) testing a urine sample from the animal for protease activity, wherein increased protease activity when compared to a control sample indicates that the animal has kidney dysfunction.

In one embodiment, a method of the invention is used to detect transplant rejection. In another embodiment, a method of the invention is used to detect kidney dysfunction induced by a systemic disease selected from the group consisting of diabetes, lupus, or rheumatoid arthritis. In another embodiment a method of the invention is used to detect diabetes induced kidney dysfunction. In yet another embodiment, a method of the invention is used to detect kidney dysfunction induced by drug toxicity. In a preferred embodiment the sample being tested is urine.

The present invention also provides biomarkers that can be used in the detection and prognosis of kidney transplant related disease and which are useful for assessing transplant function and health.

Accordingly, in one embodiment the invention provides a biomarker for detecting kidney dysfunction in an animal comprising at least one β2-microglobulin protein fragment.

Moreover, the present invention provides kits for detecting kidney dysfunction in an animal comprising (i) reagents for conducting a method according to a method of the invention and (ii) instructions for its use.

In a preferred embodiment a kit of the invention is used to detect transplant rejection. In another preferred embodiment, the transplant is a kidney transplant. In yet another preferred embodiment the sample being tested is urine.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 demonstrates the reproducibility of urine protein profiles. One urine sample was applied to 14 spots and analysed. A: Fourteen peaks common to all spectra were selected and compared with regard to their peak intensity by calculating the coefficients of variation. B: Parts of 4 randomly selected spectra from the 14 obtained in A. Manual inspection of the spectra showed the presence of an unlabelled peak (circle in top inset box), although the spectra look the same by “eyeball”.

FIG. 2 demonstrates the impact of storage on urine protein profiles. A: Representative female first-void urine showing the appearance of new peaks (+) in the 2-6 kD range after storage for 3 days at room temperature or at 4° C. B: Representative male mid-stream urine protein profile, which showed only minor changes, whether it was analysed 2 hours after collection or after storage for 3 days at room temperature or at 4° C., respectively.

FIG. 3 demonstrates the impact of freeze-thaw cycles on urine protein profiles. Urine protein profiles obtained before freeze and after 1 to 4 freeze-thaw cycles were unchanged, but an increasing loss of intensity in some peaks was detected (↓). After the 5th freeze-thaw cycle some weak intensity peaks were not detected (−).

FIG. 4 compares first-void and mid-stream urine protein profiles (gel-view). First-void and mid-stream urine protein profiles obtained from three females and three males. In males both urine samples had similar protein profiles, whereas in females there are significant differences. The most prominent difference in female first-void urine are three peaks at 3370.3, 3441.2 and 3484.3 Da (↓), which are consistent with the masses of the α-defensins 2, 1, and 3, respectively (Swiss-Prot P59665+P59666; 3371.9, 3442.5, 3486.5 Da)). The calculated mass accuracy of the SELDI-TOF-MS in this example is <0.07%, which is within the limits given by the manufacturer (<0.1%).

FIG. 5 demonstrates the impact of blood in urine on urine protein profiles. A: Protein profile of urine sample from a healthy male. B: Protein profile after spiking the same sample from A with blood (10 μL blood in 500 μL urine). Four peaks appear which are consistent with the masses of singly- and doubly-charged hemoglobin α- and β-chains (Swiss-Prot P01922: 15126 Da; P02023: 15867 Da). The calculated mass accuracy of the SELDI-TOF-MS in this example is <0.13%, which is slightly above the limits given by the manufacturer (<0.1%). C: Protein profile after centrifugation of the same blood-spiked urine sample from B. Only trace amounts of two of these peaks were detectable (*), however contamination with serum proteins was obvious (e.g. peaks consistent with masses of serum albumin were detected). Albumin has a molecular weight of 66472 Da with its multiply-charged ions at an m/z of 33236 (double-charged), 22157 (triple-charged), 16618 (quadruple-charged), 13294 (quintuple-charged) and 11079 (sextuple-charged).

FIG. 6 demonstrates the impact of dilution on urine protein profiles. Protein profile obtained from A: Urine collected after a 20 h period of no fluid intake; B-E: Serial dilution of urine sample A; F: Urine collected after a 4 L fluid challenge. Starting with a 1:4 dilution, a continuous loss of peaks was observed.

FIG. 7 demonstrates the impact of protein concentration on peak intensity. A: Dilution series of a single protein (ubiquitin, 8565 Da) from 10 pmol/μL to 0.01 pmol/μL (equals 85.6, 8.56, 0.85 and 0.08 ng/μL, respectively). B: Dilution series of ubiquitin from 1 pmol/μL to 0.01 pmol/μL in a mixture of four other proteins with constant concentrations (1.5 pmol/μL dynorphin A, 1 pmol/μL insulin, 0.3 pmol/μL cytochrome C and 0.3 pmol/μL superoxide dismutase). C: Dilution series of ubiquitin from 1 pmol/μL to 0.01 pmol/μL spiked into normal male urine with a protein concentration of 110 mg/L.

FIG. 8 provides an estimation of the detection threshold for urine proteins detected by SELDI-TOF-MS. A, Selected urine proteins with different molecular weights detected by SELDI-TOF-MS (H4-chip) in urine from a healthy person with total urine protein 150 mg/L and urine creatinine 18 mmol/L. The normal concentration range in healthy individuals for hepcidin is about 405 to 4045 pmol/L, for β-defensins is about 2075 to 20755 pmol/L, for β2-microglobulin is about 85250 pmol/L, and for albumin is about 150000 pmol/L. The spectra below show the detection of these proteins by SELDI-TOF-MS. In a 1:4 to 1:16 dilution these proteins are not detectable anymore. Therefore the detection threshold is approximately 10 times below the normal concentration of these proteins (the detection threshold for hepcidin is about 40 pmol/L; for β-defensins is about 200 pmol/L; for β2-microglobulin is about 8500 pmol/L; and for albumin is about 15,000 pmol/L). B, In the previous experiment (FIG. 7C) ubiquitin spiked in urine from the same person was detectable down to 0.1 pmol/mL or 100000 pmol/L, respectively, but not anymore at 0.01 pmol/μL or 10000 pmol/L, respectively. The detection threshold (approximately 11675 pmol/L) lies in the same range as one for β2-microglobulin. C, As an example for chemokine concentration in urine, IP-10 is shown, which was measured by ELISA technology. The normal range for IP-10 in healthy individuals is about 0.9 pmol/L, while values measured during allograft rejection are approximately in the range of 9.2 to 91.9 pmol/L. The anticipated detection threshold for IP-10 by SELDI-TOF-MS is 100-1000 times higher than the measured values during rejection and 10000-100000 times higher than normal values.

FIG. 9 shows representative urine protein profiles. A: Normal control with normal pattern. B: Stable transplant with normal pattern. C: Acute clinical rejection with rejection pattern. D: Glomerulopathy. E: Acute tubular necrosis (ATN). F: Urinary tract infection (UTI). The rejection pattern had prominent peak clusters in three regions corresponding to m/z values of 5270-5550 (Region I; 5 peaks), 7050-7360 (Region II; 3 peaks), and 10530-11100 (Region III; 5 peaks).

FIG. 10 shows a software generated gel-view of urine protein profiles from all groups. Box frames represent the three regions corresponding to m/z values of 5270-5550 (Region I), 7050-7360 (Region II), and 10530-11100 (Region III). * indicates a urine sample with the rejection pattern.

FIG. 11 lists the patients eligible for sequential profiling analysis. Eleven of 22 (50%) in the stable transplant group, and 12 of 18 (66%) in the acute clinical rejection group were eligible for sequential analysis. Exclusion criteria were mainly missing subsequent urine or biopsy samples.

FIG. 12 shows sequential urine protein profiles in representative patients. Urine protein profiles set out at the top left of each of 12A, 12B and 12C are examples of the rejection (Rejn) and the normal (Norm) pattern for comparison. Box frames indicate the three regions corresponding to m/z values of 5270-5550 (Region I), 7050-7360 (Region II), and 10530-11100 (Region III). A, Patient with stable allograft function, normal protocol allograft biopsies, and normal pattern urine protein profiles throughout the post-transplant course. B, Patient with acute clinical rejection (Banff IB) on week 7 post-transplant. After treatment with high dose oral steroids the serum creatinine normalized and remained stable. Subsequent allograft biopsies were normal. The urine protein profile showed the normal pattern 3 week prior to the rejection episode, changed to the rejection pattern at the time of rejection, and returned to the normal pattern consistent with the subsequent allograft biopsies and the allograft function. C, Patient with recurrent acute clinical rejection episodes (Banff IA to IIA). Despite treatment with OKT3, high dose steroids and increased baseline immunosuppression, the patient always exhibited the rejection pattern.

FIG. 13 demonstrates the impact of cell lysis due to freeze-thawing on the detection of the rejection pattern. These spectra show urine from a patient with hematuria and acute clinical allograft rejection. Box frames indicate the regions of interest with the rejection pattern.

FIG. 14 shows the determination of pI of rejection pattern proteins. A: Urine sample with the pattern proteins was dialysed against 50 mM MES pH 6.0 (profile A1) and then incubated with cation-exchange beads. The supernatant was checked for the presence of the pattern protein (profile A2). Subsequently, proteins were eluted from the beads with increasing KCl concentrations. The major fraction containing pattern proteins eluted with 200 mM KCl (profile A3). B: Urine sample with the pattern proteins was dialysed against 50 mM Tris pH 8.0 (profile B1) and then incubated with anion-exchange beads. The supernatant was checked for the presence of the pattern protein (profile B2). Subsequently, proteins were eluted from the beads with increasing NaCl concentrations. The major fraction containing pattern proteins eluted with 200 mM NaCl (profile B3). C: Proteins bind completely to ion exchange sorbents about one pH unit below (cation-exchange sorbents) or above (anion-exchange sorbents) their pI. Therefore, the pI of the pattern proteins can be estimated to be around 7.0.

FIG. 15 illustrates the first step of purification of pattern proteins with cation-exchange beads. [I]: Major components of the urine proteome, their pIs and molecular weights. Uromodulin, albumin, α1-microglobulin, Ig light chains (and IgG), retinol-binding protein and β2-microglobulin are proteins which should not or only partially bind to cation-exchange beads incubated at pH 6.2 due to their pIs. [II]: Purification of pattern proteins on cation-exchange beads at pH 6.2. The box frames indicate the pattern proteins. They are present before incubation to cation-exchange beads (profile A). The supernatant after incubation on cation-exchange beads shows many of the major components of the urine proteome but not the pattern proteins (profile B). These elute with 200 mM KCl, notably without a significant contamination with albumin, Ig light chain, retinolbinding protein and β2-microglobulin (profile C). As expected from their contribution to the total protein content of urine, the purification on the cation-exchange beads at pH 6.2 resulted in a significant decrease of the total protein concentration in the elution fraction from 1.92 g/L (before incubation on beads, A) to 0.2 g/L (C) (measured with the BCA protein assay, Pierce, Rockford, Ill., USA). Therefore, the use of the cation-exchange beads as a first step of purification can not only concentrate the pattern proteins but can also separate them from many of the major protein components in urine (e.g. albumin, Ig light chain, retinol-binding protein, β2-microglobulin).

FIG. 16 illustrates the final purification of the protein peak cluster by RP-HPLC. 1, Chromatogram with the peak fraction containing the protein peak cluster (Fraction A, shaded area under curve) and intact β2-microglobulin (Fraction B, shaded area under curve). The ascending graph line indicates the gradient of acetonitrile [%]. 2, SELDI-TOF-MS spectra of fraction A and B. Spectrum A shows the three previously described peak clusters (I, II, and III) and an additional prominent cluster (IV) consisting of two major peaks at 3608.7 Da and 3738.4 Da. Spectrum B shows three peaks, which represent single-(11731.0 Da), double-(5864.9 Da), and triple-charged (3909.3 Da) intact β2-microglobulin.

FIG. 17 shows the peptides of the β2-microglobulin sequence identified by μLC-MALDI-MS (/MS). β2-microglobulin consists of 99 amino acids (SEQ ID NO:1). All peptides covering the whole β2-microglobulin sequence were found and confirmed by μLC-MALDI-MS (/MS), except a missing piece from L64-T68 (“Missing piece”). In addition, 26 non-tryptic cleaved peptides were found. Trypsin cleavage sites (R and K) are indicated with arrows (→), non-tryptic cleavage sites with filled circles ().

FIG. 18 shows an explanation of SELDI-TOF-MS-detected protein peak clusters with cleaved forms of β2-microglobulin. 1, Intact β2-microglobulin consists of 99 amino acids with one disulfide bond (C25-C80). Initial non-tryptic cleavages remove the peptide piece L64-T68. 2, These cleavages create two chains connected by the disulfide bond. Thereafter, additional cleavages occur at S61 and F62 (on the long chain), as well as at E69 (on the short chain). This results in three different forms of the long chain (I1-Y63, I1-F62, I1-S61), two different forms of the short chain (E69-M99, F70-M99), and consequently six different forms with the intact disulfide bond. I denotes a major cleavage site. 3, SELDI-TOF-MS spectrum of a urine sample with the characteristic protein peak clusters. During analysis by SELDI-TOF-MS disulfide bonds can break resulting in the detection of the different forms of the long chain (II) and the short chain (IV). In addition, forms with intact disulfide bonds (III=single charged ions; I=double-charged ions) are detectable. However, the resolution of the SELDI-TOF-MS did not allow the separation of all six forms with intact disulfide bonds into individual peaks. Predicted peaks with calculated average masses of 10930.23 Da and 10783.05 Da were not resolvable (n.r.). The expected mass accuracy of SELDI-TOF-MS as given by the manufacturer is <0.1% (<1 Da per 1000 Da). The difference between the calculated average masses and the observed masses by SELDI-TOF-MS are given as mass [Da].

FIG. 19 illustrates the detection limits of SELDI-TOF-MS. Ovals represent normal (hepcidin, α-defensins, β2-microglobulin, retinol-binding-protein (RBP) and albumin), and pathological concentrations (IL-2 to IL-6, IFN-γ, IP-10, Interferon gamma inducible T-cell chemotactant (I-TAC), Monokine induced by interferon gamma (Mig), Prostate specific antigen (PSA) and α-fetoprotein) of known urine and serum proteins measured by ELISA or equal quantitative tests. Hepcidin, α-defensins, β2-microglobulin and albumin are detectable by SELDI-TOF-MS in urine from healthy individuals. From dilution experiments, it is known that in a 1:4-1:16 urine dilution these proteins are not detectable anymore. In addition, ubiquitin (spiked in normal urine) was detectable until 100000 pmol/L but not at 10000 pmol/L. Therefore, the SELDI-TOF-MS-detection threshold of these proteins in urine can be approximated (shaded upper area). Single proteins (α-defensins, ubiquitin and albumin) are detectable by SELDI-TOF-MS at roughly 100 times lower concentrations than in urine. However, even this threshold is above the level of cytokines, chemokines and currently used tumour maker (i.e. PSA and α-fetoprotein). This graphic also shows the high dynamic range of urine and serum proteins, which spans over 7-10 log units.

FIG. 20 illustrates the pathways which may be involved in cleavage of β2-microglobulin, β2-microglobulin is freely filtered through the glomerular barrier and reabsorbed to a large (but unknown) extent by tubular epithelial cells. It can by transferred directly back into the bloodstream, but it may also by degraded in lysosomes. Resulting fragments may be brought back into the blood stream, but regurgitation of fragments into the urine is also possible. Tubular epithelial stress/injury due to rejection may enhance regurgitation of fragmented proteins into urine and decrease their transport into blood stream (vertical double-line (II) at right side of illustration). However, β2-microglobulin may also be cleaved intraluminally by proteinases released by tubular epithelial cells, CTL and macrophages.

FIG. 21 illustrates the determination of the protease family responsible for β2-microglobulin cleavages. A urine sample from a healthy individual was mixed with different protease inhibitors (PI) and different buffers before adding intact β2-microglobulin (rows A to E). These samples were then analysed by SELDI-TOF-MS immediately (column 1) and after 6 hours of incubation at 37° C. (column 2). The three characteristic protein peak clusters resulting from β2-microglobulin cleavages are marked as shaded areas with roman numbers II, III and IV. Only pepstatin could inhibit β2-microglobulin cleavage at pH 5 (row B), however, at urine pH 6 no cleavage could be detected even without addition of protease inhibitors (row E). The same results were obtained with a urine sample from a patient with an acute clinical rejection episode (data not shown). β2-m⁺⁽⁺⁾=single and double-charged intact β2-microglobulin.

FIG. 22 shows the estimated protease amount in different urine samples. Urine from a healthy individual (column 1) and a patient with an acute clinical rejection episode (column 2) was analysed by SELDI-TOF-MS after collection (row A). The three characteristic protein peak clusters resulting from β2-microglobulin cleavages are marked as shaded areas with roman numbers II, III and IV. These samples were then adjusted to pH 5 and incubated for 16 hours at 37° C. which leads to almost complete degradation of existing intact and cleaved β2-microglobulin forms (row B). Subsequently, equal amounts of purified intact β2-microglobulin (final concentration 10 mg/L) were added to both samples (row C). Samples were again incubated at 37° C. and analysed after one (row D) and six hours (row E). The acute clinical rejection urine sample showed the characteristic protein peak clusters of cleaved β2-microglobulin forms after one hour (2D), whereas after six hours degradation was almost complete (2E). In contrast, the healthy individual urine sample showed the characteristic protein peak clusters of cleaved β2-microglobulin forms after six hours (1E), whereas complete degradation was achieved after 24 hours (data not shown). β2-m⁺⁽⁺⁾=single and double-charged intact β2-microglobulin.

FIG. 23 shows the estimated detection threshold of SELDI-TOF-MS for cleaved β2-microglobulin. A urine sample from a healthy individual was depleted of intact and cleaved β2-microglobulin by incubating for 16 hours at 37° C. Then different amounts of intact β2-microglobulin were added (row A-E) and the samples were analysed by SELDI-TOF-MS immediately (column 1) and after 6 hours at 37° C. (column 2). The three characteristic protein peak clusters resulting from β2-microglobulin cleavages are marked as shaded areas with roman numbers II, III and IV. They were detectable down to an added intact β2-microglobulin concentration of 0.5 mg/L, but not at 0.1 mg/L. β2-m⁺⁽⁺⁾=single and double-charged intact β2-microglobulin.

FIG. 24 illustrates the pathogenesis of cleaved urinary β2-microglobulin. Tubular cell stress/injury can (i) decrease reabsorption of filtered intact β2-microglobulin, (ii) increase the amount of proteases in urine (e.g. through tubular cell death (31) or regurgitation of lysosomal contents (32,33)), and (iii) decrease urine pH. Under such conditions substantial amounts of cleaved β2-microglobulin are generated, which may account for the major part of total urinary β2-microglobulin (=intact and cleaved β2-microglobulin).

### DETAILED DESCRIPTION OF THE INVENTION

Assessment of kidney function is a prognostic indicator of disease progression and can be used to determine adequacy of treatment. As described above, the available methods of assessing kidney function are inadequate for detecting early disease progression.

Renal insufficiency is associated with many pathological conditions. Decreased kidney function can be indicative of renal transplant rejection, as well as other organ rejection. Acute tubular necrosis, transient hypertension and preeclampsia during pregnancy, and chronic glomerular diseases can also result in increased proteinuria and enzymuria indicative of decreased kidney function (119). Diabetes and cancer can also impact kidney function. Furthermore, nephrotoxicity can be secondary to environmental toxic agents such as lead, cadmium, mercury and perchlorethilene as well as pharmaceutical drug toxicity (119). Hence accurate assessment of kidney function has application and significant prognostic value in the clinic.

The present inventors have provided non-invasive methods for the monitoring of kidney function and detection of kidney dysfunction and kidney transplant related disease, based on the presence of β2-microglobulin protein fragments.

Accordingly, in one embodiment, the present invention provides a method of detecting kidney dysfunction in an animal comprising:

(a) testing a sample from the animal for the presence of β2-microglobulin protein fragments, wherein the presence of one or more β2-microglobulin protein fragments when compared to a control sample indicates that the animal has kidney dysfunction.

The term “sample from the animal” as used herein means any sample including, but not limited to, biological fluids, tissue extracts, freshly harvested cells, and lysates of cells which have been incubated in cell cultures. In a preferred embodiment, the sample is urine.

As used herein the phrase “β2-microglobulin protein fragments” or “fragments of the β2-microglobulin protein” means a fragment or portion of the full length β2-microglobulin protein and includes polymorphic versions of amino acid sequences of all of the known β2-microglobulin molecules and precursor molecules, including those deposited in GenBank under accession number CAA23830 or those referred to in Suggs et al. Proc. Natl. Acad. Sci. U.S.A. 78 (11), 6613-6617 (1981), as well as modified versions including those referred to in Momoi et al., Clin Chim Acta. 1995 May 15; 236(2):135-44, and any variants, analogs, derivatives or portions thereof that are useful in detecting transplant related disease.

The term “animal” as used herein includes all members of the animal kingdom, including humans. Preferably, the animal is a human.

In a preferred embodiment, β2-microglobulin protein fragments are selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9) (letters indicate single letter amino acid code; numbers indicate position of amino acids in full-length β2-microglobulin protein sequence). β2-microglobulin protein giving rise to the β2-microglobulin protein fragments may be cleaved at one or more of the following sites: tyrosine-63 (Y-63), leucine-65 (L65), phenylalanine-62 (F62), and serine-61 (S61). The major distinct protein fragments resulting from these cleavages may have the approximate molecular weights of 7358 Da, 7195 Da or 7048 Da. Additionally the β2-microglobulin long chain may be cleaved at one or more of the following sites: phenylalanine-22 (F-22), asparagine-24 (N24) and cysteine-25 (C-25). Fragments resulting from these cleavages may or may not be detectable. The β2-microglobulin protein may or may not also be cleaved in its short chain at lysine-75 (K75), glutamic acid-74 (E74), threonine-73 (T73), proline-72 (P-72), threonine-71 (T71), phenylalanine-70 (F70) and/or glutamic acid-69 (E69). Two fragments resulting from these cleavages may have the approximate molecular weight of 3737 Da and 3608 Da.

The term “control sample” includes any sample that can be used to establish a base or normal level, and may include samples taken from healthy animals or samples mimicking physiological fluid.

As used herein “kidney dysfunction” means abnormal tubular function resulting in the loss of proteins into the urine that are normally absent from the urine.

As used herein “non-invasive” refers to a method whereby the sample to be tested can be obtained without biopsy. Preferably “non-invasive” refers to a method whereby the sample to be tested can be obtained without puncturing the skin of the animal.

As used herein “protein profile” means the group of protein fragments obtained from a sample and is used interchangeably with “distinct protein profile” or “protein profile pattern” or “protein pattern”. The protein profile can indicate whether the animal has a kidney dysfunction related disease or disorder such as a transplant rejection.

Diseases and disorders may induce chronic kidney dysfunction or acute kidney dysfunction. Chronic kidney dysfunction may be interrupted by periods of acute kidney dysfunction. It is necessary to monitor kidney function over time referenced to the individual protein profile over time. Furthermore, repeated testing is desirable to monitor therapeutic efficacy following a particular treatment or course of therapy. Therefore, the methods of the invention are also used to monitor the adequacy of therapeutic interventions.

Accordingly, the present invention also provides a method of monitoring kidney function in an animal comprising:

(a) testing a sample from the animal to determine the level of β2-microglobulin protein fragments;

(b) repeating step (a) at a later point in time and comparing the result obtained in step (a) with the result obtained in step (b) wherein a difference in the level of β2-microglobulin protein fragments is indicative of a change in kidney function.

### Transplant Rejection

There are four main categories responsible for allograft injury: (i) rejection episodes, (ii) drug-toxicity (i.e. calcineurin-inhibitors (CI)), (iii) specific diseases (e.g. polyomavirus type BK-nephropathy, recurrent disease in the allograft), and (iv) disease accelerating factors (e.g. hypertension, diabetes) (41). Acute clinical rejection is the major risk factor for allograft failure (4), but even rejection episodes without allograft dysfunction as measured by serum creatinine (i.e. subclinical rejection detected by protocol biopsies) can lead to chronic allograft nephropathy (116,117). Moreover, CI-nephrotoxicity was reported in >50% of protocol biopsies performed after the second year post-transplant (42). However, protocol biopsies have not gained widespread acceptance due to their associated costs, inconvenience and morbidity. Non-invasive biomarkers in serum or urine, which can be measured frequently, may guide the clinical decision to perform an allograft biopsy. Indeed, sensitive, non-invasive biomarkers of tissue injury may allow the clinician to determine its cause (i.e. by allograft biopsy) before irreversible damage has occurred. Furthermore, the response to therapeutic interventions can be followed by frequent measurement of such biomarkers (118).

Post-transplant immune monitoring of renal transplant recipients is currently based on the integrated information gathered from the allograft function (i.e. serum creatinine), the risk profile of a patient (e.g. number of MHC-mismatches, presensitization), the clinical course (e.g. prior rejections) and ultimately the allograft biopsy results. While these tools have proved to be invaluable for adjusting the immunosuppressive therapy, they still have major shortcomings as described above.

Immune monitoring with non-invasive markers, which allows for frequent measurement, may further improve the clinical outcome of the allograft recipient by better individualization of immunosuppressive therapy. Specifically, this includes reduction of immunosuppressive therapy for patients inferred to be free of rejection by the non-invasive test, as well as increasing immunosuppressive therapy before tissue damage occurs and the rejection process becomes obvious (i.e. worsening allograft function). Non-invasive, antigen-specific tests are mostly labour intensive, expensive and required donor cells (with the exception of tetramer-staining), and do not lend themselves to high-throughput analysis in busy clinical settings. Non-antigen specific tests are cheaper and have high-throughput capabilities, but they often lack sensitivity and specificity for allograft rejection. As urine is [I] in direct contact with the main target of rejection (tubular epithelial cells), [II] may represent the whole kidney allograft, and [III] may also be less confounded by systemic inflammatory processes, non-invasive biomarkers in urine may have a higher sensitivity and specificity than serum biomarkers. Finally, proteins, as the effector molecules, may be more informative and specific for the rejection process than gene transcription products (i.e. mRNA).

Accordingly, in one embodiment, the present invention provides a method of detecting kidney transplant related disease in an animal that has received a transplant comprising:

(a) testing a sample from the animal for the presence of β2-microglobulin protein fragments, wherein the presence of one or more β2-microglobulin protein fragments when compared to a sample from a normal animal indicates that the animal has a kidney transplant related disease.

In a preferred embodiment a method of the invention is used to detect transplant rejection.

As used herein “transplant” means a tissue or organ transplanted from a donor of the same or of a different species and includes allografts and xenografts. Furthermore “transplant” includes solid organ transplants and kidney transplants.

As used herein “transplant related disease” comprises illnesses and conditions affecting the transplant such as transplant rejection, acute allograft rejection, subclinical rejection episodes, interstitial fibrosis, fibrous intimal thickening of arteries, and calcineurin-inhibitor toxicity. When referring to a kidney transplant, “transplant related disease” further comprises tubular stress and injury, tubular atrophy, glomerulosclerosis, polyomavirus type BK-nephropathy (BK-NP), chronic allograft nephropathy (CAN), and pyelonephritis (PN).

As used herein “transplant rejection” means the presence of an immunological inflammatory response in the transplant. With respect to kidney transplants, it means the presence of an immunological inflammatory response in the kidney transplant that is targeting the tubulointerstitial compartment of the kidney. When the transplant related disease is transplant rejection, the distinct protein profile identified following analysis of urine samples is sometimes referred to as a “rejection pattern”.

Currently about 50% of kidney transplants are lost due to patient death with a functioning graft. The potent immunosuppressive regimens used to date increase cardiovascular risk factors such as hypertension and hypercholeserinemia and increase malignancy development (9), which may contribute to transplant patient death rates. Over-immunosuppression may also increase the risk for developing opportunistic infections, which may further complicate transplant management. The invention provides a non-invasive method of detecting a transplant related disease that can be performed repeatedly and analyzed quickly. One of the advantages of the current invention is that the non-invasive nature of the methods permits repeated testing and better individualization of immunosuppressive therapies.

The sample tested may be serum, blood, urine or tissue. Urine as a specimen for immune monitoring in renal transplants offers some potential advantages compared to serum. It is in direct contact with the main target of rejection and may represent the whole kidney transplant. Furthermore it may be less confounded by systemic inflammation. In a preferred embodiment of the invention, the animal sample tested is urine. In a further preferred embodiment, the urine sample is a mid-stream urine sample.

### Monitoring Kidney Dysfunction as an Indicator of Transplant Health

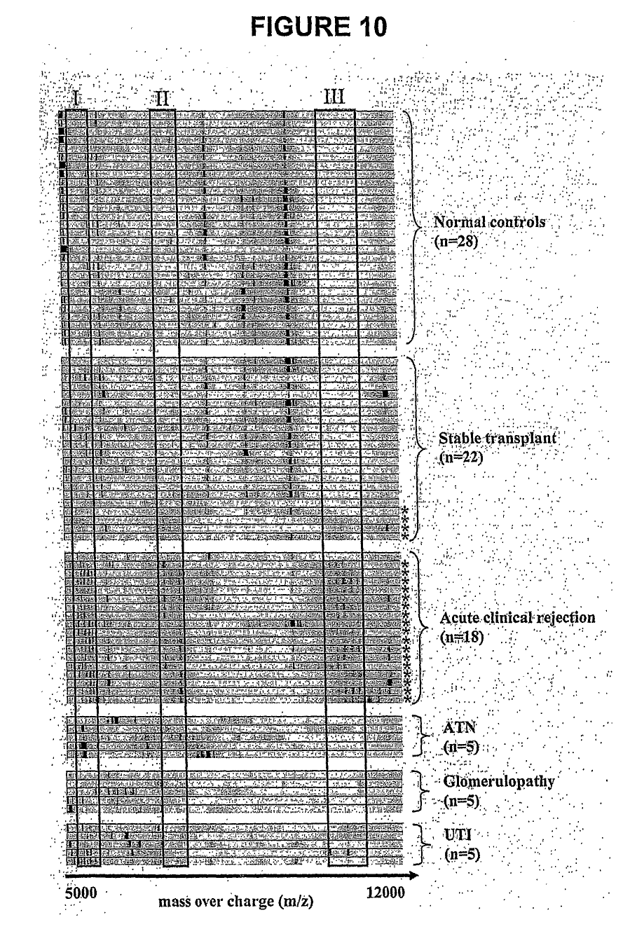

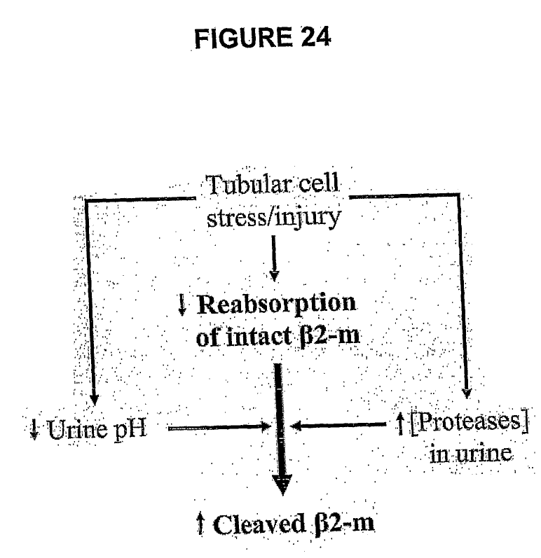

FIG. 10 reveals that seventeen of 18 patients (94%) in the acute clinical allograft rejection group (see Example 5) had cleaved β2-microglobulin detectable by SELDI-TOF-MS, but only 4 of 22 patients (18%) without clinical and histological evidence for rejection and 0 of 28 normal controls. Cleaved urinary β2-microglobulin can be regarded as a marker for tubular cell stress/injury, because all patients in the acute clinical allograft rejection group had at least mild tubulitis (i.e. Banff acute Score ≧i2t2). Therefore, tubular cell stress/injury during allograft rejection can lead to (i) decreased reabsorption of intact β2-microglobulin, (ii) increased amounts of proteases in urine, and (iii) lower urine pH (FIG. 24). Therefore, cleaved urinary β2-microglobulin represents several pathophysiological processes occurring during tubular cell stress/injury related to tubulointerstitial allograft rejection. However, cleaved urinary β2-microglobulin is not likely specific for tubulointerstitial allograft rejection, but may be a sensitive marker for tubular cell stress/injury (e.g. CI-nephrotoxicity, polyomavirus type BK-nephropathy).

In a further aspect of the present invention, a non-invasive method for the detection and monitoring of transplant health and for the early detection and monitoring of transplant rejection is provided.

Accordingly, in one embodiment the invention provides a method of monitoring transplant health in an animal comprising:

(a) testing a sample from the animal to determine the level of β2-microglobulin protein fragments;

(b) repeating step (a) at a later point in time and comparing the result obtained in step (a) with the result obtained in step (b) wherein a difference in the level of β2-microglobulin protein fragments is indicative of a change in transplant health.

As used herein “transplant health” means an assessment of organ function that is compared to a clinically defined normal organ function (i.e. based on creatinine levels) or “normal” transplant function.

The inventors have shown that the presence of a protein profile indicative of transplant related disease, in particular the presence of β2-microglobulin protein fragments, precedes other measures of clinical rejection (i.e. defined change in serum creatinine levels). The invention permits, in one embodiment, the identification of individuals undergoing subclinical rejection. This allows for greater individualization of immunosuppressive therapies. Studies have demonstrated the pathogenic potential of subclinical rejection and early treatment can improve both early and late outcomes (43). Monitoring transplant health is advantageous since it allows for the reduction of immunosuppressive therapy for patients inferred to be free of rejection. It further permits for immunosuppressive therapies to be augmented or altered before tissue damage occurs and the rejection process becomes obvious (i.e. worsening allograft function). One of the advantages of the current invention is that the non-invasive nature of the methods permits repeated testing and better individualization of immunosuppressive therapies.

As used herein “subclinical rejection” means stable transplant function but wherein the transplant exhibits some histologic criteria of acute rejection.

In a preferred embodiment a method of the invention is used to detect or monitor sub-clinical transplant rejection. In a further preferred embodiment, the transplant is a kidney transplant. In another preferred embodiment the sample being tested is urine.

### Methods of Detecting a Distinct Protein Pattern

A protein profile can be assessed by one of several methods including, but not limited to, gel electrophoresis including 2D gel electrophoresis; chromatography including liquid chromatography; protein microarray; isotope coded affinity tags; hydrolytic labeling; and mass spectrometry including SELDI-TOF-MS. In a preferred embodiment the protein profile is detected using a SELDI-TOF-MS platform.

SELDI-TOF-MS provides many advantages for the protein profiling of urine samples. A small volume of sample (i.e. 5-10 μL) is needed for each analysis and many samples can be analyzed quickly. This permits high-throughput profiling of many samples. Furthermore, washing steps are easily incorporated and this has the advantage of removing most of the salts, which interfere with mass spectrometric analysis.

Other groups have used SELDI-TOF-MS to compare the protein profiles between different clinical outcomes, but required bioinformatic analysis to assign protein peaks to a specific outcome (98,99). In another study, Clarke et al. (98) reported differences in the urine profiles between rejection and stable transplants; however, Clarke et al.'s requirement of bioinformatics to do so may relate to the fact that their definition of ‘stable’ transplants was less stringent than that of the present inventors (i.e. based on serum creatinine alone). Interestingly, the protein peaks reported in the Clarke et al. paper as specific to rejection, are different from those found by the present inventors. This may be related to the different protein chip surfaces and experimental conditions that were utilized; but also, to the fact that Clarke et al. (98) failed to include any control populations (e.g. ATN, recurrent or de novo glomerulopathies, UTI, CMV) in the analysis, the importance of which is discussed below. In another study, Petricoin et al. (99) have used SELDI-TOF-MS to compare the protein profiles between different clinico-pathological diagnoses in cases of ovarian cancer, but also required bioinformatic analysis to assign peaks to specific outcomes. In the Petricoin et al. study the analysis involved serum samples which is clearly a more complex biological fluid than urine. Indeed, the urine-based proteomics has the advantage of excluding most of the serum proteins from the urine due to the size/charge selectivity of the glomerular basement membrane.

In a preferred embodiment, the protein profile detected using SELDI-TOF-MS is comprised of 1-3 Regions or clusters of one or more distinct protein fragments. In one embodiment Region 1 preferably consists of 5 distinct fragments. In another embodiment Region 2 preferably consists of 3 distinct fragments. In a further embodiment the distinct fragments are clustered in three regions, wherein Region 1 comprises 5 fragments; Region 2 consists of 3 fragments; and Region 3 consists of 5 fragments.

### Quantitative Assay

The present invention also provides quantitative assays for detecting protein fragments. These quantitative assays permit the detection of changes in concentration of intact protein, of protein fragments, and of intact protein and fragments, and may be immunological in nature. Immunological assays can be based on: (i) the detection of neoepitopes arising as a result of cleavage of intact protein or protein fragments; (ii) the determining of the ratio of binding of antibodies directed at different epitopes present on the whole molecule or fragments thereof, wherein the loss of epitopes (i.e. cleavage of intact protein or protein fragments) would cause a shift; or (iii) the appearance of fragments which could be captured and displayed using a range of different physical methods, for example polyacrylamide gel electrophoresis or mass spectrometry.

In a preferred embodiment a method of the invention is used to detect transplant rejection. In a further preferred embodiment, the transplant is a kidney transplant. In yet another preferred embodiment the sample being tested is urine.

### Biomarkers

The present invention also provides biomarkers that can be used in the detection and prognosis of kidney transplant related disease and which are useful for assessing transplant function and health.

Accordingly, in one embodiment the invention provides a biomarker for detecting kidney dysfunction in an animal comprising at least one β2-microglobulin protein fragment.

As used herein “biomarker” means at least one protein fragment that can be used for one or more of the following: to detect that an animal has a disease; to predict that an animal will develop a disease; to monitor the progression of a disease; or to monitor the effect of a treatment.

A biomarker may have various uses. An early intervention (or diagnostic) biomarker is used for early detection of disease to facilitate intervention. A prognostic biomarker is used to identify patients who may benefit from an intervention (63). Ideally, a biomarker has both, diagnostic and prognostic properties.

A diagnostic biomarker is described by its sensitivity, specificity and its receiver operating characteristics (ROC) curve. ROC-analysis allows finding the best cut-off value to assign the test result to be ‘positive’ or ‘negative’. For clinical decision-making, it is more important to know the positive (PPV; ‘true positives’) and negative predictive value (NPV; ‘true negatives’) than its sensitivity and specificity. This calculation then allows determination of how many ‘false positive’ and ‘false negative’ results the test produces. These numbers should be as low as possible, because they represent the patients that are wrongly assigned to have either a ‘positive’ or a ‘negative’ test. Besides the given and constant factors that affect sensitivity and the specificity of a diagnostic test, the prevalence of the target disease in the screened population largely influences the PPV, the NPV, the number of ‘false positives’ and the number of ‘false negatives’. Therefore, these values should always be calculated based on the ‘true prevalence’ of the disease in the screened population rather than from a selected population, which may over- or underestimate the ‘true prevalence’ and consequently lead to wrongly calculated PPV and NPV (64).

A prognostic biomarker should preferably ‘predict’ the outcome of a particular condition. Prediction requires the further criterion of showing that changes in the value have consequential changes in the outcome. Many prognostic biomarkers used to date only ‘correlate’ with an outcome (e.g. C-reactive protein and risk of acute myocardial infarction), fewer ‘predict’ (e.g. smoking and risk of lung cancer or acute myocardial infarction).

Serum β2-microglobulin protein levels have been found to increase in patients undergoing renal transplant rejection (Backman L et al. Transplantation 42: 368, 1986) and heart transplant (Erez E et al. J. Heart Lung Transplant 17: 538, 1998) and increased expression of β2-microglobulin has been observed in the bile ducts, hepatocytes and endothelial cells of patients undergoing liver transplant rejection (Hubscher S G et al J. Clin Pathol. 41: 1049). Urine β2-microglobulin levels have also been examined for a potential association with transplant rejection but the results have been conflicting. Prischl and colleagues reported that out of 100 episodes of clinical rejection, 50 had only a moderate increase in urine β2-microglobulin levels (Prischl F et al. Nephron 1989; 51(3):330-7) and others found no increase in urine β2-microglobulin during episodes of renal rejection (Steinhoff J et al. Clin Nephrol. 1991 June; 35(6):255-62). The inventors have found that fragments of β2-microglobulin detectable in urine can serve as biomarkers for transplant rejection.

In one embodiment a biomarker of the invention comprises at least one β2-microglobulin protein fragment which is selected from the group consisting of: I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

In a preferred embodiment a biomarker of the invention is used to detect transplant rejection. In another preferred embodiment, the transplant is a kidney transplant.

### Methods for Detecting β2-Microglobulin Protein Fragments

In several embodiments of the invention the methods involve the detection of β2-microglobulin protein fragments. In a preferred embodiment, β2-microglobulin protein fragments are detected using antibodies that specifically bind to β2-microglobulin protein fragments. Antibodies to β2-microglobulin protein fragments can readily be prepared by a person skilled in the art.

### Antibodies

Antibodies to β2-microglobulin protein fragments may be prepared using techniques known in the art. For example, by using a peptide of a β2-microglobulin protein fragment, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated.

The term “antibody” as used herein is intended to include fragments thereof which also specifically react with one or more β2-microglobulin protein fragments or sub-fragments thereof. Antibodies can be fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab′ fragments.

Chimeric antibody derivatives, i.e., antibody molecules that combine a non-human animal variable region and a human constant region, are also contemplated within the scope of the invention. Chimeric antibody molecules can include, for example, the antigen binding domain from an antibody of a mouse, rat, or other species, with human constant regions. Conventional methods may be, used to make chimeric antibodies containing the immunoglobulin variable region which recognizes the gene product of β2-microglobulin antigens of the invention (See, for example, Morrison et al., Proc. Natl Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, such as, but not limited to, single-chain Fv monoclonal antibodies reactive against β2-microglobulin protein fragments may also be generated by screening expression libraries encoding immunoglobulin genes, or portions thereof, expressed in bacteria with peptides produced from the nucleic acid molecules of β2-microglobulin fragments. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

Antibodies specifically reactive with β2-microglobulin protein fragments, or derivatives, such as enzyme conjugates or labeled derivatives, may be used to detect β2-microglobulin protein fragments in various samples (e.g. biological materials). They may be used as diagnostic or prognostic reagents and they may be used to detect abnormalities in the level of protein expression, or abnormalities in the structure, and/or temporal, tissue, cellular, or subcellular location of β2-microglobulin protein fragments. In vitro immunoassays may also be used to assess or monitor the efficacy of particular therapies. The antibodies of the invention may also be used in vitro to determine the level of expression of a gene encoding β2-microglobulin protein fragments in cells genetically engineered to produce β2-microglobulin protein fragments.

The antibodies may be used in any known immunoassays which rely on the binding interaction between an antigenic determinant of β2-microglobulin protein fragments and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify β2-microglobulin protein fragments in a sample in order to determine its role in transplant rejection and to diagnose transplant rejection.

In particular, the antibodies of the invention may be used in immunohistochemical analyses, for example, at the cellular and subcellular level, to detect one or more β2-microglobulin protein fragments, to localize it to particular cells and tissues, and to specific subcellular locations, and to quantitate the level of expression.

Cytochemical techniques known in the art for localizing antigens using light and electron microscopy may be used to detect β2-microglobulin protein fragments. Generally, an antibody of the invention may be labeled with a detectable substance and β2-microglobulin protein fragments may be localized in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualized by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against β2-microglobulin protein fragments. By way of example, if the antibody having specificity against β2-microglobulin protein fragments is a rabbit IgG antibody, the second antibody may be goat anti-rabbit gamma-globulin labeled with a detectable substance as described herein.

Where a radioactive label is used as a detectable substance, β2-microglobulin protein fragments may be localized by radioautography. The results of radioautography may be quantitated by determining the density of particles in the radioautographs by various optical methods, or by counting the grains.

Labeled antibodies against β2-microglobulin protein fragments may be used in identifying patients undergoing transplant rejection i.e. in imaging. Typically for in vivo applications, antibodies are labeled with radioactive labels (e.g. iodine-123, iodine-125, iodine-131, gallium-67, technetium-99, and indium-111). Labeled antibody preparations may be administered to a patient intravenously in an appropriate carrier at a time several hours to four days before the tissue is imaged. During this period unbound fractions are cleared from the patient and the only remaining antibodies are those associated with the transplant. The presence of the isotope is detected using a suitable gamma camera.

### Aptamers

The β2-microglobulin protein fragments may also be detected using nucleic acid aptamers. Aptamers are macromolecules such as RNA or DNA that can bind a specific target such as a protein or protein fragment. The three-dimensional shape of the nucleic acid allows it to bind tightly to its target. Aptamers are highly specific and can distinguish between closely related molecules and may be useful for distinguishing between β2-microglobulin protein fragments and β2-microglobulin protein. In addition they exhibit high affinity for their target and can have affinities in the picomolar to nanomolar range for proteins. Aptamers can be modified to reduce their sensitivity to enzymatic degradation and may be immobilized on a solid carrier or support as similarly described above for antibodies.

### Protease Involved in β2-Microglobulin Degradation

The inventors have characterized the protease(s) involved in fragmenting β2-microglobulin. The inventors have confirmed earlier observations (111,112), that the cleavage/degradation of urinary β2-microglobulin requires a pH<6. The responsible enzymes belong to the aspartic protease family as only pepstatin could prevent β2-microglobulin cleavages at pH 5 and most aspartic proteases have their pH optimum in the acidic range. Recently, two members of the aspartic protease family have been detected in human urine (cathepsin D (113) and napsin A (114)). Both enzymes are primarily located in lysosomes and are involved in protein degradation. Cathepsin D is found in the kidney in the distal tubules and collecting ducts (109), whereas napsin A is mainly found in the proximal tubules (115,108). The degradation of intact urinary β2-microglobulin by cathepsin D has been demonstrated, however, only two of the reported cleavage sites found by N-terminal sequencing are consistent with the 26 found in this study (113). This suggests that other aspartic proteases are involved in cleavage of urinary β2-microglobulin.

Accordingly, in one embodiment, the present invention provides a method of detecting kidney dysfunction in an animal comprising:

(a) testing a urine sample from the animal for protease activity, wherein increased protease activity when compared to a control sample indicates that the animal has kidney dysfunction.

In a preferred embodiment, the urine sample from the animal is tested for aspartic protease activity.

In another preferred embodiment, the urine sample from the animal is tested for the activity of an aspartic protease selected from the group consisting of cathepsin D and napsin A.

### Methods of Detecting β2-Microglobulin Protease Activity

The present invention also provides assays for detecting the activity of the protease(s) involved in fragmenting β2-microglobulin. These assays include assays to detect the cleavage of selected substrates (synthetic or native), for example peptide substrates bearing one or more known cleavage sites, utilizing a sample from a patient. Enzyme activity can be measured in a number of ways: (i) calorimetrically, (ii) by release of radioactive fragments, (iii) by conducting fragment analysis (gels, mass spectrometric), or (iv) immunologically, based upon the appearance or loss of reporter epitopes. Many of these methods of measurement and detection are well known in the art. Details of the specific assay would vary with the approach chosen.

### Kits

The methods described herein may be performed by utilizing pre-packaged diagnostic kits comprising the necessary reagents to perform any of the methods of the invention.

Accordingly, in one embodiment the invention provides a kit for detecting transplant related disease in an animal comprising (i) reagents for conducting a method of the invention and (ii) instructions for its use.

The kits may include at least one specific nucleic acid or antibody described herein, which may be conveniently used, e.g., in clinical settings, to monitor kidney function, to detect kidney dysfunction, and to screen, monitor and diagnose transplant recipients for transplant health or the development of transplant related disease. For example, the nucleic acid may be an aptamer that interacts with a β2-microglobulin protein fragment. The kits may also include nucleic acid primers for amplifying nucleic acids encoding protein profile distinct protein fragments in the polymerase chain reaction. The kits can also include nucleotides, enzymes and buffers useful in the method of the invention as well as electrophoretic markers such as a 200 bp ladder. The kits can also include antibodies that specifically bind β2-microglobulin or fragments thereof, and secondary antibodies for detecting those primary antibodies. The kit will also include detailed instructions for carrying out the methods of the invention.

The following non-limiting examples are illustrative of the present invention.

### EXAMPLE 1

### Methods and Materials

### Patient Characteristics

### Transplanted Patients

All patient data (e.g. allograft function measured by serum creatinine, biopsies) and urine data were stored and managed in a central access database. From July 1997 to March 2003, 2400 serial mid-stream urine samples from 212 renal transplant patients were collected. These 212 patients underwent a total of 693 protocol or clinically indicated core needle allograft biopsies. All patient charts were reviewed and additional information extracted as needed. Biopsies were analysed by experienced renal pathologists, and scored according to the Banff 1997 classification (Table 3) (23). The acute Banff score determines acute interstitial (ai 0-3), tubular (at 0-3), vascular (av 0-3) and glomerular (ag 0-3) changes, whereas the chronic Banff score assesses chronic interstitial (ci 0-3), tubular (ct 0-3), vascular (cv 0-3) and glomerular (cg 0-3) changes. The individual scores are added to a total acute (a 0-12) and total chronic (c 0-12) score. A biopsy specimen was judged adequate, when ≧7 glomeruli and ≧1 vessel were available for analysis. All patients were treated with a triple immunosuppressive regimen consisting of calcineurin-inhibitor (cyclosporine or tacrolimus), prednisone and mycophenolate-mofetil or azathioprine.

### Non-Transplanted Control Groups

[1] Normal control group: Consists of 28 healthy individuals (14 female and 14 male, age 20-50 years).

[2] Urinary tract infection (UTI) group: Consists of 5 females with an episode of a lower UTI, which was defined as requiring the clinical symptoms of a UTI, a leukocyte count in the urine sediment >40 per high power field and a positive bacterial culture (>10⁸ colony forming units).

### Urine Collection, Preparation and Microscopic Analysis

### Urine Collection and Storage for Evaluation of SELDI-TOF-MS Platform

Second-morning urine from healthy men and women were collected in two different containers. The first 10-20 mL of urine collected was considered first-void urine, the following 50-80 mL mid-stream urine. Urines were centrifuged in a fixed angle centrifuge for 10 minutes at 2000 rpm (900 g), the supernatants were transferred into 2 mL cryo-tubes (Gordon Technologies Inc., Missisauga, ON) and stored at −80° C. until further analysis. All samples were obtained with informed consent and ethics approval of the University of Manitoba Institutional Review Board. For urine sediment analysis 10 mL of freshly collected urine was centrifuged for 10 minutes at 2000 rpm. The pellet was analyzed with a phase-contrast microscope at 400× magnification and is reported as cells per high power field (hpf).

### Urine Collection in Transplanted Patients and Non-Transplanted Control Groups for Biomarker Discovery

All urine samples were stored non-centrifuged at −80° C. until further analysis. All transplanted patient and control group urine samples were obtained with informed consent and ethics approval by the University of Manitoba institutional review board.

### Mass Spectrometry—SELDI-TOF-MS

Urine samples were thawed on ice, shortly vortexed and centrifuged for 5 minutes at 10000 rpm (to remove remaining cell particles). Two different ProteinChips were used for the analysis. They were prepared as follows:

[1] Normal phase chips (ProteinChip NP20; Ciphergen, Freemont, Calif.): Five μL of urine supernatant were applied in duplicate to the chip and incubated for 20 minutes in a humidity chamber. Spots were then washed three times with 5 μL HPLC-grade water and air-dried for 10 minutes.

[2] Hydrophobic chips (ProteinChip H4): Five μL of 50% acetonitrile in HPLC-grade water were applied to the spots for 5 minutes to activate the surface. This solution was removed and 5 μL urine supernatant were applied in duplicate to the chip and incubated for 20 minutes in a humidity chamber. Spots were washed twice with 5 μL 10% acetonitrile in HPLC-grade water and then once with 5 μL HPLC-grade water. Chips were air-dried for 10 minutes.

As matrices saturated α-cyano-4-hydroxycinnamic acid (CHCA: Ciphergen) and sinapinic acid (SPA: Ciphergen) were prepared in 50% acetonitrile/0.5% trifluoro-acetic acid (TFA) according to the manufacturer's instructions and 1 μL of matrix solution (35% CHCA unless otherwise specified) was applied to each spot and air-dried. Unless stated otherwise, chips were read with the following SELDI-TOF-MS instrument (ProteinChip Reader II: Ciphergen) settings in the positive ion mode: Laser intensity 230; detector sensitivity 6; detector voltage 1800 V; positions 20 to 80 were read with an increment of 5 (resulting in 13 different sampling positions); sixteen laser shots were collected on each position (total shots collected and averaged: 208/sample); eight warming shots were fired at each position, which were not included in the collection; the acquired mass range was from a mass-over-charge (m/z) ratio of 0 to 80000; lag time focus of 900 ns. Calibration was done externally with a mixture of 4 proteins with masses ranging from 2 to 16 kDa. After baseline subtraction, peak labeling was performed by the ProteinChip Software (Version 3.1) for peaks with a signal-to-noise (S/N) ratio of ≧3 in the m/z range from 2000-80000. For some comparisons and presentations spectra were normalized according to the total ion current.

### Protein Purification and Identification Methods

### Determination of Point of Iso-Electricity (pI) of Rejection Pattern Proteins

A urine sample with the rejection pattern proteins was dialysed with 7 kD cut-off dialysis cassettes (Slide-A-Lyzer, Pierce, Rockford, Ill.) against 50 mmol/L MES pH 6 and 50 mmol/L Tris pH 8, respectively. Cation-exchange (CM HyperD, Ciphergen) and anion-exchange (Q HyperD, Ciphergen) beads were washed three times for 20 minutes with 1 mL 50 mmol/L MES pH 6 or 50 mmol/L Tris pH 8, respectively. The pH 6 fraction was incubated on CM-beads for 2 h in a ratio of 5 μL beads per 1 mL urine. The supernatant was transferred to a separate tube. After washing the CM-beads twice with two bead-volumes 50 mmol/L MES pH 6 for 15 minutes, proteins were eluted with increasing concentrations of KCl in 50 mmol/L MES pH 6 (two bead-volumes for 30 minutes each). The supernatant and the eluted fractions were checked for the presence or absence of the rejection pattern proteins by SELDI-TOF-MS. The pH 8 fraction was incubated on Q-beads for 2 h in a ratio of 5 μL beads per 1 mL urine. The supernatant was transferred to a separate tube. After washing the Q-beads twice with two bead-volumes 50 mmol/L Tris pH 8 for 15 minutes, proteins were eluted with increasing concentrations of NaCl in 50 mmol/L Tris pH 8 (two bead-volumes for 30 minutes each). The supernatant and the elution fractions were checked for the presence or absence of the rejection pattern proteins by SELDI-TOF-MS.

Purification of Rejection Pattern Proteins with Cation Exchange (CM) Beads and Reverse-Phase High-Pressure Liquid Chromatography (RP-HPLC)

Fifteen mL of urine sample with the rejection pattern proteins was dialysed with 6-8 kD cut-off dialysis tube membrane (Spectra/Por, Spectrum Laboratories, Rancho Dominguez, Calif.) against 50 mmol/L MES pH 6.2. Dialysed urine was transferred into 1.5 mL siliconized tubes (Fisherbrand) and previously washed CM-beads (see above) were added in a ratio of 5 μL beads per 1 mL urine. After 2 h incubation the supernatant was transferred to a separate tube and the CM-beads were washed twice with two bead-volumes 50 mmol/L MES pH 6.2 for 15 minutes. Proteins were eluted with two beadvolumes 200 mmol/L KCl in 50 mmol/L MES pH 6.2. Those fractions containing the rejection pattern proteins were lyophilized and resuspended in a 5 times smaller volume of HPLC-grade water.

Further purification was done by RP-HPLC using an Agilent 1100 Series with a C4 column (Zorbax SB-C4, 5 μm, 0.5×150 mm; Agilent Technologies, Paulo Alto, Calif.). Five μL of concentrated sample was applied and eluted using a 1.6% acetonitrile increment per minute in 0.1% TFA during the first 17 minutes, followed by a 0.3% increment per minute for 24 minutes and a 16% increment per minute for the last 4 minutes at a flow rate of 20 μL/minute. Peak fractions containing the rejection pattern proteins were pooled, lyophilised and resuspended in 50 mmol/L ammonium bicarbonate for in solution digestion. The purification process was monitored with SELDI-TOF-MS using H4 chips.

### Identification of Rejection Pattern Proteins by Liquid-Chromatography Mass Spectrometry (μLCMS) and Tandem Mass Spectrometry (μLC-MS/MS)

Concentrated and purified protein (from about 10 mL starting material) was reduced with 10 mM DDT for 30 minutes at 57.5° C., alkylated with 50 mM iodoacetamide for 30 minutes in the dark, then dialysed against 50 mmol/L ammonium bicarbonate, and finally digested with 100 ng trypsin (sequencing-grade modified trypsin, Promega) over night at 37° C. Peptides were lyophilised, resuspended in 5 μL 0.1% TFA, and subjected to RP-HPLC separation using an Agilent 1100 Series system with a C18 column (Vydac 218 TP C18, 5 μm, 0.15×150 mm). Peptides were eluted with a linear gradient of 1.3% acetonitrile increment per minute in 0.1% TFA during 35 minutes and a 10% increment for the last 5 minutes. The column effluent (4 μl/min) was mixed online with 2,5-dihydroxybenzoic acid (0.16 g/ml, Sigma-Aldrich) matrix solution (0.5 μl/min) and deposited by a small computer-controlled robot onto a movable MALDI target at one-minute intervals. Forty such fractions were collected over a total period of 40 minutes. The spots on the target were analyzed individually, both by single mass spectrometry (MS) and by tandem mass spectrometry (MS/MS) in the Manitoba/Sciex prototype quadrupole/time-of-flight mass spectrometer (QqTOF) (81). In this instrument, ions are produced by irradiation of the target with photon pulses from a 20-Hz nitrogen laser (Laser Science) with 300 mJ energy per pulse. Orthogonal injection of ions from the quadrupole into the TOF section normally produce a mass resolving power 10,000 FWHM and accuracy within a few mDa in the TOF spectra in both MS and MS/MS modes, as long as the ion peak is reasonably intense. MS and MS/MS peak list were submitted to Profound and searched against the non-redundant NCBI human database using a mass accuracy of 20 ppm of monoisotopic peaks. Partial methionine oxidation and one trypsin miscleavage was allowed.

### Determination of Cytomegalovirus (CMV) Viremia

CMV-viremia was measured on peripheral blood buffy coat specimens using a semi-quantitative PCR assay developed at the Manitoba Cadham Provincial Laboratory that is accredited by the College of American Pathologists.

### Statistical Analysis

JMP IN software version 4.0.4 (SAS Institute Inc., Cary, N.C.) was used for statistical analysis. For categorical data, Fisher's exact test or Pearson's chi-square test was used. Parametric continuous data was analyzed by Student t-tests or one-way analysis of variance. For nonparametric continuous data, Wilcoxon or Kruskal-Wallis rank sum tests were used. A P-value <0.05 (two-sided test) was considered to indicate statistical significance.

### EXAMPLE 2

### Urine Protein Profiling

Healthy people secrete less than 150 mg of protein in urine each day. Depending on the kidney or urinary tract system disease, proteinuria can reach more than 10 g per day. Basically, there are four different pathophysiological pathways that influence the protein content and composition of urine.

[I] Filtration from serum: The major part of urine proteins is derived from serum by filtration through the glomerular barrier. The glomerular barrier consists of the fenestrated endothelial cells, the glomerular basement membrane and the slit-diaphragm of the podocytes. The latter is considered to be predominantly responsible for the characteristics of the barrier. Proteins are thought to be retained from filtration into the urine based on their molecular weight, size, shape and net charge (75). Normally, proteins below 20 kDa are completely filtrated into urine, whereas larger proteins are generally retained in the serum. Albumin (66 kDa), for instance, would still pass the glomerular barrier based on its size, but it is speculated that its negative charge prevents filtration of large amounts. However, not everyone is in agreement with this hypothesis of charge selectivity (76).

[II] Tubular reabsorption and regurgitation: Many filtrated proteins bind to more or less specific receptors mainly on proximal tubular epithelial cells (e.g. megalin and cubilin). After binding, ligands are trafficked to lysosomes for degradation or endocytic vesicles for transcytosis back to the blood stream (77). Lysosomal degraded proteins may be directed back to the blood stream, but they are also regurgitated into the tubular lumen and ultimately excreted. The latter pathway was not recognized until recently and may have been underestimated (76,78). It is critical to take this pathway into account for proteomic analysis in urine, because not only intact proteins but also fragments of the same protein may be detectable.

[III] Active secretion: Some proteins are produced and secreted from tubular cells into the urine by an active process (e.g. Tamm-Horsfall protein) (79). Even whole vesicles can be released. Furthermore, cells with access to the urinary tract system can secrete proteins into it (e.g. neutrophils secrete α-defensins).

[IV] Cell-death derived proteins: Tubular cells undergo constant renewal and ‘old’/apoptotic cells are shed into the urine. Prescott estimated that, under physiological conditions, almost 2,000,000 tubular epithelial cells are sloughed into the urine each day (80). In addition, red and white blood cells as well as urothelial cells can be present in urine in significant amounts. Cells may stay intact or their membranes may rupture, releasing intracellular proteins into the urine.

### Proteomics Versus Genomics: Advantages and Disadvantages

The decoding of the human genome, developments in microtechnology, bioinformatics and mass spectrometry made it possible to investigate complex biological processes on a broad gene and protein level. Gene-microarrays (38,65) and MS-based proteomics (66,67) have gained widespread applications in biomedical research, including identification of candidate genes/proteins for diagnostic, prognostic and therapeutic purposes. However, both approaches have their limitations, which are mainly related to the technology itself (Table 1).

### Choosing a Platform for Urine Protein Profiling

### Properties and Limitations of Proteomic Technology

At present, there are several techniques to identify and compare the expression of proteins, each with advantages and disadvantages (Table 2). The most established method is protein separation by two-dimensional gel-electrophoresis (2-DE) followed by in-gel digestion and peptide mass fingerprinting by mass spectrometry. This method allows for the comparison of the relative abundance of proteins. However, there are several limitations of 2-DE as a separation method for proteomic studies. The resolvable range of molecular weights is limited at both ends, with a bias toward high abundance proteins. In addition, the technique requires relatively large amount of sample, is labour-intensive, and good gel-to-gel reproducibility can be hard to achieve (68,69). Thus, this approach is not optimal for high-throughput profiling.

An alternative approach uses one- or two-dimensional liquid chromatography as the separation step upstream from the mass spectrometer (liquid chromatography coupled to mass spectrometry, μLC-MS). While this technique provides information about the protein content of the samples, little information about their relative abundance can be obtained, unless the proteins/peptides are labelled first by isotope-coded affinity tags (70,71) or other protein/peptide labelling techniques (e.g. digestion with H2160 and H2180 mixture (72,73,74)). Furthermore, this method is still labour-intensive and has limited throughput. Surface-enhanced laser desorption/ionization time-of-flight mass-spectrometry (SELDI-TOF-MS) addresses some of the limitations of both 2-DE and μLC-MS. It combines matrix-assisted laser-desorption/ionization time-of-flight mass spectrometry (MALDI-TOF-MS) to surface chromatography.

Specifically, a sample is applied to a chip surface carrying a functional group (e.g. hydrophobic, anion-exchange, cation-exchange, normal phase and metal-affinity). After incubation, proteins that do not bind to the surface are removed by a simple wash step, and bound proteins are analysed by mass spectrometry. This approach, in contrast to the others described, allows for high-throughput profiling of many clinical samples, but has limited sensitivity, resolution and mass accuracy.

### Advantages and Limitations of SELDI-TOF-MS for Urine Protein Profiling

SELDI-TOF-MS offers many advantages for protein profiling in urine. First, only 5 to 10 μL of sample is needed for one analysis. Second, due to the simple chip preparation, many samples can be analyzed quickly. Third, the washing step removes most of the salts, which otherwise interfere with mass spectrometric analysis. And fourth, the impact of different chromatographic chemistries can be analyzed, which may allow one to find optimal purification conditions for a protein of interest in a short time with small amounts of sample. However, sensitivity is moderate (especially in a complex mixture), and resolution above 25 kDa is low, resulting in a limited part of the urine proteome been detectable by SELDI-TOF-MS. In addition, standardization of analysis conditions is essential, and both extrinsic and intrinsic factors must be taken into account for accurate data interpretation.

Finally, protein microarrays, consisting of thousands of protein-specific capturing molecules (e.g. antibodies) in analogy to gene-microarrays, may revolutionize protein expression profiling. However, the few currently available antibodies largely limit this technology.

Urine Protein Profiling with SELDI-TOF-MS

### Evaluation of Reproducibility

In order to be able to compare the proteome of many samples, a high-throughput platform is mandatory. SELDI-TOF-MS system is a high-throughput platform available. Reliable profiling of clinical samples, required the reproducibility and the limitations of the SELDI-TOF-MS platform to be determined. In addition, several intrinsic (e.g. urine concentration, cellular components) and extrinsic (e.g. stability of urine proteins, storage) factors of urine were studied to confidently attribute differences in protein composition in various disease states to the disease process itself and not to confounding factors.

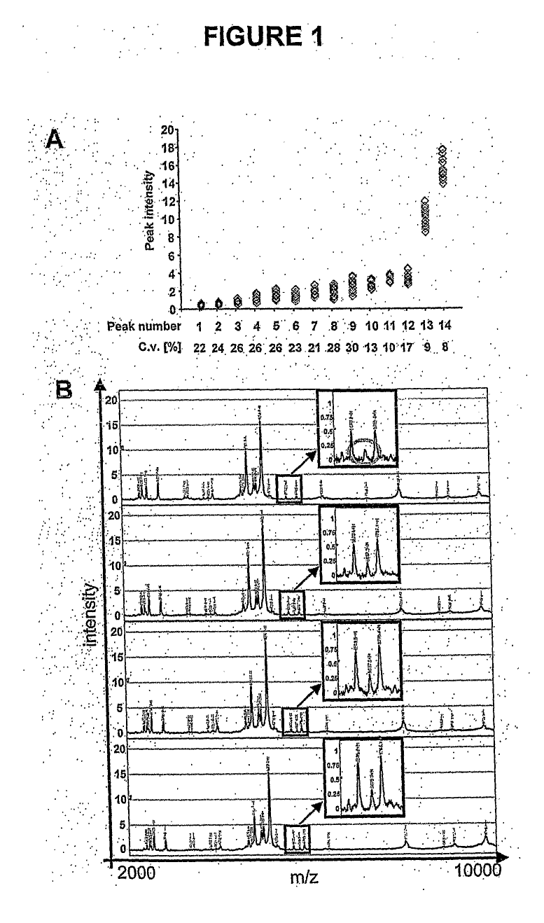

Reproducibility was evaluated by applying one urine sample to 14 spots and reading the spots using the protocol described in Example 1. The total number of detected peaks with an S/N-ratio ≧3 was 25 peaks/spectrum (range 23-29). Fourteen peaks common to all spectra were selected and compared with regard to their peak intensity by calculating the coefficient of variation. They ranged from 8 to 30%, with the lowest variation seen in the high intensity peaks and the higher variation seen in lower intensity peaks (FIG. 1A). This is expected, as small differences in low intensity peaks (e.g. 1.0 vs. 0.5) have a large influence on the calculated coefficient of variation. Independent of the software assignment of protein peaks, it is important to conduct manual inspection of the spectra, to determine whether a specific peak is present. Low intensity peaks with a S/N-ratio near the selected detection-threshold (i.e. ≧3) can be unlabelled and undetected by the software (FIG. 1B).

### EXAMPLE 3

### Impact of Extrinsic Factors on Reproducibility and Peak Detection of Urine Protein Profiles

The most important extrinsic factors that influence reproducibility and peak detection are the matrix composition and the instrument settings. Matrix allows for efficient ionization and vaporization of proteins (82). The most popular matrices for the SELDI-TOF-MS system are SPA and CHCA. Saturated SPA is preferable for looking at masses above 10-20 kDa, while 10-20% CHCA provides the best resolution for proteins/peptides up to about 5 kDa. For urine protein profiling from 2-25 kDa, more peaks and a higher degree of resolution were observed with 35% CHCA. Instrument settings such as detector sensitivity, detector voltage, and laser intensity have to be determined individually. The higher the detector sensitivity and voltage or the laser intensity, the better the detection of high mass proteins. This is accompanied by an increase in background noise, which limits detection of low intensity peaks. The impact of matrix on the urine protein profile was determined by comparing different dilutions of CHCA and SPA (20%, 35%, 50% and 100%) with the otherwise unchanged protocol stated above. In the range from 2-25 kDa, 22, 26, 19 and 16 peaks were detected using 20%, 35%, 50% and 100% CHCA, respectively. In contrast, 13, 19, 11 and 10 peaks were detected using 20%, 35%, 50% and 100% SPA. Peak intensity below 8-10 kDa was higher with CHCA, whereas SPA yielded higher peak intensities above 8-10 kDa (urine protein profiles not shown).

The impact of spot sampling protocols was determined by comparing three different spot sampling protocols with respect to peak detection in undiluted and diluted urine: protocol 1 (standard protocol; see reference 96); protocol 2 (standard protocol modified to sample on only 5 different positions for a total of 80 shots/sample); protocol 3 (standard protocol modified to use a higher detector sensitivity (10 instead of 6)). Protocol 1 detected 34 peaks in undiluted urine, whereas protocols 2 and 3 detected only 21 and 26 peaks, respectively. In diluted urine (urine creatinine 3.75 mmol/l) the peak counts were 20, 11 and 13, respectively (urine protein profiles not shown).

The number of positions sampled on a spot is an important parameter for optimal peak detection. Ideally, all proteins are distributed homogeneously on the chip and are crystallized homogeneously in the matrix. If so, one would expect to generate the same spectra at every position. From the three spot sampling protocols it is clear, that there are ‘hot positions’, where proteins are clustered on the spot leading to the detection of an abundance of peaks with a high intensity. Similarly, there are ‘cold positions’, where only few or even no peaks are detected. Unfortunately, ‘hot position’ sampling may not accurately profile low abundant proteins due to ion suppression that can occur due to high abundance proteins. Therefore, the most representative spectra for a given urine sample is achieved by sampling many different spot positions and combining the data. This is especially true for dilute urine samples.

If the SELDI-TOF-MS approach is to be used in the assessment of clinical samples, it is important to assess the stability of the urine proteins prior to analysis. Recent studies have found little or no changes in albumin, retinol-binding protein, N-acetyl glucosaminidase, IgG and kappa/lambda light chain concentrations after storage at room temperature, 4° C., −20° C. and −70° C. (83,84,85,86).

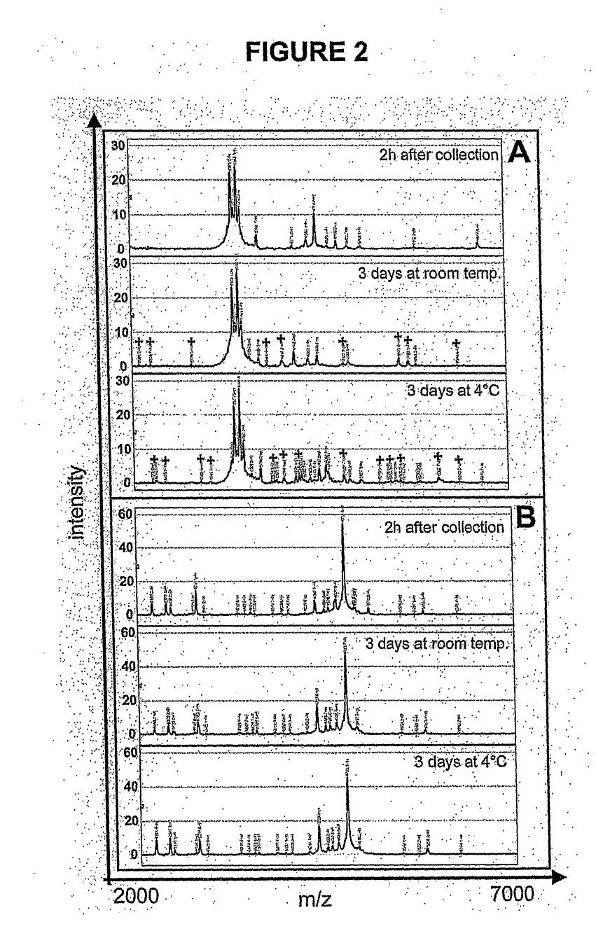

First-void and mid-stream urine samples from three females and three males were analyzed within 2 hours from the time of collection, after storage for three days at room temperature and after three days at 4° C. In all six samples, only minor differences in the mid-stream urine protein profiles could be detected. However, in three first-void urines (two female, one male), storage for three days at room temperature or at 4° C. changed the spectra. A series of new peaks in the low molecular weight range was detected (FIG. 2). First-void urine can have significant bacterial contamination resulting in either urine protein degradation and/or contamination with bacterial proteins within a few days.

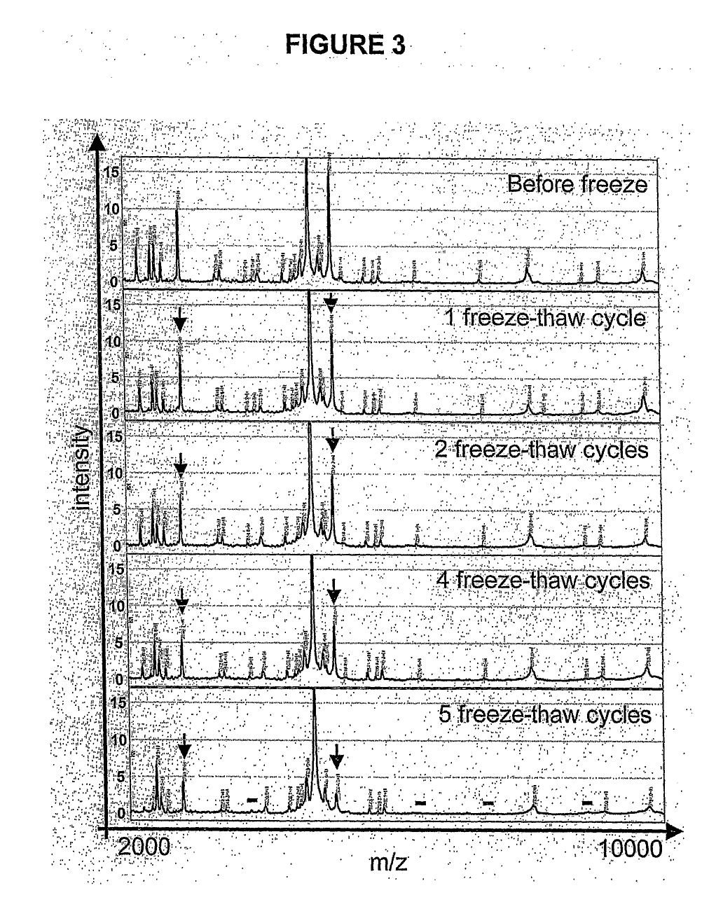

Storage of the urine samples at −70° C. did not change the spectra compared to those obtained before freezing. Furthermore, almost the same spectra could be generated after four freeze-thaw cycles, however, a loss of peaks was observed after the fifth freeze-thaw cycle (FIG. 3).

### Impact of Intrinsic Factors on Normal Urine Protein Profiling

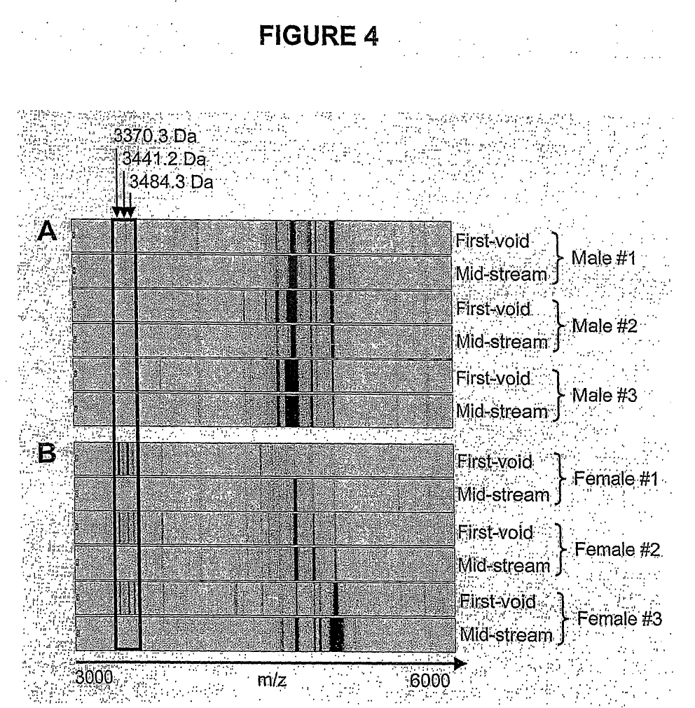

Mid-stream urine is the standard for almost all urine analysis. In a clinical setting, there are always urine samples that are not mid-stream urines. Therefore, knowing the variation in urine protein profiles that may occur between first-void and mid-stream urines is important. In all three urine samples from males, there are almost no differences between the protein profile of first-void and mid-stream urine (FIG. 4A). However, in all three urine samples from females, there are prominent peaks between 3.3 and 3.5 kDa in the first-void urine fraction. These peaks are greatly diminished in the mid-stream urine sample, together with other changes in peak intensities (FIG. 4B). Three of these peaks with average masses of 3370.3, 3441.2 and 3484.3 Da are consistent with the masses of the α-defensins 2, 1, and 3, respectively (Swiss Prot P59665+P59666; 3371.9, 3442.5, 3486.5 Da)). Indeed, α-defensins, which are an important part of the human antimicrobial defense (87,88), have been detected by SELDI-TOF-MS technology in urine (89), as well as in culture supernatants of human CD8+ T-cells (90). The differences in the protein profile between first-void and mid-stream urines may be explained by urethral secretion of these proteins, which are then washed away by the first-void urine. Therefore, consistent urine protein profiling requires mid-stream urine samples for analysis, because first-void urine has a different protein composition than mid-stream urine and is more prone to protein degradation.

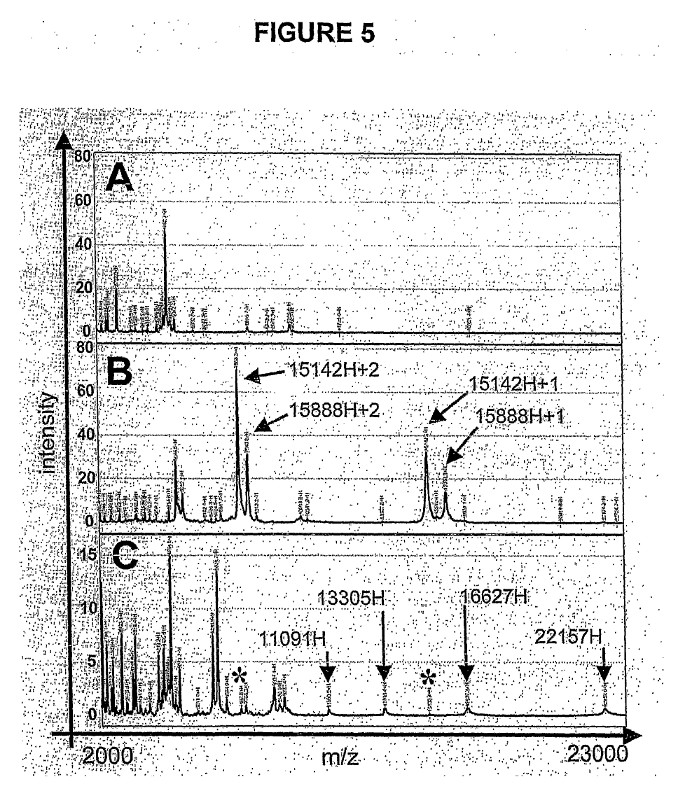

Another confounding variable in urine proteomic analysis is the presence of blood in urine. It can be present in urine under normal conditions (e.g. menstruation) or in association with urogenital tract pathologies. To investigate the impact of blood on the normal urine profile (FIG. 5A), 500 μL urine was spiked with 10 μL of blood, which resulted in a red colouring of the sample (sediment analysis showed >100 red blood cells (RBC)/hpf). In the subsequent analysis by SELDI-TOF-MS, four major peaks were detected (FIG. 5B), which are consistent with the masses of the hemoglobin α- and β-chains and their doubly charged ions (Swiss Prot P01922: 15126 Da; P. Based on the virtual disappearance of these peaks after sample centrifugation prior to SELDI-TOF-MS analysis, it is likely that these peaks represent hemoglobin. They were easily detectable as the most intense peaks up to a 1:128 dilution of this sample, corresponding to 10 μL blood in 64 ml diluted urine (urine protein profiles not shown). However, even when the RBC were removed by centrifugation, the urine was still contaminated with serum proteins. This is suggested by the presence of peaks with masses consistent with albumin in the urine protein profile (FIG. 5C). Albumin has a molecular weight of 66472 Da with its multiply-charged ions at an m/z of 33236 (double-charged), 22157 (triplecharged), 16618 (quadruple-charged), 13294 (quintuple-charged) and 11079 (sextuple-charged).

Blood was observed to be a major confounding variable affecting the normal urine protein profile. Not only did new peaks appear (i.e. peaks consistent with the masses of hemoglobin and albumin), but many of the normal peaks observed became undetectable. This is likely due to ion suppression by the blood proteins. Notably, even with a dilution of 10 μL blood in 64 ml diluted urine (1:6400 dilution), the peaks consistent with hemoglobin remained dominant. Clearly, such contamination invalidates any interpretation of the urine protein profile. Although centrifugation of the urine sample removes RBC, contamination with serum proteins will still continue to confound the urine protein profile

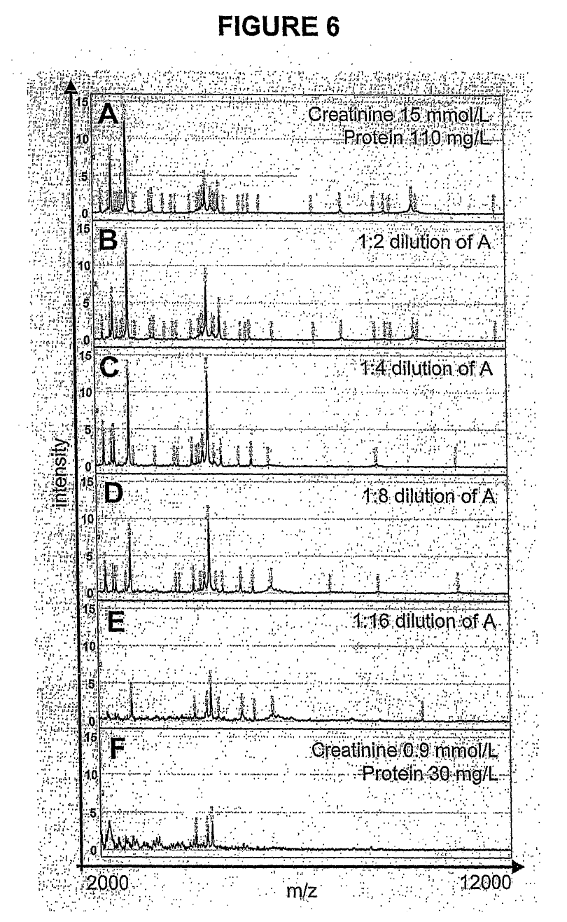

A dilute urine sample may limit the ability to detect the normal urine protein profile. To address the issue of urine concentration, urine was sampled from a healthy male person with a body weight of 75 kg after 20 hrs of no fluid intake. The measured urine creatinine was 15 mmol/L and the total protein was 0.11 g/L. At another time point, the same individual was challenged with 4 L of fluid over 2 hrs, leading to dilute urine with a creatinine of 0.9 mmol/L and a total protein of 0.03 g/L. While the concentrated urine showed the normal peak profile (FIG. 6A), the dilute urine sample showed only three peaks in the range from 2-25 kDa (FIG. 6E). To determine the detection threshold of the normal urine profile, the concentrated urine sample was serially diluted (FIG. 6B-6E). At a 1:2 dilution, which corresponds to a urine output of 2 L/day (calculated in our test person by: creatinine production/day [0.2 mmol/kg/day*75 kg] divided by urine creatinine [7.5 mmol/L]=2 L/day) the profile remained unchanged (FIG. 6B). A progressive loss of urine profile peaks started with a 1:4 dilution. The 1:16 dilution showed a spectrum similar to the urine profile obtained after the fluid challenge (FIG. 6E).

Depending on fluid intake the kidneys can concentrate urine to an output as low as 0.5 L/day, or dilute urine to almost 20 L/day. Under normal conditions, about 1-2 L urine are excreted per day. In a very dilute urine sample (urine creatinine 0.9 mmol/L), most of the proteins could not be detected on a NP20-chip. The threshold for a stable urine protein profile on a NP20-chip was a urine output of 2 L/day. Because every ProteinChip type has different binding capacities, the detection threshold has to be determined for every chip type individually.

### EXAMPLE 4

### Protein Quantification and Detection Limits with SELDI-TOF-MS

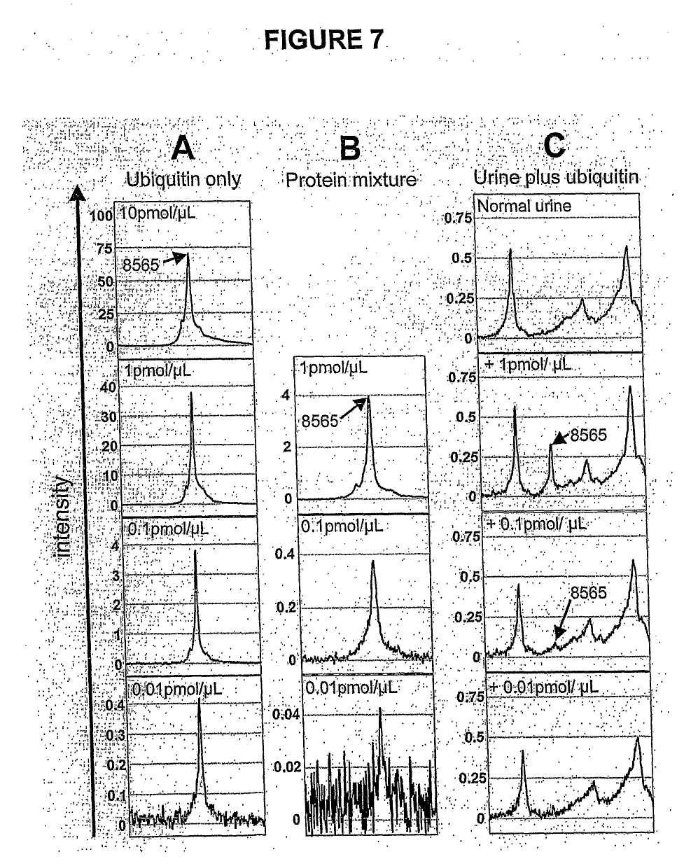

Peak height and area under the peak have been used to reflect protein abundance (89,91). To determine if either the spectral peak intensity or area provides a means for reliable protein quantification, serial dilution of a single protein (ubiquitin, 8565 Da) was performed. There was an excellent correlation between the amount of protein in the sample and peak intensity (r₂=0.95) or the area under the peak (r₂=0.98) in non-normalized spectra (FIG. 7A). Even in a mixture containing four other proteins, the correlation was maintained (r₂=0.99 for peak intensity and for the area under the peak), but the peak intensities were 10 times lower with the same amount of ubiquitin (FIG. 7B). When a complex protein mixture (i.e. normal urine with a protein concentration of 110 mg/L) was spiked with 1.0, 0.1 and 0.01 pmol/μL ubiquitin, only the first two concentrations of ubiquitin were detectable (FIG. 7C). The peak intensity dropped from 0.32 (1.0 pmol/μL) to 0.09 (0.1 pmol/μL), which is only a 3.5 times decrease instead of the expected 10 times. Because only two measurements of peak intensity were obtainable, no correlation was calculated. Although good correlation between the amount of a single protein alone or in a mixture with four other proteins and the peak intensity was found, it is questionable whether this remains true in a complex protein mixture (e.g. urine) due to many factors like ion suppression and competition for binding sites on the ProteinChips. Therefore, care should be taken in comparing relative peak heights between two different urine protein profiles as an indicator of change in protein abundance under different circumstances (i.e. normal versus pathologic state).

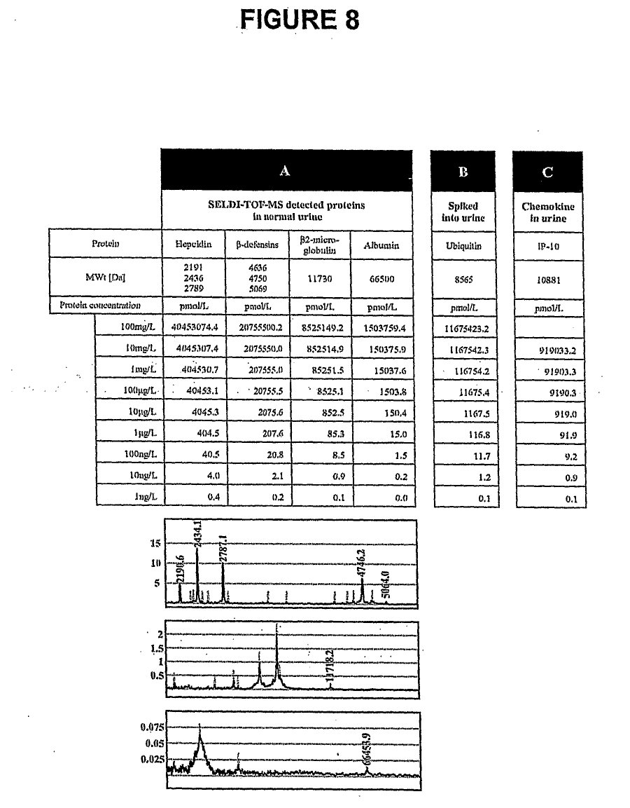

Referring now to FIG. 8, this experiment also showed that the detection limit for ubiquitin, spiked in urine, is 0.01 pmol/μL or 10000 pmol/L, respectively (FIG. 8B). Other experimental evidence for the detection limit of SELDI-TOF-MS can be extracted from dilution experiments. Several urine proteins (i.e. hepcidin at 2191, 2436 and 2789 Da; α-defensins at 4636, 4750 and 5069 Da; β2-microglobulin at 11730 Da; albumin at 66500 Da) are readily detectable in urine from a healthy individual by SELDI-TOF-MS. Serial dilution of this sample has demonstrated that all these proteins are no longer detectable starting at a 1:4 and ending at 1:16 dilution. Because the concentration of these proteins is known, the detection threshold can be approximated (FIG. 8A). The detection threshold for hepcidin is about 40 pmol/L; for β-defensins about 200 pmol/L; for β2-microglobulin about 8500 pmol/L; and for albumin about 15000 pmol/L. The detection threshold is approximately 10 times below the concentration of these proteins.

To illustrate the importance of knowing the detection threshold, an example with the chemokine IP-10 in urine is described. Normal values measured by ELISA are 1-20 ng/L; during allograft rejection concentrations up to 1 μg/L have been reported (60). Even the later concentrations are 100-1000 times below the anticipated detection threshold of 100 μg-1 mg/L based on the experimental evidence from proteins in the same molecular weight range (ubiquitin and β2-microglobulin).

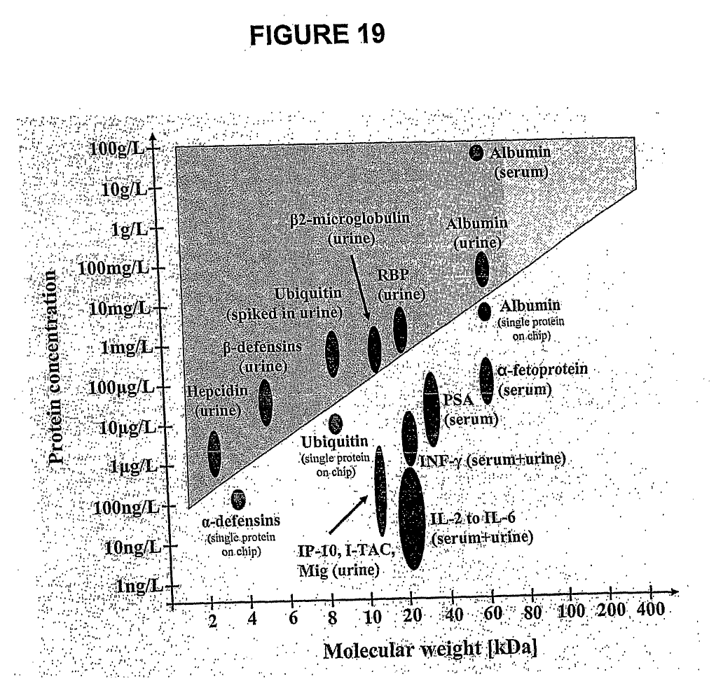

The detection of a protein by SELDI-TOF-MS is critically determined by its concentration in the sample, its binding to the chromatographic surface and its ionization process within the mass spectrometer. For single proteins, the detection threshold for α-defensins (3371 Da) was 10-100 ng/L (89), for ubiquitin (8565 Da) was 100 ng/L to 1 μg/L, and for albumin (66500 Da) was 1-6 mg/L, respectively. The increased detection threshold for high molecular weight proteins is well known and thought to be related to inferior ionization of large proteins. In a complex protein mixture (e.g. urine, serum), however, the detection threshold increases by roughly 10-1000 fold compared to the detection threshold for single proteins. This decrease in sensitivity is mainly caused by competition for binding sites (i.e. binding competition) on the ProteinChips and competition for ionization (i.e. ion suppression). Whereas the former is distinct to the SELDI-TOF-MS platform, the later is a common problem for all mass spectrometers. By changing the conditions for protein binding to different chromatographic surfaces, some proteins may be selected and enriched, whereas others may be excluded, allowing the detection limit to drop. However, the detection limit might be at best 10 times above the detection threshold for a single protein. Based on these experiments, the potentially detectable urine proteins by SELDI-TOF-MS can approximately be defined by their concentration and their molecular weight (FIG. 19). This ‘accessible’ part of the proteome becomes even smaller when the inferior sensitivity of the SELDI-TOF-MS system for proteins above 25 kDa is taken into account. Therefore, profiling strategies involving clinical samples where potential biomarkers are at a concentration and in a molecular weight range detectable by SELDI-TOF-MS may be more successful. Specifically, urine protein profiling using SELDI-TOF-MS may be sensitive enough to detect potential biomarkers in kidney diseases, because the affected cells ‘drain’ selectively into urine. In contrast, several groups use SELDI-TOF-MS for serum protein profiling in order to detect new biomarkers for early cancer detection (92,93). However, serum consists of a few high abundance proteins that account for 99% of the total protein amount (94), which may increase the detection threshold of SELDI-TOF-MS even above the one outlined for urine. It is therefore not surprising that identified potential cancer biomarkers found by SELDI-TOF-MS were all in concentrations ranging from mg/L to g/L (95), representing more likely cancer epiphenomena (e.g. liver metabolism changes) than specific cancer related proteins. In addition, it seems unlikely that a small tumor, which weighs only a few grams, produces proteins detectable in the mg/L to g/L range in serum. This is underscored by the serum protein concentration range of currently used cancer biomarkers, which is in the 0.1-100 μg/L range (95).

### EXAMPLE 5

### Detection of Urine Proteins Associated with Acute Renal Allograft Rejection

### Patient Characteristics

From July 1997 to March 2003, 2400 serial mid-stream urine samples from 212 renal transplant patients were collected. These 212 patients underwent a total of 693 protocol or clinically indicated core needle allograft biopsies. Based on allograft function, the clinical course and the allograft biopsy result, four rigidly defined patient groups were extracted from the whole patient population (n=212) as follows:

[1] Stable transplant group: Consists of 22 mid-stream urine samples (from 22 patients) obtained immediately before a protocol renal allograft biopsy performed within the first 12 months post-transplant. None of these patients had experienced DGF. All had stable allograft function (i.e. serum creatinine within 110% of baseline value at the time of biopsy), and none experienced a clinical or protocol biopsy-proven rejection prior to the date of examination. All biopsies met the criteria for adequacy and all were required to have an acute and chronic Banff score of zero (i.e. ai0t0v0g0 and ci0t0v0g0).

[2] Acute clinical rejection group: Consists of 18 mid-stream urine samples (from 18 patients) obtained immediately before a renal allograft biopsy performed within the first 12 months posttransplant. All experienced an elevation in creatinine >110% from baseline and the diagnosis of acute rejection required an acute Banff score ≧ai2t2v0g0. Patients with a chronic Banff score >ci1t1v0g0 were excluded in order to avoid chronic allograft nephropathy as a confounding variable in the analysis.

[3] Acute tubular necrosis (ATN) group: Consists of 5 mid-stream urine samples (from 5 patients) obtained immediately before a renal allograft biopsy performed within the first 6 days posttransplant to diagnose the cause of delayed graft function (DGF), which was defined as the need for hemodialysis within the first week or a drop of serum creatinine <50% from pre-transplant levels by day 5 post-transplant. Antibody mediated rejection was excluded based on a negative flow-crossmatch, and histological changes on the biopsy consistent with ATN. In all biopsies, the acute Banff score was ai0t0v0g0 and significant donor pathology was excluded by requiring a chronic Banff score of ≧ci1t1v0g0.

[4] Recurrent (or de novo) glomerulopathy group: Consists of 5 mid-stream urine samples (from 5 patients) obtained immediately before a renal allograft biopsy performed to diagnose the cause of proteinuria (≧1.5 g/day). The patients had diagnoses of membranous glomerulonephritis (GN), focalsegmental glomerulosclerosis or IgA-nephropathy and all had acute Banff scores ≦ai1t1v0g0. The acute clinical rejection group had more HLA-mismatches and a higher mean serum creatinine level at the time of the renal allograft biopsy compared to the stable transplant group. Otherwise, there were no significant differences between these groups (Table 4).

### Non-Transplanted Control Group Characteristics

Normal control group: Consists of 28 mid-stream urine samples from 28 healthy individuals (14 female and 14 male, age 20-50 years).

Urinary tract infection (UTI) group: Consists of 5 mid-stream urine samples from 5 females obtained during an episode of a lower UTI, which was defined as requiring the clinical symptoms of a UTI, a leukocyte count in the urine sediment >40/high power field and a positive bacterial culture (>10₈ colony forming units).

Characterization of Urine Protein Profiles Associated with Individual Patient Groups

It was necessary to determine the urine protein profile of a ‘normal’ kidney transplant, and this was done by selecting urines from patients with immediate and persistent good graft function that had normal graft histology on protocol biopsy. This stringently defined control group is distinct as it includes histology; other groups attempting similar studies have inferred normal histology from a stable serum creatinine (57,56,97).

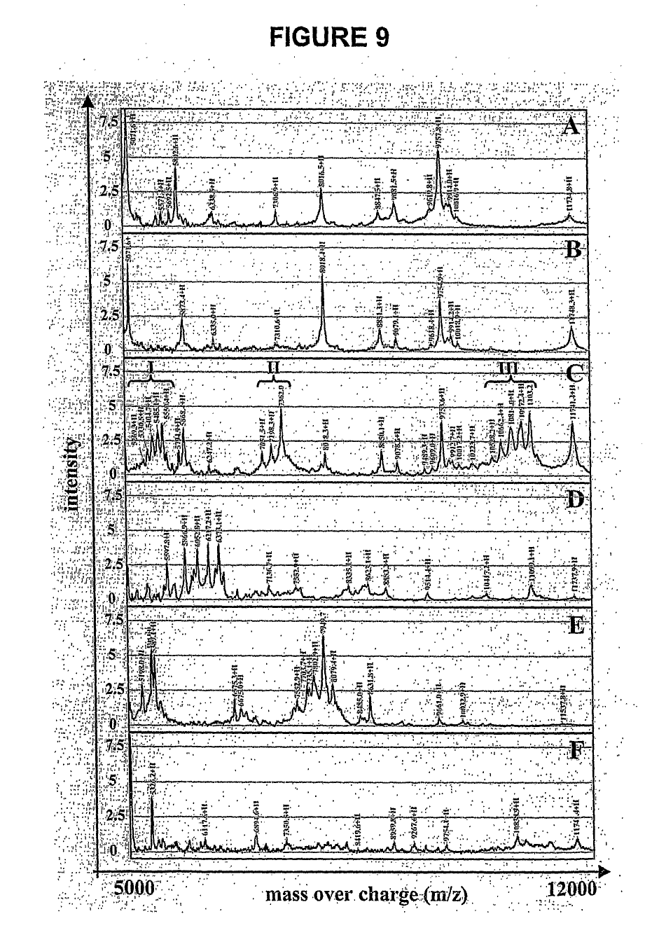

In the m/z range from 5000 to 12000 two distinct urine protein patterns were observed when comparing the normal control group or stable transplant group to the acute clinical rejection group. One urine protein profile (rejection pattern) had prominent peak clusters in three regions corresponding to m/z values of 5270-5550 (Region I; 5 peaks), 7050-7360 (Region II; 3 peaks), and 10530-11100 (Region III; 5 peaks) that always occurred together, whereas the other urine protein profile (normal pattern) had no peak clusters in these m/z regions (FIG. 9). All 28 urine samples (100%) from the normal control group, 18 of 22 urine samples (82%) from the stable transplant group, and 1 of 18 urine samples (6%) from the acute clinical rejection group showed the normal pattern. The rejection pattern was detected significantly more often in the acute clinical rejection group (17 of 18; 94%) than in the stable transplant group (4 of 22; 18%) (p<0.0001) (FIG. 10). The ATN, the recurrent (or de novo) glomerulopathy and the UTI groups had urine protein profiles that were different from both the normal and the rejection pattern (FIG. 9 and FIG. 10).

Adherence to this stringent definition of ‘normal’ demonstrates that the urine protein profile from 18 of 22 patients (82%) in the stable transplant group was similar to the urine profile of normal non-transplanted individuals. The reliable identification of the urine protein pattern of the normal kidney transplant allowed for the clear differentiation, on visual inspection alone, of a distinct urine protein profile in the group with acute rejection (FIG. 10).

The urine protein profile in the ATN and glomerulopathy groups did not show the pattern of rejection. Both ATN and glomerulopathies are important in the differential diagnosis of allograft dysfunction, and may represent pathophysiological models of allograft injury distinct from that due to the alloimmune response. Whereas ATN can be regarded as a model of injury to the tubules due to ischemia-reperfusion, in the glomerulopathies, the injury, although presumably immune in nature, is largely centered on the glomerular capillary. As these two pathological states did not show the characteristic pattern of rejection, the inventors infer that the urine proteins detected in acute rejection are related to recipient immune cells infiltrating the graft and/or to tubular epithelial cells that are involved in the allo-directed inflammation. It is acknowledged, however, that the possibility that the urine proteins associated with rejection may also be found in other causes of tubular-based pathology (i.e. calcineurin-inhibitor-toxicity, polyomavirus type BK-nephropathy, pyelonephritis) cannot be excluded. These latter outcomes are of relatively lower frequency in the patient population of the present study, such that it was not possible to generate pure examples of each in sufficient number to make any reliable conclusions. Indeed, it is notable, that in this patient population (n=212) only one patient (0.5%) developed polyomavirus type BK-nephropathy, which is a much lower incidence than reported from another centre (8%) (10).

### Influence of CMV-Viremia on Urine Protein Profile Pattern

Twenty-seven of 40 patients (68%) in the stable transplant and acute clinical rejection groups were tested for the presence of CMV-viremia at the time of renal allograft biopsy. Five patients tested positive; however none had or developed CMV-disease subsequently. CMV-viremia was found in 2 of 21 patients (10%) with the rejection pattern and in 3 of 19 patients (16%) with the normal pattern (P=0.83) (Table 5). No additional peaks in the urine protein profiles from patients who had CMV-viremia were detected.

An additional potential confounder of the diagnostic specificity of the urine protein profile observed in allograft rejection is systemic inflammation that could lead to the filtration of inflammatory proteins (e.g. chemokines, cytokines) by the transplant kidney. Post-transplant CMV viremia, which has a high incidence in kidney transplant recipients (101,102) but very rarely infects the allograft (103,104), is one of the most common causes of systemic inflammation post-transplant.

Indeed, the inventors have previously reported that CMV-viremia is a significant confounding variable when examining activated T-cells in the circulation as a possible non-invasive correlate of biopsy proven allograft rejection (61). In the current study, no correlation was found between CMV-viremia and the urine profile of rejection, which argues against systemic inflammation associated with CMV viremia as a significant confounding factor. While this does not rule out the possibility that other systemic inflammatory processes may mimic the urine profile seen in allograft rejection, it suggests that this is probably less likely.

### Specificity of Profile-Sequential Urine Protein Profile Analysis

To further determine the specificity of the normal and rejection pattern, serial urine protein profiles in the stable transplant and acute clinical rejection groups were examined and correlated with the clinico-pathological course of the renal allograft. In particular, four specific outcomes were of interest: [1] the stable course persisted; [2] the stable transplant patient subsequently had an acute clinical rejection; [3] acute clinical rejection resolved to a stable course; [4] acute clinical rejection recurred.

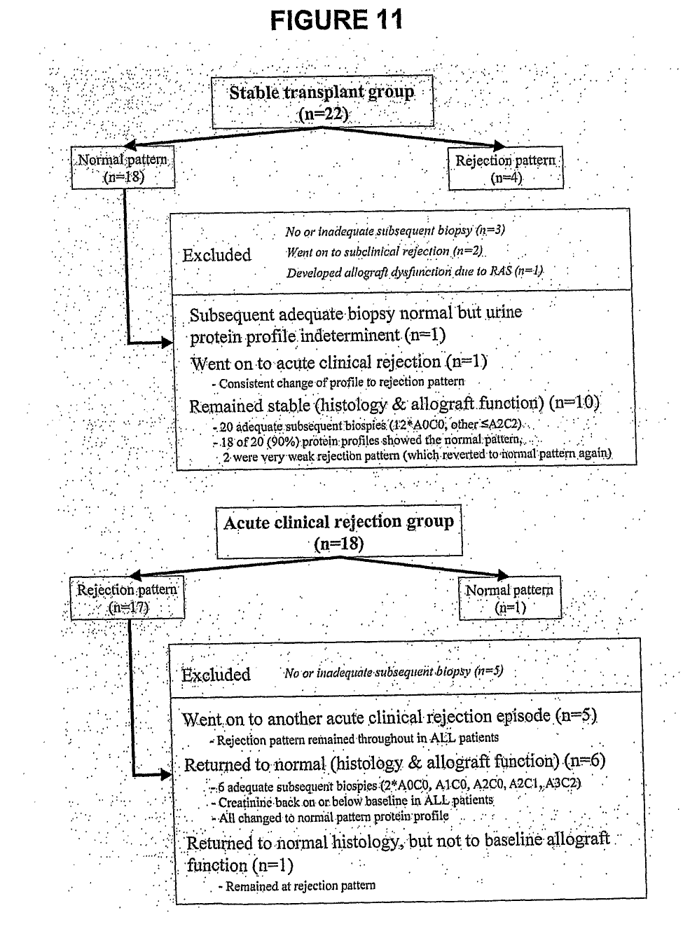

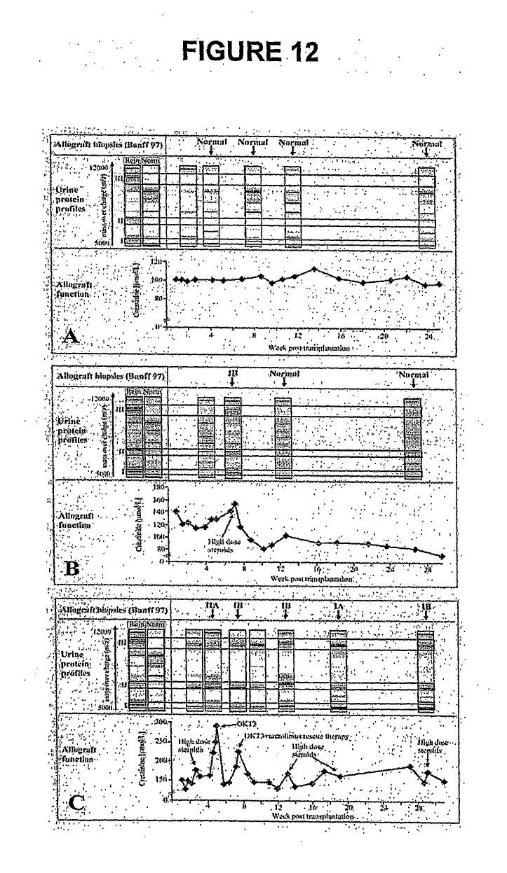

In the stable transplant group, there were sufficient urine and histology samples for sequential analysis to evaluate 12 of the 18 patients that originally had a normal pattern (FIG. 11). One patient went on to have stable allograft function and two normal protocol biopsies, but the urine profile could not be classified. One patient developed acute clinical rejection (Banff type IA) and the urine protein profile changed from the normal to the rejection pattern. In 10 patients stable allograft function persisted and 20 subsequent protocol biopsies were interpreted as normal (n=18) or borderline rejection (n=2). Eight of these 10 patients showed the normal pattern throughout (FIG. 12A), whereas two patients exhibited the rejection pattern in a single urine sample that subsequently reverted to the normal pattern.

In the acute clinical rejection group, there were sufficient urine and histology samples for sequential analysis to evaluate 12 of the 17 patients that originally had a rejection pattern (FIG. 11).

One patient had two subsequent normal protocol biopsies, but the creatinine remained elevated at the level seen during the acute rejection episode (20% above baseline) and the urine always showed the rejection pattern. In 6 patients the allograft function returned to baseline and subsequent protocol biopsies were interpreted as normal (n=3) or borderline rejection (n=3). One patient had acute clinical rejection (Banff IB) on week 7 post-transplant. After treatment with high dose oral steroids the serum creatinine normalized and remained stable. Subsequent allograft biopsies were normal. The urine protein profile showed the normal pattern 3 week prior to the rejection episode, changed to the rejection pattern at the time of rejection, and returned to the normal pattern consistent with the subsequent allograft biopsies and the allograft function. All urine samples from these patients changed to the normal pattern (FIG. 12B). Five patients had further episodes of acute clinical rejections and all of them kept the rejection pattern throughout (FIG. 12C). This was despite treatment with OKT3, high dose steroids and increased baseline immunosuppression.

It was of interest that the protein profile of rejection was similar regardless of the histological severity (Banff IA vs. IB) or type (Banff IA/B vs. IIA). This finding might represent a relative limitation of the technique of urine proteomics in identifying biomarkers specific for tubulo-interstitial versus vascular rejection. However, because the assignment of histological severity/type of acute rejection is based upon a small biopsy sample of a large organ, urine profiling, which is representative of the entire allograft, may be pointing to the extent of heterogeneity of inflammation within the allograft, a fact that renal transplant pathologists are well aware of (49). The correlation between the changes in serial urine profiles and the clinico-pathological course of the patients provided additional support that the detected proteins are related to acute allograft rejection.

### Accuracy

Although there were significant differences in the urine profiles between the stable transplant and the acute clinical rejection groups, there were also one ‘false negative’ and four ‘false positives’ samples. The only patient with the ‘false negative’ urine profile in the acute clinical rejection group had no specific clinical or demographic feature. That patient had a course of a subclinical rejection (ai3t3g0v0) followed by a clinical rejection (ai3t3g0v1)—both treated with oral high dose steroids—and returned to normal histology (ai0t1g0v0) 15 weeks later. The inventors found no obvious explanation for this ‘false negative’ result. Theoretically, a low protein concentration in dilute urine may influence the ability to detect a rejection pattern. However, the protein concentration of the urine samples from the stable transplant and the acute clinical rejection group were similar, making inadequate protein load an unlikely explanation for the absence of the rejection pattern. The four patients with ‘false positive’ urine profiles in the stable transplant group also had no specific clinical or demographic features at the time of the biopsy. However, one of them went on to subclinical rejection (ai1t3g0v0) 9 weeks later and one experienced an acute clinical rejection and polyomavirus type BK-nephropathy (BK-NP) 13 weeks later. The other two patients had a normal transplant course with stable graft function. There are mainly two possible explanations for these ‘false positive’ results. First, they are true ‘false positives’ and cannot be explained. Second, they are not ‘false positives’ as the urine profile may be detecting an early rejection process that was missed by the allograft biopsy (i.e. sampling error) (100,49).

### EXAMPLE 6

### Impact of Intrinsic Factors on the Detection of the Rejection Pattern

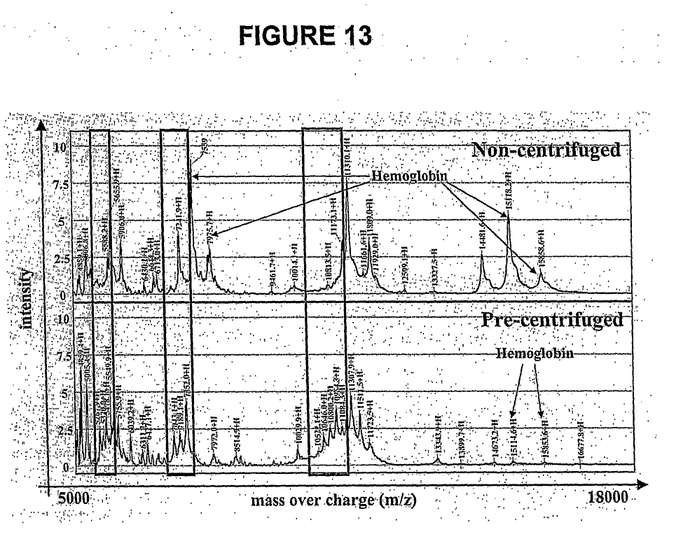

As the urine samples from the transplanted patients were stored non-centrifuged at −80 C, subsequent cell lysis due to freeze-thawing, the analysed urine samples will contain intracellular proteins from cells present in the urine. To investigate whether the release of intracellular proteins of red blood cells (RBC), leucocytes and tubular epithelial cells due to freeze-thawing is responsible for generating the rejection pattern, the inventors compared an ‘acute clinical rejection’ urine sample frozen with and without pre-centrifugation (FIG. 13). In this case, the urine sample was collected from a clinically rejecting patient at day 6 post-transplant. The patient had persistent hematuria since the time of transplantation, which can be easily seen by the peaks consistent with hemoglobin in the non-centrifuged sample. In the pre-centrifuged sample these peaks almost disappeared and the rejection pattern could be clearly seen. Based on this documented case it is presumed that the rejection pattern proteins are not intra-cytoplasmatic proteins that are released after disruption of the cell membrane due to freeze-thawing. In addition, this experiment is a good example of ion suppression due to high abundant proteins (i.e. hemoglobin in this sample).

Urine profiles of the various groups could have been altered by the procedures of urine collection and storage. Due to the fact that all urine samples were stored non-centrifuged, the rejection pattern may have derived from intracellular proteins of leucocytes, RBC or tubular epithelial cells released after a freeze-thaw cycle. Interestingly, in one of the rejection cases the inventors found that lysis of RBC prevented the detection of the rejection pattern due to ion suppression. However, precentrifugation to remove the RBC prior to freeze-thawing of this sample allowed the rejection pattern to be detected. Therefore, this argues that the pattern is not necessarily derived from cell lysis associated with a freeze-thaw cycle.

### EXAMPLE 7

### Identification of Proteins Associated with Acute Renal Allograft Rejection

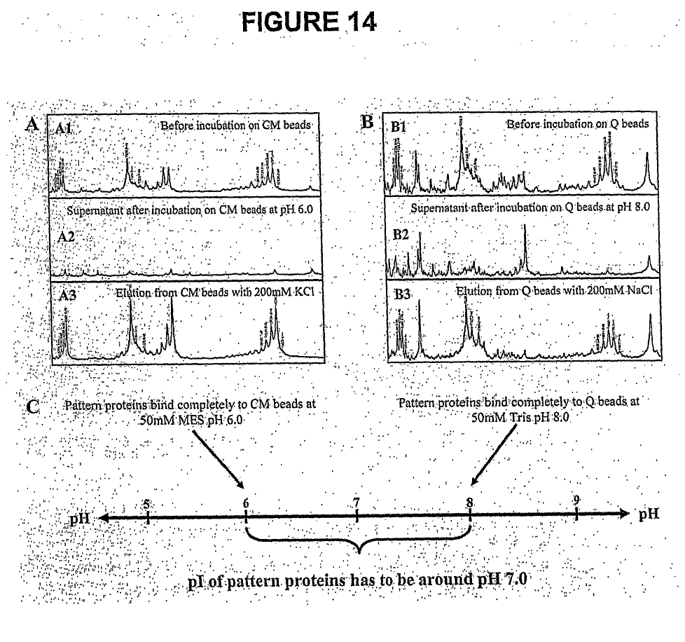

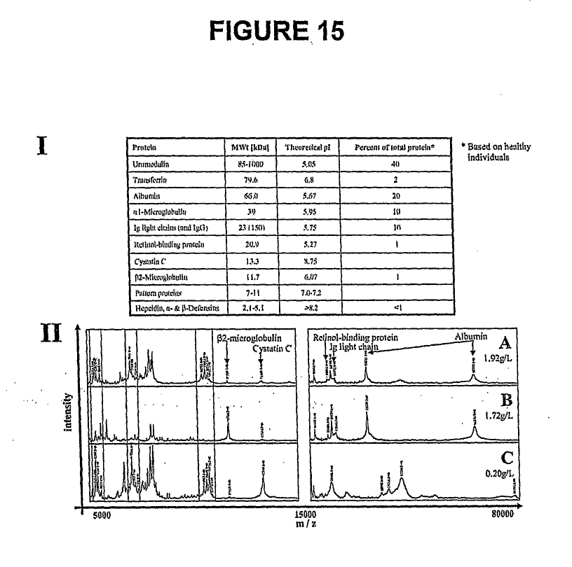

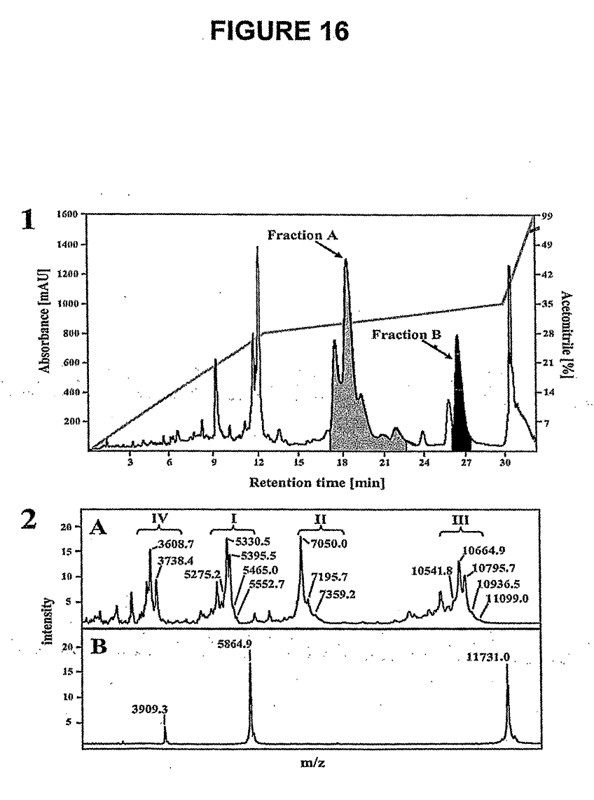

The inventors first determined the pI of the rejection pattern proteins in order to subsequently use an extraction method (i.e. ion-exchange beads) as an initial step to concentrate the target proteins. With the use of cation- and anion-exchange beads the pI was estimated to be around 7.0 (FIG. 14). This allowed binding the target protein to anion-exchange beads at a pH 6.2, and subsequently eluting them with potassium as a counter-ion. As expected from their contribution to the total protein content of urine, the purification on the cation-exchange beads at pH 6.2 resulted in a significant decrease of the total protein concentration in the elution fraction from 1.92 g/L (before incubation on beads, A) to 0.2 g/L (C) (measured with the BCA protein assay, Pierce, Rockford, Ill., USA). This initial step resulted not only in a substantial concentration of the target proteins (100 fold) but also in a decrease of the complexity of the sample. Indeed, many major components of the urine proteome could be separated out, as their pIs are below that of the target proteins (e.g. albumin: pI 5.67; retinal-binding protein: pI 5.27) (FIG. 15). Additional and final purification was achieved by RP-HPLC, where the target proteins could be almost perfectly separated (FIG. 16).

### Protein Profile Fragments

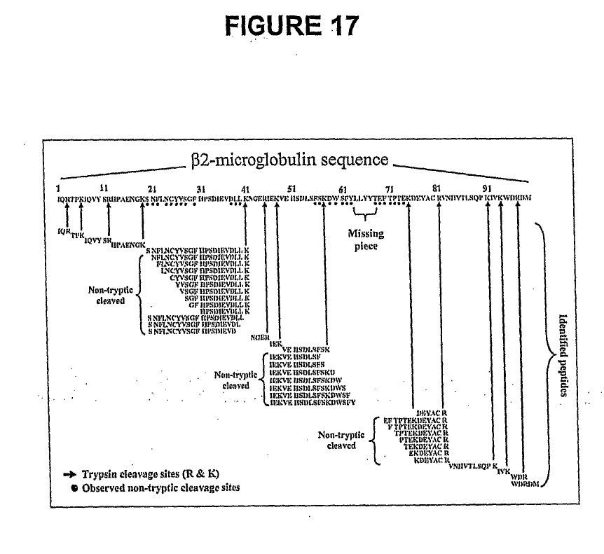

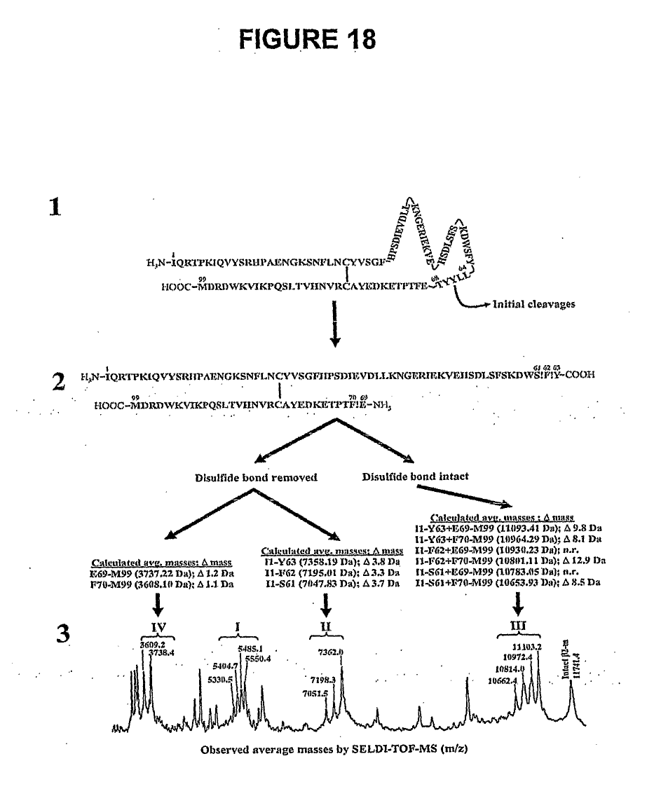

After in-solution digestion, the target proteins were identified by μLCMS and liquid chromatography coupled to tandem mass spectrometry (μLC-MS/MS) as a cleaved form of β2-microglobulin. As all the protein(s) responsible for the peak clusters remained in one single fraction after two purification steps involving cation-exchange and reverse-phase chromatography, a close relationship between each of the peaks seemed obvious. In fact, analysis of the purified and trypsin-digested sample by μLC-MALDI-MS (/MS) revealed that all peptides found belong to one protein, namely β2-microglobulin. Complete decoupling of μLC and MALDI-MS (/MS) techniques enables detailed analysis of the deposited sample without any time constraints. This feature was used to find small and/or low-abundant peptides by single MS mass measurement and confirming their sequence by MS/MS, resulting in almost complete coverage of the 99 amino acid long β2-microglobulin sequence, with the exception of a five amino acid long peptide (L64-T68) (FIG. 17, Table 6 as supplemental information (SEQ ID NOS:11-49)). In addition, 26 non-tryptic cleaved peptides of the β2-microglobulin-sequence could be identified. Based on these and the missing piece (L64-T68) one can explain all the characteristic protein peak clusters detected by SELDI-TOF-MS with cleaved forms of β2-microglobulin (FIG. 18). Based on the protein fragments detected by SELDI-TOF-MS with the highest intensities, the main non-tryptic cleavage sites may be S61, T68 and E69, resulting in two protein fragments with a predicted molecular weight of 10653.93 Da and 10783.05 Da, respectively. The observed masses by SELDI-TOF-MS are 10650.7 Da (−3.2 Da) and 10782.1 Da (−1 Da). By theoretically removing the disulphide bond (C25-C80) from the S61-, T68- and E69-cleaved β2-microglobulin, three peptides will result:

[I]I1QRTPKIQVYSRHPAENGKSNFLNCYVSGFHPSDIEVDLLKNGERI EKVEHSDLSFSKDWS61 (SEQ ID NO:4) with a predicted molecular weight of 7047.83 Da (SELDI-TOF-MS mass 7042.9 Da (−4.9 Da));

[II] E69FTPTEKDEYACRVNHVTLSQPKIVKWDRDM99 (SEQ ID NO: 5) with a predicted molecular weight of 3737.22 Da (SELDI-TOF-MS mass 3733.0 Da (−4.2 Da)); and

[III] F70TPTEKDEYACRVNHVTLSQPKIVKWDRDM99 (SEQ ID NO:6) with a predicted molecular weight of 3608.10 Da (SELDI-TOF-MS mass 3603.6 Da (−4.5 Da))

Therefore, these three cleavage sites combined with or without the theoretical removal of the disulphide bond explain five of the seven major peaks detected by SELDI-TOF-MS (FIG. 18). The remaining two peaks at 5322.6 Da and 5387.2 Da are consistent with the double charged ions of the 10650.7 Da and 10782.1 Da protein fragments. This explanation is further supported by the disappearance of the SELDI-TOF-MS peaks at 5322.6 Da, 5387.2 Da, 10650.7 Da and 10782.1 Da after reduction and alkylation, while maintaining the peaks at 7042.9 Da, 3733.0 Da and 3603.6 Da (with an additional ˜65 Da due to reaction with iodoacteamide) (data not shown).

However, the initial described rejection pattern had prominent peak clusters at 5.27-5.55 kDa (5 peaks), 7.05-7.36 kDa (3 peaks), and 10.53-11.1 kDa (5 peaks). The unaccounted SELDI-TOF-MS peaks at 7.2 kD and 7.36 kD, as well as the concomitant appearing or disappearing peaks at 10.95 kD and 11.1 kD (double charged ions at 5.48 kDa and 5.55 kDa) can be explained by a different initial cleavage site (Y63 instead of S61) with subsequent partial removing of Y63 and F62 (FIG. 18).

The two last remaining unaccounted peaks of the original rejection pattern (5.27 kDa and 10.53 kDa) can most likely be explained by removing F70, which was an observed cleavage site (FIG. 17).

Proteins can be separated based on [I] their molecular weight, [II] their pI and [III] their, hydrophobicity. The use of ion-exchange beads as a first step to purify the target proteins with a pI of 7.0 from urine offered two advantages. First, it allows one to concentrate the target proteins, and second, many proteins with lower pIs could be excluded. Subsequently, the high-resolution ability of RP-HPLC allowed purifying the cleaved β2-microglobulin. Indeed, it was even possible to separate the cleaved form (eluted at around 31% acetonitrile) from the intact form (eluted at around 33% acetonitrile), which only differ by seven amino acids. Identification of cleaved β2-microglobulin by μLC-MS and μLC-MS/MS is very reliable. Not only were all the peptides corresponding to the β2-microglobulin sequence (without the cleaved piece ‘F62YLLYYT68’ (SEQ ID NO:10)) found and confirmed by MS/MS, but the observed and predicted cleaved forms could explain 11 of 13 peaks of the rejection pattern detected by SELDI-TOF-MS. However, the question remains, why cleaved β2-microglobulin produces the observed multiple peaks on the SELDI-TOF-MS spectra. β2-microglobulin consists of 99 amino acids and contains one disulphide bond (C25-C80). Purified human β2-microglobulin from urine is not fragmented when analysed by SELDI-TOF-MS and only the double charged species is observed beside the parent ion (FIGS. 16, 2). However, after cleavage of the above-mentioned piece two chains result, which are connected by the disulphide bond (C25-C80). During the ionization process the disulphide bond may break (105,106) in some cleaved β2-microglobulin molecules resulting in additional detection of the two single chains.

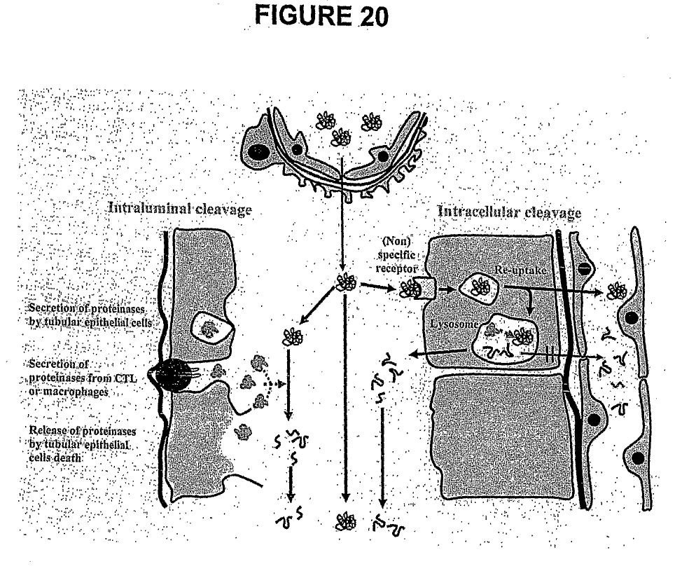

Based on the SELDI-TOF-MS detected β2-microglobulin fragments, the initial non-tryptic cleavage sites were postulated to be Y63 and T68. Thereafter additional major cleavages occur at S61, F62 and E69, resulting in 11 of 13 peaks contributing to the rejection pattern (FIG. 18). However, more non-tryptic cleavage sites were found (F22, N24, C25, F70, T71, P72, T73 and E74) which could not be assigned to corresponding SELDI-TOF-MS peaks. The proteinase(s) involved in the initial cleavage as well as the proteinase(s) responsible for further fragmentation of β2-microglobulin are not determined yet. Cleavage of β2-microglobulin could either have happened intracellularly (i.e. lysosomal enzymes) or intraluminally (i.e. secreted proteinases from CTL, macrophages or tubular epithelial cells; release of lysosomal enzymes due to tubular epithelial cell death) (FIG. 20).

β2-microglobulin is freely filtered through the glomerular barrier and is normally reabsorbed by proximal tubular epithelial cells to a large extent. Therefore, changes in β2-microglobulin metabolism and excretion are mainly dependent on the function of the tubular epithelial cells. In addition, proteinases in urine may mostly be derived from these cells (107,108,109). Taken together, the presence of cleaved β2-microglobulin in urine is most likely to be associated with tubular epithelial cell stress/injury. Interestingly, in patients with pure humoral rejection (n=3, data not shown), which does not target the tubular cells, cleaved β2-microglobulin was not detectable by SELDI-TOF-MS further supporting the association between tubular cell stress/injury and the presence of cleaved β2-microglobulin. Whether cleaved β2-microglobulin is specific for tubular cell stress/injury due to rejection is not known yet and needs to be addressed in further analysis of samples with different pathologies affecting the tubuli (i.e. CNI-toxicity, polyomavirus type BK-nephropathy, pyelonephritis).

Alternatively to the hypothesis that urinary cleaved β2-microglobulin is derived from filtration of intact recipient β2-microglobulin with subsequent intracellular or intraluminal fragmentation in the allograft, it could also be derived directly from kidney donor allograft cells (e.g. tubular epithelial cells) or from recipient immune system cells in the allograft (e.g. CTL, macrophages).

### EXAMPLE 8

Other approaches can be used to profile different subsets of urine proteins for their potential as biomarkers for renal allograft rejection. Such approaches include comparative analysis of urine samples from stable transplants and patients undergoing rejection (i.e. differential protein profiling).

The Aebersold laboratory introduced the concept of isotope coded affinity tags (ICAT), in an effort to provide a means for direct comparison of protein levels in two samples by mass spectrometry (70). The ICAT reagent is in two structurally identical forms, which only differ by the presence of heavy, H, or light, L, species of stable isotopes of carbon or hydrogen (i.e. C_{12/}C₁₃ or H₁/H₂) resulting in H and L forms of ICAT. Equal amounts of the two samples to be compared are reduced, labeled separately, each with only one species of ICAT, and then pooled for processing. ICAT reacts with free —SH groups and introduces a selectable biotin affinity tag which allows for the isolation of the tag labelled peptides from the overall digest. This step was designed to reduce the overall complexity of the samples in the subsequent chromatographic and mass spectrometric steps. The tagged peptides can be separated by 1 or 2 dimensional μLC on-line with a mass spectrometer. In single MS mode peptides from the same protein species but labelled with the heavy and light forms of the tag will display a predictable separation in m/z depending on the charge state of the peptides. Integration of the areas of the isotope cluster for the H and L species provides a basis for comparing their relative abundance.

Subsequent analysis of the parent ion by tandem MS provides protein identification. Thus in a single experiment it is possible to obtain information on relative protein abundance and identity of the altered expression patterns. The limitations of the approach relate to the relatively narrow dynamic range (i.e. ˜10 fold) and the requirement for cysteines in the proteins.

Another approach for differential protein profiling employs digestion in the presence of O₁₆ or O₁₈ (110). Equal quantities of urinary proteins from the groups to be compared will be dialysed and lyophilised. The samples will then be resuspended in buffer containing exclusively either O₁₆ or O₁₈ H₂O. During trypsin digestion peptide bonds C terminal to the basic residues are hydrolysed resulting in the incorporation of OH into the C terminal carboxyl group. Thus by digesting the two protein mixtures to be compared in different forms of H₂O sets of peptides differing by 2 mass units are generated. Combining equal quantities of the protein digests and fractionating in a similar fashion to the ICAT provides a means of performing a similar type of quantitative comparison and protein identification. This scheme labels all peptides and is only dependent on the presence of cleavage sites for trypsin rather than cysteine.

In summary, the results from the ICAT and the hydrolytic labelling offer the means to obtain broad comparative analysis of the urine samples of interest. However, both methods do not allow for high throughput analysis making the selection of few clinically well defined samples mandatory to allow meaningful interpretation.

### EXAMPLE 9

### Production of β2-Microglobulin Fragments

### First Step: Initial Cleavage of Intact Urinary β2-Microglobulin

### [i] pH Dependence of Initial Cleavage of Intact Urinary β2-Microglobulin

A urine from a patient, which showed in the SELDI-TOF-MS spectrum both the intact and the cleaved form of β2-microglobulin, was brought to pH 3, 4.5, 6 and 8. After 6 to 24 hours the intact form of β2-microglobulin was not detectable anymore in urines with pH 3 and 4.5, whereas it was detectable in unchanged intensity in urines with pH 6 and 8.

[ii] Inhibition of Initial Cleavage of Intact Urinary β2-Microglobulin by Pepstatin

Pepstatin is a well-established inhibitor of aspartic proteinases. A urine from a patient, which showed in the SELDI-TOF-MS spectrum both the intact and the cleaved form of β2-microglobulin, was brought to pH 4.5 that the inventors demonstrated in [i] to be required for initial cleavage of intact urinary β2-microglobulin. Different proteinase-inhibitors were added (Pepstatin and Complete Mini EDTA-free, both from Roche, Switzerland). After 6 to 24 hours the intact form of β2-microglobulin was not detectable anymore in urine spiked with Complete Mini EDTA-free and in untreated urine, whereas it was still detectable in unchanged intensity in urines spikes with Pepstatin.

The initial cleavage of intact urinary β2-microglobulin is dependent on a pH<6 and can be inhibited by Pepstatin. This indicates that the initial cleavage is done by aspartic proteinase(s), which are known to be mainly active at lower pH. So far, two aspartic proteinases were found in renal tubular epithelial cells and in urine (i.e. cathepsin D and napsin A), and one or both may be responsible for the initial cleavage of intact urinary β2-microglobulin. Based on the preliminary data it appears that the initial cleavage sites are at Y63 and L65 (see FIG. 18).

### Second Step: Major Cleavages on Long Chain (I1-Y63) and Short Chain (Y66-M99) of Cleaved β2-Microglobulin

Cleavage of β2-microglobulin by aspartic proteinase(s) creates two chains that are still connected through the disulphide bond (C25-C80). The long chain of cleaved β2-microglobulin consists of 63 amino acids (I1-Y63), the short chain of 34 amino acids (Y66-M99).

### [i] Major Cleavages on Long Chain (I1-Y63)

Further confirmed non-tryptic cleavage sites on the long chain occur at F62 and then at S61, resulting in three major forms of long chains with calculated molecular weights of 7358.19 Da (I1-Y63), 7195.01 Da (I1-F62) and 7047.83 Da (I1-S61) (see FIG. 25B). The cleavage of the two amino acids Y63 and F62 occur even at pH 6 and can be inhibited by Complete Mini EDTA-free (inhibits serine and cysteine proteinases), but not by 20 mmol/L EDTA (inhibits metalloproteinases) or Pepstatin. This suggests that the cleavage of these two amino acids is performed by serine and/or cysteine proteinases. In addition, three cleavage sites on the long chain have been identified by μLC-MS/MS (F22, N24 and C25), however, the resulting fragments were not detectable by SELDI-TOF-MS.

### [i] Major Cleavages on Short Chain (Y66-M99)

Many cleavage sites on the short chain have been confirmed by μLC-MS/MS (K75, E74, T73, P72, T71, F70 and E69). Only two of the resulting forms can be confidently found by SELDI-TOF-MS with calculated molecular weights of 3737.22 Da (E69-M99) and 3608.10 Da (F70-M99) (see FIG. 18). In addition, SELDI-TOF-MS rarely detected proteins with molecular masses that are consistent with short chain forms of Y66-M99, Y67-M99 and T68-M99. This further supports one of the proposed initial cleavage sites at L65. Preliminary data suggest that both aspartic and serine/cysteine proteinase(s) are involved in cleavages on the short chain of cleaved β2-microglobulin.

During analysis by SELDI-TOF-MS disulphide bonds can break resulting in the detection of the different forms of single short and long chains of cleaved β2-microglobulin. However, cleaved β2-microglobulin forms consisting of both chains, which are connected through the disulphide bond, are still present (see FIG. 18).

### EXAMPLE 10

### Determination of the Protease Family Responsible for β2-Microglobulin Cleavage

One mL of a urine sample from a healthy individual (total protein 110 mg/L, creatinine 18.9 mM, pH 5) and 1 mL of a urine sample from a patient with an acute clinical rejection episode showing the protein peak clusters (total protein 230 mg/L, creatinine 11.6 mM, pH 5) were incubated for 16 hours at 37° C. to degrade existing intact and cleaved β2-m. Four hundred μL of each sample were mixed with sodium acetate pH 5 (final concentration 166 mM) to ensure, stable pH and divided into four portions of 100 μL. Pepstatin (final concentration 14.5 μM), Complete-Mini (EDTA-free) (final concentration 0.2 tablets/mL), EDTA (final concentration 20 mM) or no protease inhibitors were added to each portion. Another 100 μL of each urine sample was brought to pH 6 by adding MES pH 6 (final concentration 166 mM). Purified intact β2-microglobulin (final concentration 10 mg/L) was added to all portions. SELDI-TOF-MS analysis was performed immediately and after 1, 2, 4, 6 and 24 hours of incubation at 37° C.

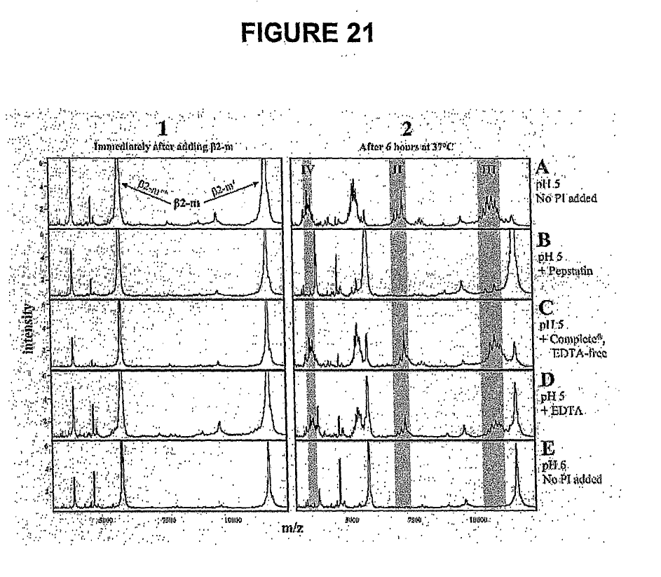

The characteristic protein peak clusters of cleaved β2-microglobulin could be generated by spiking intact β2-microglobulin into even normal urine under specific conditions. Cleavage of intact β2-microglobulin was only observed at a urine pH<6 and could be inhibited by the aspartic protease inhibitor pepstatin, but not by cysteine & serine protease inhibitors (Complete-Mini (EDTA-free)) or a metalloprotease inhibitor (EDTA) (FIG. 21).

### EXAMPLE 11

### Estimation, of Protease Amount in Different Samples

One hundred μL of a urine sample from a healthy individual (important parameters stated above) and 100 μL of a urine sample from a patient with the protein peak clusters (important parameters stated above) were incubated for 16 hours at 37° C. to degrade existing intact and cleaved β2-microglobulin. Sodium acetate pH 5 (final concentration 166 mM) was added to ensure stable pH, and then purified intact β2-microglobulin (final concentration 10 mg/L) was spiked into each sample. SELDI-TOF-MS analysis was performed immediately and after 1, 2, 4, 6 and 24 hours of incubation at 37° C.

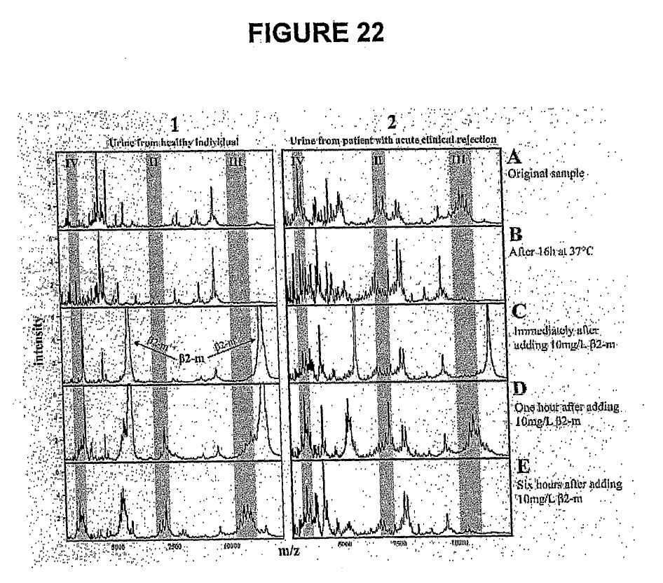

The generated cleaved β2-microglobulin forms were identical in urine samples from a healthy individual and a patient with an acute clinical rejection episode. However, the course of cleavage/degradation was much faster in the latter one, suggesting that more protease activity was present in the urine sample collected during acute clinical allograft rejection (FIG. 22).

Cleaved β2-microglobulin forms representing the protein peak clusters are produced early in the degradation process of β2-microglobulin and are by far the most abundant cleaved β2-microglobulin forms based on peak intensity of the SELDI-TOF-MS spectra. Thereafter more cleavages occur (see FIG. 17) and β2-microglobulin is degraded into many smaller fragments, which were not detectable anymore by SELDI-TOF-MS. This suggests that measurement of cleaved β2-microglobulin forms representing the protein peak clusters should still accurately reflect the amount of previously intact β2-microglobulin they were generated from. As demonstrated in vitro, 10 mg/L β2-microglobulin is completely degraded within 6 hours in a rejection urine sample and within 24 hours in a normal urine sample, respectively (FIG. 22). However, this is unlikely to happen in vivo, as long as intact β2-microglobulin is continuously replenished from serum through glomerular filtration or there is not an excessive amount of aspartic proteases in the sample.

### EXAMPLE 12

### Estimation of Detection Threshold of SELDI-TOF-MS for Cleaved β2-Microglobulin

One mL of a urine sample from a healthy individual (important parameters stated above) was incubated for 16 hours at 37° C. to degrade existing intact and cleaved β2-microglobulin. Sodium acetate pH 5 (final concentration 166 mM) was added to ensure stable pH and the sample was divided into nine portions of 100 μL each. Different amounts of purified intact β2-microglobulin (final concentrations from 0.05-10 mg/L) were spiked into these 100 μL portions. SELDI-TOF-MS analysis was performed immediately and after 6 hours of incubation at 37° C.

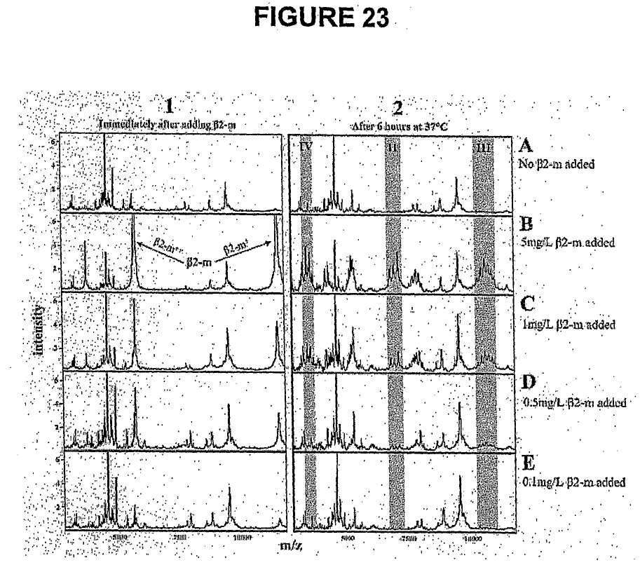

To determine the detection threshold of SELDI-TOF-MS for cleaved β2-microglobulin, we added different amounts of intact β2-microglobulin into a urine from a healthy individual and analysed the samples for the presence of cleaved β2-microglobulin forms after 6 hours of incubation at 37° C. The detection threshold of SELDI-TOF-MS for the cleaved β2-microglobulin was between 0.1 and 0.5 mg/L of added intact β2-microglobulin (FIG. 23), which is at the upper range of normal values of intact urinary β2-microglobulin measured by radioimmunoassay (<0.2 mg/L) as reported by other investigators (13,14,15).

### Effect of pH on β2-Microglobulin Cleavage

As stated above, urine pH is critical for generation of cleaved β2-microglobulin. Therefore, the inventors retrospectively analysed urine pH in all available samples (n=63) from the previous examples where the identified cleaved β2-microglobulin forms were closely associated with acute clinical allograft rejection (10). Urine pH in the acute clinical rejection group (n=18; pH=5.26±0.33, range 4.7-5.8) was significantly lower than in the stable transplant group (n=22; pH=5.58±0.50, range 5.0-6.5) (p=0.037) and the healthy control group (n=23; pH=5.89±0.56, range 5.0-7.0) (p=0.0004). All urine samples with cleaved β2-microglobulin forms detectable by SELDI-TOF-MS had a urine pH<6. However, 20 of the analysed 63 urine samples (1 in the acute clinical rejection group, 11 in the stable transplant group, and 8 in the healthy individuals group) had pH<6 without SELDI-TOF-MS-detectable cleaved β2-microglobulin forms. Based on the experiment presented above (FIG. 23) this can most likely be explained by low initial amounts of intact and subsequently cleaved β2-microglobulin in these samples, which were below the SELDI-TOF-MS detection threshold.

### EXAMPLE 13

### Pathogenesis of Cleaved Urinary β2-Microglobulin

Cleaved urinary β2-microglobulin can be regarded as a marker for tubular cell stress/injury, because all patients in the acute clinical allograft rejection group had at least mild tubulitis (i.e. Banff acute Score ≧i2t2). As was demonstrated, tubular cell stress/injury during allograft rejection can lead to (i) decreased reabsorption of intact β2-microglobulin, (ii) increased amounts of proteases in urine, and (iii) lower urine pH (FIG. 24). Under such conditions substantial amounts of cleaved. β2-microglobulin are generated, which may account for the major part of total urinary β2-microglobulin (=intact and cleaved β2-microglobulin).

Therefore, cleaved urinary β2-microglobulin represents several pathophysiological processes occurring during tubular cell stress/injury related to tubulointerstitial allograft rejection. However, it is not believed that cleaved urinary β2-microglobulin is specific for tubulointerstitial allograft rejection, but may be a sensitive marker for any kind of tubular cell stress/injury (e.g. CI-nephrotoxicity, polyomavirus type BK-nephropathy).

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Hariharan S, Johnson C P, Bresnahan B A, Taranto S E, McIntosh M J, Stablein D: Improved graft survival after renal transplantation in the United States, 1988 to 1996. N Engl J Med 342:605-612, 2000
2. Meier-Kriesche H U, Schold J D, Srinivas T R, Kaplan B: Lack of improvement in renal allograft survival despite a marked decrease in acute rejection rates over the most recent era. Am J Transplant 4:378-383, 2004
3. Pascual M, Theruvath T, Kawai T, Tolkoff-Rubin N, Cosimi A B: Strategies to improve longterm outcomes after renal transplantation. N Engl J Med 346:580-590, 2002
4. Meier-Kriesche H U, Ojo A O, Hanson J A, Cibrik D M, Punch J D, Leichtman A B, Kaplan B: Increased impact of acute rejection on chronic allograft failure in recent era. Transplantation 70:1098-1100, 2000
5. Almond P S, Matas A, Gillingham K, Dunn D L, Payne W D, Gores P, Gruessner R, Najarian J S: Risk factors for chronic rejection in renal allograft recipients. Transplantation 55:752-756, 1993
6. Gourishankar S, Hunsicker L G, Jhangri G S, Cockfield S M, Halloran P F: The stability of the glomerular filtration rate after renal transplantation is improving. J Am Soc Nephrol 14:2387-2394, 2003
7. Hariharan S, McBride M A, Cohen E P: Evolution of endpoints for renal transplant outcome. Am J Transplant 3:933-941, 2003
8. Kreis H A and Ponticelli C: Causes of late renal allograft loss: chronic allograft dysfunction, death, and other factors. Transplantation 71:SS5-SS9, 2001
9. Kasiske B L, Snyder J J, Gilbertson D T, Wang C: Cancer after kidney transplantation in the United States. Am J Transplant 4:905-913, 2004
10. Hirsch H H, Knowles W, Dickenmann M, Passweg J, Klimkait T, Mihatsch M J, Steiger J: Prospective study of polyomavirus type BK replication and nephropathy in renal-transplant recipients. N Engl J Med 347:488-496, 2002
11. Mylonakis E, Goes N, Rubin R H, Cosimi A B, Colvin R B, Fishman J A: BK virus in solid organ transplant recipients: an emerging syndrome. Transplantation 72:1587-1592, 2001
12. Kirk A D: Less is more; maintenance minimization as a step toward tolerance. Am J Transplant 3:643-645, 2003
13. Le Moine A, Goldman M, Abramowicz D: Multiple pathways to allograft rejection. Transplantation 73:1373-1381, 2002
14. Baldwin W M, III, Larsen C P, Fairchild R L: Innate immune responses to transplants: a significant variable with cadaver donors. Immunity 14:369-376, 2001
15. Pratschke J, Wilhelm M J, Kusaka M, Beato F, Milford E L, Hancock W W, Tilney N L: Accelerated rejection of renal allografts from brain-dead donors. Ann Surg 232:263-271, 2000
16. Paul W E and Seder R A: Lymphocyte responses and cytokines. Cell 76:241-251, 1994
17. Sayegh M H, Akalin E, Hancock W W, Russell M E, Carpenter C B, Linsley P S, Turka L A: CD28-B7 blockade after alloantigenic challenge in vivo inhibits Th1 cytokines but spares Th2. J Exp Med 18.1:1869-1874, 1995
18. Simeonovic C J, Townsend M J, Karupiah G, Wilson J D, Zarb J C, Mann D A, Young I G: Analysis of the Th1/Th2 paradigm in transplantation: interferon-gamma deficiency converts Th1-type proislet allograft rejection to a Th2-type xenograft-like response. Cell Transplant 8:365-373, 1999
19. Chan S Y, DeBruyne L A, Goodman R E, Eichwald E J, Bishop D K: In vivo depletion of CD8+ T cells results in Th2 cytokine production and alternate mechanisms of allograft rejection. Transplantation 59:1155-1161, 1995
20. Le Moine A and Goldman M: Non-classical pathways of cell-mediated allograft rejection: new challenges for tolerance induction? Am J Transplant 3:101-106, 2003
21. Hongwei W, Nanra R S, Stein A, Avis L, Price A, Hibberd A D: Eosinophils in acute renal allograft rejection. Transpl Immunol 2:41-46, 1994
22. Kormendi F and Amend W J, Jr.: The importance of eosinophil cells in kidney allograft rejection. Transplantation 45:537-539, 1988
23. Nolan C R, Saenz K P, Thomas C A, III, Murphy K D: Role of the eosinophil in chronic vascular rejection of renal allografts. Am J Kidney Dis 26:634-642, 1995
24. Poulin L F, Richard M, Le Moine A, Kiss R, McKenzie A N, Goldman M, Renauld J C, Van Snick J, Braun M Y: Interleukin-9 promotes eosinophilic rejection of mouse heart allografts. Transplantation 76:572-577, 2003
25. Ten R M, Gleich G J, Holley K E, Perkins J D, Torres V E: Eosinophil granule major basic protein in acute renal allograft rejection. Transplantation 47:959-963, 1989
26. Lakkis F G, Arakelov A, Konieczny B T, Inoue Y: Immunologic ‘ignorance’ of vascularized organ transplants in the absence of secondary lymphoid tissue. Nat Med 6:686-688, 2000
27. Racusen L C, Colvin R B, Solez K, Mihatsch M J, Halloran P F, Campbell P M, Cecka M J, Cosyns J P, Demetris A J, Fishbein M C, Fogo A, Furness P, Gibson I W, Glotz D, Hayry P, Hunsickern L, Kashgarian M, Kerman R, Magil A J, Montgomery R, Morozumi K, Nickeleit V, Randhawa P, Regele H, Seron D, Seshan S, Sund S, Trpkov K: Antibody-mediated rejection criteria—an addition to the Banff 97 classification of renal allograft rejection. Am J Transplant 3:708-714, 2003
28. Saisu K, Morozumi K, Suzuki K, Fujita K: Significance of interstitial lesions as the early indicator for acute vascular rejection in human renal allografts. Clin Transplant 13 Suppl 1:17-23, 1999
29. Weir M R, Hall-Craggs M, Shen S Y, Posner J N, Alongi S V, Dagher F J, Sadler J H: The prognostic value of the eosinophil in acute renal allograft rejection. Transplantation 41:709-712, 1986
30. Racusen L C, Solez K, Colvin R B, Bonsib S M, Castro M C, Cavallo T, Croker B P, Demetris A J, Drachenberg C B, Fogo A B, Furness P, Gaber L W, Gibson I W, Glotz D, Goldberg J C, Grande J, Halloran P F, Hansen H E, Hartley B, Hayry P J, Hill C M, Hoffman E O, Hunsicker L G, Lindblad A S, Yamaguchi Y,: The Banff 97 working classification of renal allograft pathology. Kidney Int 55:713-723, 1999
31. Macdonald F I, Ashraf S, Picton M, Dyer P A, Parrott N R, Short C D, Roberts I S: Banff criteria as predictors of outcome following acute renal allograft rejection. Nephrol Dial Transplant 14:1692-1697, 1999
32. Feucht H E: Complement C4d in graft capillaries—the missing link in the recognition of humoral alloreactivity. Am J Transplant 3:646-652, 2003
33. Halloran P F: The clinical importance of alloantibody-mediated rejection. Am J Transplant 3:639-640, 2003
34. Nickeleit V, Zeiler M, Gudat F, Thiel G, Mihatsch M J: Detection of the complement degradation product C4d in renal allografts: diagnostic and therapeutic implications. J Am Soc Nephrol 13:242-251, 2002
35. Regele H, Bohmig G A, Habicht A, Gollowitzer D, Schillinger M, Rockenschaub S, Watschinger B, Kerjaschki D, Exner M: Capillary deposition of complement split product C4d in renal allografts is associated with basement membrane injury in peritubular and glomerular capillaries: a contribution of humoral immunity to chronic allograft rejection. J Am Soc Nephrol 13:2371-2380, 2002
36. Meehan S M, Domer P, Josephson M, Donoghue M, Sadhu A, Ho L T, Aronson A J, Thistlethwaite J R, Haas M: The clinical and pathologic implications of plasmacytic infiltrates in percutaneous renal allograft biopsies. Hum Pathol 32:205-215, 2001
37. Charney D A, Nadasdy T, Lo A W, Racusen L C: Plasma cell-rich acute renal allograft rejection. Transplantation 68:791-797, 1999
38. Sarwal M, Chua M S, Kambham N, Hsieh S C, Satterwhite T, Masek M, Salvatierra O, Jr.: Molecular heterogeneity in acute renal allograft rejection identified by DNA microarray profiling. N Engl J Med 349:125-138, 2003
39. Robertson H, Ali S, McDonnell B J, Burt A D, Kirby J A: Chronic renal allograft dysfunction: the role of T cell-mediated tubular epithelial to mesenchymal cell transition. J Am Soc Nephrol 15:390-397, 2004
40. Libby P and Pober J S: Chronic rejection. Immunity 14:387-397, 2001
41. Halloran P F: Call for revolution: a new approach to describing allograft deterioration. Am J Transplant 2:195-200, 2002
42. Nankivell B J, Borrows R J, Fung C L, O'Connell P J, Allen R D, Chapman J R: The natural history of chronic allograft nephropathy. N Engl J Med 349:2326-2333, 2003
43. Rush D, Nickerson P, Gough J, McKenna R, Grimm P, Cheang M, Trpkov K, Solez K, Jeffery J: Beneficial effects of treatment of early subclinical rejection: a randomized study. J Am Soc Nephrol 9:2129-2134, 1998
44. Rush D N, Henry S F, Jeffery J R, Schroeder T J, Gough J: Histological findings in early routine biopsies of stable renal allograft recipients. Transplantation 57:208-211, 1994
45. Shapiro R, Randhawa P, Jordan M L, Scantlebury V P, Vivas C, Jain A, Corry R J, McCauley J, Johnston J, Donaldson J, Gray E A, Dvorchik I, Hakala T R, Fung J J, Starzl T E: An analysis of early renal transplant protocol biopsies—the high incidence of subclinical tubulitis. Am J Transplant 1:47-50, 2001
46. Gloor J M, Cohen A J, Lager D J, Grande J P, Fidler M E, Velosa J A, Larson T S, Schwab T R, Griffin M D, Prieto M, Nyberg S L, Sterioff S, Kremers W K, Stegall M D: Subclinical rejection in tacrolimus-treated renal transplant recipients. Transplantation 73:1965-1968, 2002
47. Grimm P C, Nickerson P, Gough J, McKenna R, Stern E, Jeffery J, Rush D N: Computerized image analysis of sirius red-stained renal allograft biopsies as a surrogate marker to predict long-term allograft function. J Am Soc Nephrol 14:1662-1668, 2003
48. Nickerson P, Jeffery J, Gough J, McKenna R, Grimm P, Cheang M, Rush D: Identification of clinical and histopathologic risk factors for diminished renal function 2 years posttransplant. J Am Soc Nephrol 9:482-487, 1998
49. Nicholson M L, Wheatley T J, Doughman T M, White S A, Morgan J D, Veitch P S, Furness P N: A prospective randomized trial of three different sizes of core-cutting needle for renal transplant biopsy. Kidney Int 58:390-395, 2000
50. Hernandez-Fuentes M P, Warrens A N, Lechler R I: Immunologic monitoring. Immunol Rev 196:247-264, 2003
51. Gebel H M, Bray R A, Nickerson P: Pre-transplant assessment of donor-reactive, HLA-specific antibodies in renal transplantation: contraindication vs. risk. Am J Transplant 3:1488-1500, 2003
52. Heeger P S, Greenspan N S, Kuhlenschmidt S, Dejelo C, Hricik D E, Schulak J A, Tary-Lehmann M: Pretransplant frequency of donor-specific, IFN-gamma-producing lymphocytes is a manifestation of immunologic memory and correlates with the risk of posttransplant rejection episodes. J Immunol 163:2267-2275, 1999
53. Hricik D E, Rodriguez V, Riley J, Bryan K, Tary-Lehmann M, Greenspan N, Dejelo C, Schulak J A, Heeger P S: Enzyme linked immunosorbent spot (ELISPOT) assay for interferon-gamma independently predicts renal function in kidney transplant recipients. Am J Transplant 3:878-884, 2003
54. Roberti I, Panico M, Reisman L: Urine flow cytometry as a tool to differentiate acute allograft rejection from other causes of acute renal graft dysfunction. Transplantation 64:731-734, 1997
55. Roberti I and Reisman L: Serial evaluation of cell surface markers for immune activation after acute renal allograft rejection by urine flow cytometry-correlation with clinical outcome. Transplantation 71:1317-1320, 2001
56. Ding R, Li B, Muthukumar T, Dadhania D, Medeiros M, Hartono C, Serur D, Seshan S V, Sharma V K, Kapur S, Suthanthiran M: CD103 mRNA levels in urinary cells predict acute rejection of renal allografts. Transplantation 15, 75:1307-312, 2003
57. Li B, Hartono C, Ding R, Sharma V K, Ramaswamy R, Qian B, Serur D, Mouradian J, Schwartz J E, Suthanthiran M: Noninvasive diagnosis of renal-allograft rejection by measurement of messenger RNA for perforin and granzyme B in urine. N Engl J Med 344:947-954, 2001
58. Simon T, Opelz G, Wiesel M, Ott R C, Susal C: Serial peripheral blood perforin and granzyme B gene expression measurements for prediction of acute rejection in kidney graft recipients. Am J Transplant 3:1121-1127, 2003
59. Sadeghi M, Daniel V, Wiesel M, Hergesell O, Opelz G: High urine sIL-6R as a predictor of late graft failure in renal transplant recipients. Transplantation 76:1190-1194, 2003
60. Hu H, Aizenstein B D, Puchalski A, Burmania J A, Hamawy M M, Knechtle S J: Elevation of CXCR3-binding chemokines in urine indicates acute renal-allograft dysfunction, Am J Transplant 4:432-437, 2004
61. Karpinski M, Rush D, Jeffery J, Pochinco D, Milley D, Nickerson P: Heightened Peripheral Blood Lymphocyte CD69 Expression is Neither Sensitive nor Specific as a Noninvasive Diagnostic Test for Renal Allograft Rejection. J Am Soc Nephrol 14:226-233, 2003
62. McLean A: What goes around (in kidney transplant rejection) does not necessarily come around (in the blood). Am J Transplant 3:1045-1046, 2003
63. Lachenbruch P A, Rosenberg A S, Bonvini E, Cavaille-Coll M W, Colvin R B: Biomarkers and Surrogate Endpoints in Renal Transplantation: Present Status and Considerations for Clinical Trial Design. American Journal of Transplantation 4:451-457, 2004
64. Lang T and Secic M: How to report statistics in medicine. ACP-Medical writing and communications. 147-169, 1997
65. van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van d, V, Bartelink H, Rodenhuis S, Rutgers E T, Friend S H, Bernards R: A gene-expression signature as a predictor of survival in breast cancer. N Engl J Med 347:1999-2009, 2002
66. Carrette O, Demalte I, Scherl A, Yalkinoglu O, Corthals G, Burkhard P, Hochstrasser D F, Sanchez J C: A panel of cerebrospinal fluid potential biomarkers for the diagnosis of Alzheimer's disease. Proteomics 3:1486-1494, 2003
67. Guillaume E, Zimmermann C, Burkhard P R, Hochstrasser D F, Sanchez J C: A potential cerebrospinal fluid and plasmatic marker for the diagnosis of Creutzfeldt-Jakob disease. Proteomics 3:1495-1499, 2003
68. Knepper M A: Proteomics and the kidney. J Am Soc Nephrol 13:1398-1408, 2002
69. Hanash S: Disease proteomics. Nature 422:226-232, 2003
70. Gygi S P, Rist B, Gerber S A, Turecek F, Gelb M H, Aebersold R: Quantitative analysis of complex protein mixtures using isotope-coded affinity tags. Nat Biotechnol 17:994-999, 1999
71. Zhou H, Ranish J A, Watts J D, Aebersold R: Quantitative proteome analysis by solid-phase isotope tagging and mass spectrometry. Nat Biotechnol 20:512-515, 2002
72. Shevchenko A, Chemushevich I, Ens W, Standing K G, Thomson B, Wilm M, Mann M: Rapid ‘de novo’ peptide sequencing by a combination of nanoelectrospray, isotopic labeling and a quadrupole/time-of-flight mass spectrometer. Rapid Commun Mass Spectrom 11:1015-1024, 1997
73. Stewart I I, Thomson T, Figeys D: 18O labeling: a tool for proteomics. Rapid Commun Mass Spectrom 15:2456-2465, 2001
74. Yao X, Afonso C, Fenselau C: Dissection of proteolytic 18O labeling: endoprotease-catalyzed 16O-to-18O exchange of truncated peptide substrates. J Proteome Res 2:147-152, 2003
75. D'Amico G and Bazzi C: Pathophysiology of proteinuria. Kidney Int 63:809-825, 2003
76. Russo L M, Bakris G L, Comper W D: Renal handling of albumin: a critical review of basic concepts and perspective. Am J Kidney Dis 39:899-919, 2002
77. Marino M, Andrews D, Brown D, McCluskey R T: Transcytosis of retinol-binding protein across renal proximal tubule cells after megalin (gp 330)-mediated endocytosis. J Am Soc Nephrol 12:637-648, 2001
78. Gudehithlu K P, Pegoraro A A, Dunea G, Arruda J A, Singh A K: Degradation of albumin by the renal proximal tubule cells and the subsequent fate of its fragments. Kidney Int 65:2113-2122, 2004
79. Kokot F and Dulawa J: Tamm-Horsfall protein updated. Nephron 85:97-102, 2000
80. Prescott L F: The normal urinary excretion rates of renal tubular cells, leucocytes and red blood cells. Clin Sci 31:425-435, 1966
81. Loboda A V, Krutchinsky A N, Bromirski M, Ens W, Standing K G: A tandem quadrupole/time of-flight mass spectrometer with a matrix-assisted laser desorption/ionization source: design and performance. Rapid Commun Mass Spectrom 14:1047-1057, 2000
82. Hillenkamp F, Karas M, Beavis R C, Chait B T: Matrix-assisted laser desorption/ionization mass spectrometry of biopolymers. Anal Chem 63:1193 A-1203A, 1991
83. Tencer J, Thysell H, Andersson K, Grubb A: Stability of albumin, protein H C, immunoglobulin G, kappa- and lambda-chain immunoreactivity, orosomucoid and alpha 1-antitrypsin in urine stored at various conditions. Scand J Clin Lab Invest 54:199-206, 1994
84. Froom P, Bieganiec B, Ehrenrich Z, Barak M: Stability of common analytes in urine refrigerated for 24 h before automated analysis by test strips. Clin Chem 46:1384-1386, 2000
85. Innanen V T, Groom B M, de Campos F M: Microalbumin and freezing. Clin Chem 43:1093-1094, 1997
86. Schultz C J, Dalton R N, Turner C, Neil H A, Dunger D B: Freezing method affects the concentration and variability of urine proteins and the interpretation of data on microalbuminuria. The Oxford Regional Prospective Study Group. Diabet Med 17:7-14, 2000
87. Ganz T: Defensins in the urinary tract and other tissues. J Infect Dis 183 Suppl 1:S41-S42, 2001
88. Kunin C M, Evans C, Bartholomew D, Bates D G: The antimicrobial defense mechanism of the female urethra: a reassessment. J Urol 168:413-419, 2002
89. Hampel D J, Sansome C, Sha M, Brodsky S, Lawson W E, Goligorsky M S: Toward proteomics in uroscopy: urinary protein profiles after radiocontrast medium administration. J Am Soc Nephrol 12:1026-1035, 2001
90. Zhang L, Yu W, He T, Yu J, Caffrey R E, Dalmasso E A, Fu S, Pham T, Mei J, Ho J J, Zhang W, Lopez P., Ho D D: Contribution of human alpha-defensin 1, 2, and 3 to the anti-HIV-1 activity of CD8 antiviral factor. Science 298:995-1000, 2002
91. Cazares L H, Adam B L, Ward M D, Nasim S, Schellhammer P F, Semmes O J, Wright G L, Jr.: Normal, benign, preneoplastic, and malignant prostate cells have distinct protein expression profiles resolved by surface enhanced laser desorption/ionization mass spectrometry. Clin Cancer Res 8:2541-2552, 2002
92. Wright G L, Jr.: SELDI proteinchip MS: a platform for biomarker discovery and cancer diagnosis. Expert Rev Mol Diagn 2:549-563, 2002
93. Yip T T and Lomas L: SELDI ProteinChip array in oncoproteomic research. Technol Cancer Res Treat 1:273-280, 2002
94. Tirumalai R S, Chan K C, Prieto D A, Issaq H J, Conrads T P, Veenstra T D: Characterization of the low molecular weight human serum proteome. Mol Cell Proteomics 2:1096-1103, 2003
95. Diamandis E P: Mass spectrometry as a diagnostic and a cancer biomarker discovery tool: opportunities and potential limitations. Mol Cell Proteomics 3:367-378, 2004
96. Schaub S, Wilkins J, Weiler T, Sangster K, Rush D, Nickerson P: Urine protein profiling with surface-enhanced laser-desorption/ionization time-of-flight mass spectrometry. Kidney Int 65:323-332, 2004
97. Najafian N, Salama A D, Fedoseyeva E V, Benichou G, Sayegh M H: Enzyme-linked immunosorbent spot assay analysis of peripheral blood lymphocyte reactivity to donor HLADR peptides: potential novel assay for prediction of outcomes for renal transplant recipients. J Am Soc Nephrol 13:252-259, 2002
98. Clarke W, Silverman B C, Zhang Z, Chan D W, Klein A S, Molmenti E P: Characterization of renal allograft rejection by urinary proteomic analysis. Ann Surg 237:660-665, 2003
99. Petricoin E F, Ardekani A M, Hitt B A, Levine P J, Fusaro V A, Steinberg S M, Mills G B, Simone C, Fishman D A, Kohn E C, Liotta L A: Use of proteomic patterns in serum to identify ovarian cancer. Lancet 359:572-577, 2002
100. Sorof J M, Vartanian R K, Olson J L, Tomlanovich S J, Vincenti F G, Amend W J: Histopathological concordance of paired renal allograft biopsy cores. Effect on the diagnosis and management of acute rejection. Transplantation 60:1215-1219, 1995
101. Sagedal S, Nordal K P, Hartmann A, Degre M, Holter E, Foss A, Osnes K, Leivestad T, Fauchald P, Rollag H: A prospective study of the natural course of cytomegalovirus infection and disease in renal allograft recipients. Transplantation 70:1166-1174, 2000
102. Abecassis M M, Koffron A J, Kaplan B, Buckingham M, Muldoon J P, Cribbins A J, Kaufman D B, Fryer J P, Stuart J, Stuart F P: The role of PCR in the diagnosis and management of CMV in solid organ recipients: what is the predictive value for the development of disease and should PCR be used to guide antiviral therapy? Transplantation 63:275-279, 1997
103. Andersen C B, Ladefoged S D, Lauritsen H K, Hansen P R, Larsen S: Detection of CMV DNA and CMV antigen in renal allograft biopsies by in situ hybridisation and immunohistochemistry. Nephrol Dial Transplant 5:1045-1050, 1990
104. Kashyap R, Shapiro R, Jordan M, Randhawa P S: The clinical significance of cytomegaloviral inclusions in the allograft kidney. Transplantation 67:98-103, 1999
105. Jones M D, Patterson S D, Lu H S: Determination of disulfide bonds in highly bridged disulfide linked peptides by matrix-assisted laser desorption/ionization mass spectrometry with postsource decay. Anal Chem 70:136-143, 1998
106. Patterson S D and Katta V: Prompt fragmentation of disulfide-linked peptides during matrix assisted laser desorption ionization mass spectrometry. Anal Chem 66:3727-3732, 1994
107. Kudo S, Miyamoto G, Kawano K: Proteases involved in the metabolic degradation of human interleukin-1beta by rat kidney lysosomes. J Interferon Cytokine Res 19:361-367, 1999
108. Mori K, Shimizu H, Konno A, Iwanaga T: Immunohistochemical localization of napsin and its potential role in protein catabolism in renal proximal tubules. Arch Histol Cytol 65:359-368, 2002
109. Goto M, Mizunashi K, Kimura N, Furukawa Y: Decreased sensitivity of distal nephron and collecting duct to parathyroid hormone in pseudohypoparathyroidism type I. J Am Soc Nephrol
   12:1965-1970, 2001
110. Krokhin O, Li Y, Andonov A, Feldmann, H, Flick R, Jones S, Stroeher U, Bastien N, Dasuri K V, Cheng K, Simonsen J N, Perreault H, Wilkins J, Ens W, Plummer F, Standing K G: Mass Spectrometric Characterization of Proteins from the SARS Virus: A Preliminary Report. Mol Cell Proteomics 2:346-356, 2003
111. Schardijn G, Statius van Eps L W, Swaak A J, Kager J C, Persijn J P: Urinary beta 2 microglobulin in upper and lower urinary-tract infections. Lancet 1:805-807, 1979
112. Davey P G and Gosling P: beta 2-Microglobulin instability in pathological urine. Clin Chem 28:1330-1333, 1982
113. Yamamoto H, Yamada T, Itoh Y: Probable involvement of cathepsin D in the degradation of beta2-microglobulin in acidic urine. Clin Chem Lab Med 38:495-499, 2000
114. Schauer-Vukasinovic V, Langen H, Giller T: Detection of immunoreactive napsin A in human urine. Biochim Biophys Acta 1524:51-56, 2001
115. Schauer-Vukasinovic V, Bur D, Kling D, Gruninger F, Giller T: Human napsin A: expression, immunochemical detection, and tissue localization. FEBS Lett 462:135-139, 1999
116. Rush D, Nickerson P, Gough J, McKenna R, Grimm P, Cheang M, Trpkov K, Solez K, Jeffery J: Beneficial effects of treatment of early subclinical rejection: a randomized study. J Am Soc Nephrol 9:2129-2134, 1998
117. Nankivell B J, Borrows R J, Fung C L, O'Connell P J, Allen R D, Chapman J R: Natural history, risk factors, and impact of subclinical rejection in kidney transplantation. Transplantation 78:242-249, 2004
118. Hewitt S M, Dear J, Star R A: Discovery of protein biomarkers for renal diseases. J Am Soc Nephrol 15:1677-1689, 2004
119. D'Amico G and Bazzi C: Urinary protein and enzyme excretion as markers of tubular damage. Curr. Opin. Nephrol. and Hypertension 12:639-643, 2003

TABLE 1

| | Gene-microarrays | MS-based proteomics |
|---|---|---|
| Advantages | Capturing of known mRNA molecules | Proteins are the final effector molecules |
| | (identification is not a problem) | Proteins are the main target for therapeutics |
| | DNA and RNA are conservative | Applicable to biological fluids, e.g. ascites |
| | High sensitivity even in complex mixtures | and urine |
| | Amplification possible | |
| Disadvantages | Provides no information regarding protein | No amplification |
| | expression levels | Not quantitative |
| | Reproducibility, especially for genes with | Comparison and identification is |
| | low expression levels | complicated by high dynamic range of |
| | Dependant on cells (difficult to apply to | proteins (mmol to fmol) and ion suppression |
| | biological fluids with low cells counts, e.g. | Reproducibility of high-throughput |
| | ascites and urine) | technologies (SELDI-TOF-MS, ICAT) |
| | Larger sample amounts needed for analysis | Proteins are not conservative (e.g. |
| | | phosphorylation, glycosylation) |

Most human tissues are a mixture of different cells types. Changes in gene and protein expression patterns may just be related to different cell type composition of the tissue sample, rather then a ‘real’ up- or down-regulation. This bias can be overcome with cell type sampling by laser-capture-microdissection

TABLE 2

| | | | SELDI- | |
|---|---|---|---|---|
| | 2-DE | LC-MS | TOF-MS | ELISA |
| Use for biomarker | Yes | Yes | Yes | Limited* |
| discovery | | | | |
| Direct identification | Yes | Yes | No | No |
| of detected biomarkers | | | | |
| Sensitivity | Medium | High | Medium | Highest |
| Throughput | Low | Low | Highest | High |

TABLE 3
Acute Banff Score
i0 - No or trivial interstitial inflammation (<10% of unscarred
parenchyma)
i1 - 10 to 25% of parenchyma inflamed
i2 - 26 to 50% of parenchyma inflamed
i3 - more than 50% of parenchyma inflamed
Indicate the presence of remarkable numbers of eosinophils, PMNL,
or plasma cells (specify which) with an asterisk (*).
t0 - No mononuclear cells in tubules
t1 - Foci with 1 to 4 cells/tubular cross section (or 10 tubular cells)
t2 - Foci with 5 to 10 cells/tubular cross section
t3 - Foci with >10 cells/tubular cross section, or the presence of at
least two areas of tubular basement membrane destruction accompa-
nied by i2/i3 inflammation and t2 tubulitis elsewhere in the biopsy
^{a}Applies to tubules no more than mildly atrophic
g0 - No glomerulitis
g1 - Glomerulitis in less than 25% of glomeruli
g2 - Segmental or global glomerulitis in 25 to 75% of glomeruli
g3 - Glomerulitis (mostly global) in more than 75% of glomeruli
v0 - No arteritis
v1 - Mild-to-moderate intimal arteritis in at least one arterial cross
section
v2 - Severe intimal arteritis with at least 25% luminal area lost in at
least one arterial cross section
v3 - Transmural arteritis and/or arterial fibrinoid change and medial
smooth muscle necrosis with lymphocytic infiltrate in vessel
Note number of arteries present and number affected. Indicate
infarction and/or interstitial hemorrhage by an asterisk (with any
level v score).
Chronic Banff Score
ci0 - Interstitial fibrosis in up to 5% of cortical area
ci1 - Mild - interstitial fibrosis in 6 to 25% of cortical area
ci2 - Moderate - interstitial fibrosis in 26 to 50% of cortical area
ci3 - Severe - interstitial fibrosis in >50% of cortical area
ct0 - No tubular atrophy
ct1 - Tubular atrophy in up to 25% of the area of cortical tubules
ct2 - Tubular atrophy involving 26 to 50% of the area of cortical
tubules
ct3 - Tubular atrophy in >50% of the area of cortical tubules
cg0 - No glomerulopathy - double contours in <10% of peripheral
capillary loops in most severely affected glomerulus
cg1 - Double contours affecting up to 25% of peripheral capillary loops
in the most affected of nonsclerotic glomeruli
cg2 - Double contours affecting 26 to 50% of peripheral capillary loops
in the most affected of nonsclerotic glomeruli
cg3 - Double contours affecting more than 50% of peripheral capillary
loops in the most affected of nonsclerotic glomeruli
Note the number of glomeruli and percentage sclerotic.
cv0 - No chronic vascular changes
cv1 - Vascular narrowing of up to 25% luminal area by fibrointimal
thickening of arteries ± breach of internal elastic lamina or presence
of foam cells or occasional mononuclear cells^{a}
cv2 - Increased severity of changes described above with 26 to 50%
narrowing of vascular luminal area^{a}
cv3 - Severe vascular changes with >50% narrowing of vascular luminal
area^{a}
^{a}In the most severely affected vessel. Note if lesions characteristic
of chronic rejection (breaks in the elastica, inflammatory cells in
fibrosis, formation of neointima) are seen.

TABLE 4

| | | | Acute | | | Recurrent or |
|---|---|---|---|---|---|---|
| | Stable | | clinical | | | de novo |
| | transplant | | rejection | | ATN | glomerulopathy |
| Variable | (n = 22) | | (n = 18) | | (n = 5) | (n = 5) |
| Female Sex - no. (%) | 12 | (55) | 6 | (33) | 2 | 2 |
| Age - mean ± SD | 45 ± 13 | | 43 ± 10 | | 40 ± 18 | 47 ± 9 |
| Caucasian Race - no. (%) | 14 | (64) | 15 | (83) | 3 | 5 |

Nephropathy

| | | | | | | |
|---|---|---|---|---|---|---|
| Diabetic - no. (%) | 6 | (27) | 3 | (17) | 1 | 0 |
| Glomerulonephritis - no. (%) | 6 | (27) | 6 | (33) | 3 | 4 |
| others - no. (%) | 10 | (46) | 9 | (50) | 1 | 1 |
| First transplant - no. (%) | 21 | (95) | 16 | (89) | 5 | 4 |
| Cadaveric donor - no. (%) | 15 | (68) | 10 | (56) | 3 | 5 |
| HLA-mismatches - median (range) | 3 | (1-5) | 4 | (2-5)^{a)} | 3 (2-4) | 3 (3-5) |

Panel-reactive antibodies (PRA)

| Peak PRA > 10% - no. (%) | 2 | (9) | 0 | | 0 | 1 |
|---|---|---|---|---|---|---|
| Current PRA > 10% - no. (%) | 1 | (5) | 0 | | 0 | 1 |
| Cytomegalovirus serology | 3 | (14) | 3 | (17) | 1 | 1 |
| Recipient neg./Donor pos. - no. (%) | 7 | (32) | 4 | (22) | 0 | 2 |
| Recipient neg./Donor neg. - no. (%) | 4 | (18) | 9 | (50) | 3 | 1 |
| Recipient pos./Donor pos. - no. (%) | 8 | (36) | 2 | (11) | 1 | 1 |
| Recipient pos./Donor neg. - no. (%) | | | | | | |

Allograft biopsy

| | | | | | | |
|---|---|---|---|---|---|---|
| Week post transplant - median (range) | 8 | (3-51) | 8 | (1-18) | day 5 or 6^{d)} | 253 (7-442) |

Rejection type (Banff 1997)

| IA (moderate tubulitis) - no. (%) | | | 7 | (39) | | |
|---|---|---|---|---|---|---|
| IB (severe tubulitis) - no. (%) | | | 8 | (44) | | |
| IIA (moderate arteritis) - no. (%) | | | 3 | (17) | | |
| Creatinine at biopsy [μmol/L] - mean ± SD | 91 ± 26 | | 180 ± 59^{b)} | | 942 ± 80^{e)} | 122 ± 29 |
| % above baseline - median (range) | | | 25 | (11-76) | | |
| Proteinuria at biopsy [g/L] - median (range) | 0.07^{c)} | (0.03-0.17) | 0.09 | (0.03-0.28) | | 3.20 (0.58-6.00) |

^{a)}P = 0.003 vs. stable transplant group
^{b)}P < 0.001 vs. stable transplant group
^{c)}P = 0.14 vs. acute clinical rejection group. P < 0.001 vs, recurrent or de novo glomerulopathy group
^{d)}Not included for statistical analysis
^{e)}Not included for statistical comparison (3 of 5 patients were on hemodialysis)

TABLE 5

| | Normal | Rejection | |
|---|---|---|---|
| | pattern | pattern | |
| CMV-viremia | (n = 19)^{a)} | (n = 21)^{b)} | P-value |
| CMV-DNA positive - no. | 3 | 2 | |
| CMV-DNA negative - no. | 10 | 12 | P = 0.83 |
| No CMV-PCR available - no. | 6 ^{c)} | 7 ^{d)} | |

^{a)} Consists of 18 patients from the stable transplant group plus 1 patient from the acute clinical rejection group
^{b)} Consists of 4 patients from the stable transplant group plus 17 patients from the acute clinical rejection group
^{c)} CMV-PCR was not performed for the following reasons: CMV sero-negativity of both donor and recipient (n = 2); test was not ordered (n = 3); or only CMV pp65-antigen was evaluated (n = 1; patient tested negative)
^{d)} CMV-PCR was not performed for the following reasons: CMV sero-negativity of both donor and recipient (n = 3); test was not ordered (n = 3); or only CMV pp65-antigen was evaluated (n = 1; patient tested negative)

TABLE 6

| | | Mass | Mass found | Delta | Sequence | | |
|---|---|---|---|---|---|---|---|
| Amino | | calculated | by LC-MS | Mass | confirmed | | |
| acid # | Peptide fragments | (Da) | (Da) | (mDa) | by MS/MS | Comment | SEQ ID NO: |
| 1-3 | IQR | 416.262 | 416.266 | 4 | + | N-terminal peptide | 11 |
| 4-6 | TPK | 345.214 | 345.213 | 1 | + | | 12 |
| 7-12 | IQVYSR | 765.426 | 765.430 | 4 | + | | 13 |
| 13-19 | HPAENGK | 752.369 | 753.354 | 1 | + | N17 deamidation | 14 |
| 20-41 | SNFLNCYVSGFHPSDIEVDLLK | 2554.229 | 2554.240 | 11 | + | | 15 |
| 21-41 | NFLNCYVSGFHPSDIEVDLLK | 2467.197 | 2467.207 | 10 | + | non-tryptic | 16 |
| 22-41 | FLNCYVSGFHPSDIEVDLLK | 2353.154 | 2353.169 | 15 | + | non-tryptic | 17 |
| 23-41 | LNCYVSGFHPSDIEVDLLK | 2206.085 | 2206.112 | 27 | + | non-tryptic | 18 |
| 25-41 | CYVSGFHPSDIEVDLLK | 1978.958 | 1978.972 | 14 | + | non-tryptic | 19 |
| 26-41 | YVSGFHPSDIEVDLLK | 1818.928 | 1818.943 | 15 | + | non-tryptic | 20 |
| 27-41 | VSGFHPSDIEVDLLK | 1655.865 | 1655.870 | 5 | + | non-tryptic | 21 |
| 28-41 | SGFHPSDIEVDLLK | 1556.796 | 1556.800 | 4 | + | non-tryptic | 22 |
| 29-41 | GFHPSDIEVDLLK | 1469.764 | 1469.764 | 0 | + | non-tryptic | 23 |
| 31-41 | HPSDIEVDLLK | 1265.674 | 1265.671 | 3 | + | non-tryptic | 24 |
| 20-40 | SNFLNCYVSGFHPSDIEVDLL | 2426.134 | 2426.125 | 9 | + | non-tryptic | 25 |
| 20-39 | SNFLNCYVSGFHPSDIEVDL | 2313.050 | 2313.043 | 7 | + | non-tryptic | 26 |
| 20-38 | SNFLNCYVSGFHPSDIEVD | 2199.966 | 2199.994 | 28 | + | non-tryptic | 27 |
| 42-45 | NGER | 475.226 | 476.217 | 7 | + | N42 deamidation | 28 |
| 46-48 | IEK | 389.240 | 389.247 | 7 | + | | 29 |
| 49-58 | VEHSDLSFSK | 1148.559 | 1148.566 | 7 | + | | 30 |
| 46-56 | IEKVEHSDLSF | 1303.654 | 1303.652 | 1 | + | non-tryptic | 31 |
| 46-57 | IEKVEHSDLSFS | 1390.686 | 1390.695 | 9 | + | non-tryptic | 32 |
| 46-59 | IEKVEHSDLSFSKD | 1633.808 | 1633.821 | 14 | + | non-tryptic | 33 |
| 46-60 | IEKVEHSDLSFSKDW | 1819.887 | 1819.886 | 1 | + | non-tryptic | 34 |
| 46-61 | IEKVEHSDLSFSKDWS | 1906.919 | 1906.927 | 8 | + | non-tryptic | 35 |
| 46-62 | IEKVEHSDLSFSKDWSF | 2053.987 | 2054.004 | 17 | + | non-tryptic | 36 |
| 46-63 | IEKVEHSDLSFSKDWSFY | 2217.056 | 2217.064 | 8 | + | non-tryptic | 37 |
| 76-81 | DEYACR | 813.320 | 813.324 | 4 | + | | 38 |
| 69-81 | EFTPTEKDEYACR | 1645.717 | 1645.720 | 3 | + | non-tryptic | 39 |
| 70-81 | FTPTEKDEYACR | 1516.674 | 1516.682 | 8 | + | non-tryptic | 40 |
| 71-81 | TPTEKDEYACR | 1369.606 | 1369.614 | 8 | + | non-tryptic | 41 |
| 72-81 | PTEKDEYACR | 1268.558 | 1268.576 | 18 | + | non-tryptic | 42 |
| 73-81 | TEKDEYACR | 1171.505 | 1171.518 | 13 | + | non-tryptic | 43 |
| 74-81 | EKDEYACR | 1070.458 | 1070.461 | 3 | + | non-tryptic | 44 |
| 75-81 | KDEYACR | 941.415 | 941.424 | 9 | + | non-tryptic | 45 |
| 82-91 | VNHVTLSQPK | 1122.627 | 1122.631 | 4 | + | | 46 |
| 92-94 | IVK | 359.266 | 359.270 | 4 | + | | 47 |
| 95-99 | WDRDM | 722.293 | 722.297 | 4 | + | C-terminal peptide | 48 |
| 95-97 | WDR | 476.226 | 476.226 | 0 | + | | 49 |

1. A method of detecting kidney dysfunction in an animal comprising:
(a) testing a sample from the animal for the presence of β2-microglobulin protein fragments, wherein the presence of one or more β2-microglobulin protein fragments when compared to a control sample indicates that the animal has kidney dysfunction.

2. A method according to claim 1 wherein the β2-microglobulin protein fragments are one or more than one of the fragments selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

3. A method according to claim 1 wherein the presence of β2-microglobulin protein fragments is determined using at least one antibody.

4. A method according to claim 1 wherein the presence of β2-microglobulin protein fragments is determined using at least one aptamer.

5. A method according to claim 1 wherein the presence of β2-microglobulin protein fragments is determined using mass spectrometry.

6. A method according to claim 5 wherein the mass spectrometry is surface-enhanced laser desorption/ionization time-of-flight (SELDI-TOF) mass spectrometry.

7. A method according to claim 1 wherein the sample is urine.

8. A method of monitoring kidney function in an animal comprising:
(a) testing a sample from the animal to determine the level of β2-microglobulin protein fragments;
(b) repeating step (a) at a later point in time and comparing the result obtained in step (a) with the result obtained in step (b) wherein a difference in the level of β2-microglobulin protein fragments is indicative of a change in kidney function.

9. A method according to claim 8 wherein the β2-microglobulin protein fragments are one or more than one of the fragments selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

10. A method according to claim 8 wherein the level of β2-microglobulin protein fragments is determined using at least one antibody.

11. A method according to claim 8 wherein the level of β2-microglobulin protein fragments is determined using at least one aptamer.

12. A method according to claim 8 wherein the level of β2-microglobulin protein fragments is determined using mass spectrometry.

13. A method according to claim 12 wherein the mass spectrometry is surface-enhanced laser desorption/ionization time-of-flight (SELDI-TOF) mass spectrometry.

14. A method according to claim 8 wherein the sample is urine.

15. A method of detecting kidney transplant related disease in an animal that has received a transplant comprising:
(a) testing a sample from the animal for the presence of β2-microglobulin protein fragments, wherein the presence of one or more β2-microglobulin protein fragments when compared to a sample from a normal animal indicates that the animal has a kidney transplant related disease.

16. A method according to claim 15 wherein the β2-microglobulin protein fragments are one or more than one of the fragments selected from the group consisting of I1-Y63 (SEQ ID NO:2), I1-F62 (SEQ ID NO:3), I1-S61 (SEQ ID NO:4), E69-M99 (SEQ ID NO:5), F70-M99 (SEQ ID NO:6), Y66-M99 (SEQ ID NO:7), Y67-M99 (SEQ ID NO:8) and T68-M99 (SEQ ID NO:9).

17. A method according claim 15 wherein the presence of β2-microglobulin protein fragments is determined using at least one antibody.

18. A method according to claim 15 wherein the presence of β2-microglobulin protein fragments is determined using at least one aptamer.

19. A method according to claim 15 wherein the presence of β2-microglobulin protein fragments is determined using mass spectrometry.

20. A method according to claim 19 wherein the mass spectrometry is surface-enhanced laser desorption/ionization time-of-flight (SELDI-TOF) mass spectrometry.

21. A method according to claim 15 wherein the kidney transplant related disease is transplant rejection.

22. A method according to claim 15 wherein the sample is urine.

23. A method of detecting kidney dysfunction in an animal comprising:
(a) testing a urine sample from the animal for protease activity, wherein increased protease activity when compared to a control sample indicates that the animal has kidney dysfunction.

24. A method according to claim 23 wherein the urine sample from the animal is tested for aspartic protease activity.

25. A method according to claim 24 wherein the urine sample from the animal is tested for the activity of an aspartic protease selected from the group consisting of cathepsin D and napsin A.

26. A kit for detecting kidney dysfunction in an animal comprising (i) reagents for conducting a method according to claim 1 and (ii) instructions for its use.

27. A kit according to claim 26 wherein the reagents comprise antibodies specific to at least one β2-microglobulin protein fragment.

28. A kit according to claim 27 wherein the reagents comprise antibodies that recognize both intact β2-microglobulin and a β2-microglobulin protein fragment, and antibodies specific for intact β2-microglobulin.

29. A kit for monitoring kidney function in an animal comprising (i) reagents for conducting a method according to claim 8 and (ii) instructions for its use.

30. A kit according to claim 29 wherein the reagents comprise antibodies specific for at least one β2-microglobulin protein fragment.

31. A kit for detecting kidney transplant related disease in an animal who has received a transplant comprising (i) reagents for conducting a method according to claim 15 and (ii) instructions for its use.

32. A kit according to claim 31 wherein the reagents comprise antibodies specific for at least one β2-microglobulin protein fragment.

33. A kit according to claim 32 wherein the reagents comprise antibodies that recognize both intact β2-microglobulin and a β2-microglobulin protein fragment, and antibodies specific for intact β2-microglobulin.

34. A kit for detecting kidney dysfunction in an animal comprising (i) reagents for conducting a method according to claim 23 and (ii) instructions for its use.
